(19) United States
(12) Patent Application Publication
(10) Pub. No.: US 2021/0318318 A1
(43) Pub. Date: Oct. 14, 2021

(54) Kinase Activity In Tumors

(71) Applicant: Institute For Cancer Research d/b/a The Research Institute of Fox Chase Cancer Center, Philadelphia, PA (US)

(72) Inventor: James S. Duncan, Philadelphia, PA (US)

(21) Appl. No.: 17/271,712

(22) PCT Filed: Aug. 25, 2019
(86) PCT No.: PCT/US2019/048053

§ 371 Date: Feb. 26, 2021

Related U.S. Application Data
(60) Provisional application No. 62/725,369, filed on Aug. 31, 2018.

(51) Int. Cl. G01N 33/574; C12N 9/12

(52) U.S. Cl. CPC G01N 33/57449; G01N 2333/912; C12N 9/12

(57) ABSTRACT

The present disclosure provides a multi-analyte column comprising at least four layers least four layers of multiplexer inhibitor beads, wherein each layer has specific binding affinity for multiple kinases, and methods of generating a kinome profile of a cell, of diagnosing cancer based on the kinome profile of a patient, of determining a cancer treatment regimen, assessing a treatment regimen, or improving the effectiveness of a treatment regimen based on the kinome profile of a patient, and methods for predicting the development of resistance to a chemotherapy regimen based on the kinome profile of a patient.

### FIELD

The present disclosure is directed, in part, to multi-analyte columns comprising at least four layers of multiplexer inhibitor beads, wherein each layer has specific binding affinity for multiple kinases, and to methods of generating a kinome profile of a cell, of diagnosing cancer based on the kinome profile of a patient, of determining a cancer treatment regimen, assessing a treatment regimen, or improving the effectiveness of a treatment regimen based on the kinome profile of a patient, and for predicting the development of resistance to a chemotherapy regimen based on the kinome profile of a patient.

### BACKGROUND

Various references, including patents, patent applications, accession numbers, technical articles, and scholarly articles are cited throughout the specification. Each reference is incorporated by reference herein, in its entirety and for all purposes.

Protein kinases are a family of about 535 enzymes that, collectively, are termed the kinome (Manning et al., Science, 2002, 298, 1912-1934). Uncontrolled protein kinase activity has been linked to the development of nearly 25% of all cancers; consequently, protein kinases represent one of the most promising avenues for cancer therapy (Knight et al., Nat. Rev. Cancer, 2010, 10, 130-137; and Metz et al., Nat. Chem. Biol., 2011, 7, 200-202). Indeed, more than 30 kinase-specific inhibitors are currently approved for therapy of various cancer types, with more than 150 kinase inhibitors in Phase 1-3 clinical trials across all diseases (Klaeger et al., Science, 2017, 358, eaan4368). However, most of these kinase-specific inhibitors target a relatively small fraction of the human kinome with only about 20% avidly being explored as primary targets for drug therapy (Fedorov et al., Nat. Rev. Cancer, 2016, 16, 83-98; and Fleuren et al., Nat. Rev. Cancer, 2016, 16, 83-98). Thus, the majority of the kinome remains untargeted for cancer therapy and about 50% of the kinome is largely uncharacterized with respect to the function and role of these kinases in cancer, representing the ‘dark’ cancer (Drewry et al., PLoS One, 2017, 12, e0181585; Fedorov et al., Nat. Rev. Cancer, 2016, 16, 83-98; and Knapp et al., Nat. Chem. Biol., 2013, 9, 3-6). Notably, several CRISPR/cas9 and/or RNAi loss-of-function studies have shown that many dark kinases are essential for cancer cell viability highlighting the therapeutic potential of the dark kinome for the treatment of cancer (Barbie et al., Nature, 2009, 462, 108-112; and Scholl et al., Cell, 2009, 137, 821-834).

However molecularly targeted cancer therapies can fail when tumor cells circumvent the action of a single inhibitor, facilitating the development of resistance. Acquired or selected mutations can decrease affinity for therapeutic kinase inhibitors, but resistance also develops by alternate kinase activation bypassing the action of a highly specific inhibitor (Chandarlapaty et al., Cancer Cell, 2011, 19, 58-71; Hochgrafe et al., Cancer Research, 2010, 70, 9391-9401; Johannessen et al., Nature, 2010, 468, 968-972; Nazarian et al., Nature, 2010, 468, 973-977; Sun et al., Cancer Cell, 2011, 18, 683-695; and Villanueva et al., Cancer Cell, 2010, 18, 683-695).

High-grade serous ovarian carcinoma (HGSOC) is one of the most common and lethal forms of ovarian carcinoma, and current treatments have only modestly impacted survival. These tumors are characterized by near-universal loss of the TP53 tumor suppressor gene, and consequently, exhibit genome instability and aberrant signaling. Signaling abnormalities in HGSOCs, particularly those involving over-activated protein kinases, represent potential therapeutic avenues (Coscia et al., Nat. Commun., 2016, 7, 12645; Verhaak et al., J. Clin. Invest., 2013, 123, 517-525; and Zhang et al., Cell, 2016, 166, 755-765). However, single agent therapies targeting overactive EGFR, HER2, IGF1R, SRC, AKT, MEK or Wee1 have shown limited therapeutic benefit in HGSOC due to drug resistance (Garcia et al., Gynecol. Oncol, 2012, 124, 569-574; Pinato et al., Cancer Treat. Rev., 2103, 39, 153-160; Schilder et al., Gynecol. Oncol., 2012, 127, 70-74; and Vaughan et al., Nat. Rev. Cancer, 2011, 11, 719-725). Tumors often bypass inhibitor therapies by activating alternative kinase signaling networks overcoming drug therapy (Duncan et al., Cell, 2012, 149, 307-321; and Graves et al., Biochem. J., 2013, 450, 1-8). RNAi-based studies have shown that several understudied kinases such as DDR1 or MERTK can be activated by tumors to promote resistance, supporting a role for the dark kinome in this process (Duncan et al., Cell, 2012, 149, 307-321; and Stuhlmiller et al., Cell Reports, 2015, 11, 390-404). In addition, the function of the dark kinome in HGSOC tumor growth and survival, as well as drug resistance to current therapies has not been systematically explored.

Accordingly, there is a need for development of therapies for cancer in general and for HGSOC in particular, which target a wider population of kinases and which are less likely to be rendered inefficient by the tumor resistance to kinase inhibitors.

### SUMMARY

The present disclosure provides, inter alia, multi-analyte columns for kinome isolation comprising at least four layers of multiplexer inhibitor beads, wherein: a first layer comprises a first multiplexed-inhibitor bead having at least one immobilized kinase inhibitor; a second layer comprises a second multiplexed-inhibitor bead having at least one immobilized kinase inhibitor; a third layer comprises a third multiplexed-inhibitor bead having at least one immobilized kinase inhibitor; a fourth layer comprises a fourth multiplexed-inhibitor bead having at least one immobilized kinase inhibitor; wherein the immobilized kinase inhibitor in each layer is different from the immobilized kinase inhibitor in the other layers.

The present disclosure also provides methods of generating a kinome profile of a cell comprising: obtaining a cancer cell sample from a subject; enriching protein kinases from the cancer cell-containing sample; detecting the enriched kinases; and transforming the data obtained from the detected and quantified enriched kinases into the kinome profile.

The present disclosure also provides methods of diagnosing cancer in a subject comprising: obtaining a kinome profile from the subject; and comparing the kinome profile to a standard cancer kinome profile; wherein the presence of the measured level or phosphorylation status of at least one kinase that is comparable to the cancer standard level indicates that the subject has cancer or is at risk of developing cancer.

The present disclosure also provides methods of treating cancer in a subject comprising: obtaining a cancer cell-containing sample from the subject; generating a kinome profile; comparing the kinome profile to a standard cancer kinome profile; and administering to the subject an effective amount of one or more appropriate kinase inhibitors, chemotherapeutic agents, epigenetic therapy agents, and additional biologically active compounds, or any combination thereof.

The present disclosure also provides methods of assessing a cancer therapy regimen comprising: obtaining a first cancer cell-containing sample from the subject; generating a first kinome profile; treating the subject with one or more kinase inhibitors, chemotherapy agents, or epigenetic therapies; obtaining a second cancer cell-containing sample from the subject; generating a second kinome profile; comparing the first kinome profile and the second kinome profile; and modifying the treatment regimen based on the changes in the second kinome profile as compared to the first kinome profile.

The present disclosure also provides methods for predicting the development of resistance to a chemotherapy regimen in a subject, comprising: obtaining a cancer cell-containing sample from the subject; generating a kinome profile; and comparing the kinome profile to a standard cancer kinome profile; wherein the presence of the measured level or phosphorylation status of at least one kinase that is comparable to the cancer standard level indicates that the subject is at an increased risk for development of resistance to the chemotherapy regimen.

The present disclosure also provides methods for improving effectiveness of treatment regimen for a kinase related disorder in a subject comprising: obtaining a cancer cell-containing sample from the subject; generating a kinome profile; comparing the kinome profile to a standard cancer kinome profile; and using the kinome profile to determine a more effective treatment regimen.

The present disclosure also provides methods of selecting a kinase activity modulator, the method comprising the steps of: contacting a cell, a tissue, or an organism with a compound; contacting a protein extract from the cell, the tissue, or the organism with a multi-analyte column comprising at least four layers of multiplexer inhibitor beads, wherein each layer comprises a multiplexed-inhibitor bead having at least one immobilized kinase inhibitor, and wherein the immobilized kinase inhibitor in each layer is different from the immobilized kinase inhibitor in the other layers; eluting any kinases bound to the solid supports with a denaturing agent; measuring levels of a plurality of the kinases detected to generate a kinome profile; comparing the measured kinome profile to a standard kinome profile obtained from cells that were not contacted with a compound; and using the kinome profile to select the kinase activity modulator.

The present disclosure also provides kits comprising any one or more of the multi-analyte columns described herein and instructions for use in obtaining a proteomic kinome profile.

The present disclosure also provides methods of treating High Grade Serous Ovarian Carcinoma (HGSOC) in a patient comprising administering to the patient a pharmaceutical composition comprising an effective amount of one or more therapeutic agents that inhibit at least one protein kinase, wherein the protein kinase is BRAF, MEK, ERK, FAK1, P70S6, AURKA, WEE1, CHEK1, CDK7, EphA, ATR, PI3KM/MTOR, CDK4, CDK6, IGF1R, EGFR-HER2, PAN-TK, NUAK, EIF2AK2, STK38, MRCKA, PAK4, PRPKCQ, AAK, CDK1, JAK1, ERBB4, DDR1, FGFR2, AKT2, PTK2B, MAPK1, and MAPK14.

The present disclosure also provides methods of inducing apoptosis in HGSOC cells in a patient comprising administering to the patient a pharmaceutical composition comprising an effective amount of one or more therapeutic agents that inhibit at least one protein kinase, wherein the protein kinase is MRCKA, STK38, FGFR2, ERBB4, and CHEK1.

The present disclosure also provides methods of inhibiting cell growth of HGSOC in a patient comprising administering to the patient a pharmaceutical composition comprising an effective amount of one or more therapeutic agents that inhibit at least one protein kinase, wherein the protein kinase is MRCKA, DDR1, FGFR2, ERBB4STK38, AAK1, and PLK3.

The present disclosure also provides methods of reducing cell growth in HGSOC in a patient comprising administering to the patient a pharmaceutical composition comprising an effective amount of one or more therapeutic agents that inhibit at least one protein kinase, wherein the protein kinase is PAK4, NUAK, MRCKA, FAK1, and AAK1.

The present disclosure also provides methods of suppressing carboplatin resistance in a patient having HGSOC comprising administering to the patient an effective amount of a pharmaceutical composition comprising an MRCKA inhibitor.

The present disclosure also provides a Heavy Kinome Standard comprising kinases isolated from UACC257, MOLT4, COL0205, ACHN, and PC3 cancer cell lines, wherein the kinases have been labeled using stable isotope labeled amino acids in culture (SILAC) procedure.

The present disclosure also provides methods for enriching protein kinases comprising: loading a lysate from a cancer cell-containing sample on a multi-analyte column, wherein the column comprises at least four layers of multiplexer inhibitor beads, wherein each layer comprises a multiplexed-inhibitor bead having at least one immobilized kinase inhibitor, and wherein the immobilized kinase inhibitor in each layer is different from the immobilized kinase inhibitor in the other layers; washing the multi-analyte column to remove any unbound proteins; and eluting kinases bound to the multi-analyte column with a denaturing agent.

The present disclosure also provides methods of the detection of protein kinases comprising liquid chromatography-mass spectrometry (LC-MS) incorporating selected reaction monitoring (SRM), multiple reaction monitoring (MRM), parallel reaction monitoring (PRM), tandem MS, cleavable isotopic-coded affinity tags (cICAT), or isobaric tags for absolute and relative quantification (iTRAQ).

### BRIEF DESCRIPTION OF THE DRAWINGS

The accompanying figures, which are incorporated in and constitute a part of this specification, illustrate several aspects and together with the description serve to explain the principles of the disclosure.

FIG. 1A shows application of functional proteomics to identify and validate dark kinases essential for cancer cell growth and survival. i) Combination of MIB/MS and a newly designed heavy kinome standard are used to quantify kinome within and across tumors. ii) Q-MIBs kinase signatures are then functionally interrogated for dependency and cancer function.

FIG. 1B shows design of SILAC-kinome standard. A series of cancer cell lines were selected as the SILAC “heavy” kinome standard (HKS) based on their kinome diversity and representation of the cancer kinome.

FIG. 1C shows the heavy kinome standard (HKS) containing 370 SILAC labeled kinases, representing the majority of the expressed kinome.

FIG. 1D shows the proportions of kinases quantified by Q-MIBs that are targeted by currently approved therapies, therapies in currently clinical trials and of kinases not currently targeted in cancer, many of which have no known function.

FIG. 1E shows the dark kinome consisting of untargeted and understudied kinases with sparse publications.

FIG. 1F shows characterization and quality control testing of SILAC kinome standard. Quantitation of kinase levels/activity in tumors using Q-MIBs is highly reproducible across tumor replicates and different HKS batch preparations.

FIG. 2A shows kinome profiling across tumor samples using chemical proteomics and SILAC quantitation. Cancer cell lines were selected from the NCI-60 panel based on their gene expression profile and mutation status (Gholami et al., Cell Reports, 2013, 4, 609-620). A cell line from each major cluster was selected, SILAC-labeled and kinome profiled using MIB/MS. A mixture of the 8 cell lines was used as the control to determine kinome levels. MIB log₂ ratios were determined as a ratio of SILAC-labeled cancer cell/mixture of the 8 cell lines.

FIG. 2B shows PCA analysis of cancer kinome(s) identified cell lines that exhibited the most distinct kinase signatures and those that had similar overall MIB-log₂ ratios.

FIG. 2C shows Identification of 5 SILAC cancer cell lines that provide the most coverage of the human kinome to be used as a ‘heavy’ kinome standard (HKS) for profiling kinome(s) in tumors independent of cancer type.

FIG. 2D shows SILAC labeled kinases that have FDA-approved drugs.

FIG. 2E shows SILAC labeled kinases that are in clinical trials for cancer.

FIG. 2F shows comparing Q-MIBs-determined kinase quants relative to traditional western blot methods using total and phospho-specific antibodies. Basal kinome profiling of 4 distinct HGSOC cell lines relative to HKS identifies cell-specific kinase drivers that were confirmed by western blot.

FIG. 2G shows Q-MIBs reproducibly quantifies kinome in patient tumors. Baseline kinome signature of a single colorectal tumor was assessed by Q-MIBs on 3 distinct days. Correlation plots demonstrate the high degree overlap between distinct Q-MIBs runs.

FIG. 2H shows quantitation of kinases is reproducible independent of protein concentration input. Increasing protein concentration of tumor samples were mixed 1:1 with HKS and subjected to Q-MIBs analysis. Correlation plots show quantitation is highly correlation across protein concentration inputs.

FIG. 2I shows reproducibility of the Q-MIBs assay was confirmed using a model-based approach for assessing technical reproducibility and outlier detection. A scatter plot of the coefficient of variation (CV) versus difference (D) for 300 duplicate MIB pairs for a particular Q-MIBs sample, demonstrates the reproducibility of the assay.

FIG. 3A shows application of Q-MIBs to define kinome signature of High-Grade Serous Ovarian Carcinoma (HGSOC). Differential kinase levels/activities amongst HGSOC patient/PDX tumors, cell lines and normal ovary tissue determined by Q-MIBs.

FIG. 3B shows PCA analysis of individual kinase quants across all Q-MIBs profiled samples.

FIG. 3C shows PCA analysis of kinome(s) across all Q-MIBs profiled samples.

FIG. 3D shows hierarchical clustering of Q-MIBs defined kinome(s).

FIG. 3E shows volcano plot depicting kinases that are established targets in ovarian cancer elevated or repressed in tumors relative to ovary. Statistical differences in Q-MIB-log₂ ratios of kinases comparing ovary tissues and HGSOC PDX tumors, patient tumors and cell lines were determined by ANOVA Benjamini-Hochberg adjusted p values at FDR of <0.05.

FIG. 3F shows volcano plot depicting kinases that have not been explored in ovarian cancer elevated or repressed in tumors relative to ovary. Statistical differences in Q-MIB-log₂ ratios of kinases comparing ovary tissues and HGSOC PDX tumors, patient tumors and cell lines were determined by ANOVA Benjamini-Hochberg adjusted p values at FDR of <0.05.

FIG. 3G shows heat map of Q-MIBs determined HGSOC kinome signature. Kinases that were common across HGSOC tumors and cells and statistically distinct from ovary represent the HGSOC kinome.

FIG. 3H shows Q-MIBs-defined HGSOC kinome signature is highly correlated across tumor sections. (a) A single HGSOC patient tumor was cut into 4 pieces and subjected to Q-MIBs analysis.

FIG. 3I shows correlation plots of each tumor piece were carried out and R-values calculated using Perseus.

FIG. 3J shows Heat map of Q-MIB-determined log₂ ratios across tumor pieces relative to normal ovary tissue.

FIG. 3K shows STRING analysis of the HGSOC kinome signature revealed a network of known oncogenic kinase drivers, as well as understudied kinases with no established function in HGSOC, representing potential therapeutic targets in HGSOC.

FIG. 4A shows HGSOC cell line kinome(s) differ from tumors. Volcano plot depicts elevated cell cycle-related kinases in cells relative to tumors.

FIG. 4B shows boxplot of statistically significant Q-MIB-binding log₂ ratios commonly induced or repressed relative to HKS across HGSOC cells, patient tumors and PDX tumors based on Benjamin-Hochberg adjusted p values at FDR of <0.05.

FIG. 4C shows graph of Q-MIB-determined ERBB2 log₂ ratios relative to HKS across HGSOC cell lines, patient and PDX tumors.

FIG. 4D shows protein levels of selected Q-MIBs-determine kinases in ovary tissue and HGSOC patient tumors, as determined by western blot.

FIG. 4E shows Protein levels of selected Q-MIBs-determine kinases amongst HGSOC PDX tumors, as determined by western blot.

FIG. 4F shows PhosphoPath analysis of TiO₂-determined phosphorylation profiles in HGSOC patient tumors.

FIG. 5A shows pathway activation of kinases currently targeted in clinical trials for HGSOC, as determined by western blot.

FIG. 5B shows PCA (inset) and hierarchical clustering of growth inhibition demonstrates distinct response of HGSOC cell lines to kinase inhibitors currently in clinical trials for the treatment of HGSOC. Cell viability was determined by Cell Titer Glo analysis.

FIG. 5C shows activated forms of kinases in clinical trials for other cancers but not HGSOC as shown by western blot.

FIG. 5D shows dark kinases not currently targeted in cancer and/or no established association with HGSOC, as shown by western blot.

FIG. 5E shows knockdown of HGSOC kinome signature in OVCAR4 cells. Depletion of several dark kinases inhibits cell growth to extent of kinases currently in clinical trials for HGSOC. Cells were transfected with siRNAs targeting kinases and cultured for 120 hrs. Cell viability was assessed by Cell-Titer Glo according to manufacturer. Data represent the mean of six replicates.

FIG. 5F shows RNAi-mediated knockdown of dark kinases blocks cell growth across HGSOC cell lines. Cells were transfected with siRNAs targeting kinases and cultured for 120 hrs. Cell viability was assessed by Cell-Titer Glo according to manufacturer. Data represent the mean of six replicates.

FIG. 5G shows knockdown of various dark kinases induces apoptosis amongst HGSOC cells. PARP cleavage following kinase siRNA-depletion was determined by western blot.

FIG. 6A shows genetic and mutational landscape of HGSOC cell lines used to functionally interrogate the Q-MIBs-determined HGSOC tumor kinome signature.

FIG. 6B shows RNA levels of dark kinases following targeted siRNA knockdown that have no established antibody were determined by qRT-PCR.

FIG. 6C shows validation of siRNA knockdown of kinases explored in siRNA knockdown screens, as determined by blot.

FIG. 6D shows RNAi-mediated knockdown of kinases blocks cell growth across HGSOC cell lines. Cells were transfected with siRNAs targeting kinases and cultured for 120 hrs. Cell viability was assessed by Cell-Titer Glo according to manufacturer. Data represent the mean of six replicates.

FIG. 6E shows HGSOC cells treated with PAK4 inhibitor PF-03758309. Cells were treated with increasing concentrations of PF-03758309 and cell viability assessed by Cell Titer Glo according to manufacturer's instructions. Data represent the mean of six replicates.

FIG. 6F targeted spectrum in AAK1 gene of 2 distinct siRNAs.

FIG. 6G shows RNA levels of AAK1 in OVCAR4 cells following treatment with 2 distinct siRNAs.

FIG. 6H shows AAK1 knockdown with 2 distinct siRNAs inhibit cell growth of OVCAR4 cells. Cells were treated with AAK1 siRNAs for 48 hours and cell viability assessed by Cell Titer Glo.

FIG. 6I shows AAK1 knockdown inhibiting established substrate phosphorylation of AP2M1 and reducing MYC, pERK and Cyclin D1 protein levels. OVCAR4 cells were treated with AAK1 siRNAs for 48 hours and protein levels assessed by western blot.

FIG. 6J shows AAK1 knockdown inhibiting established substrate phosphorylation of AP2M1 and reducing MYC, pERK and Cyclin D1 protein levels. OVCAR4 cells were treated with AAK1 siRNAs for 8, 24 or 48 hours and protein levels assessed by western blot.

FIG. 6K shows targeted spectrum in MRCKA gene of 2 distinct siRNAs.

FIG. 6L shows MRCKA knockdown with 2 distinct siRNAs inhibiting cell growth and inducing apoptosis in OVCAR4 cells. Cells were treated with MRKCA siRNAs for 48 hours and cell viability assessed by Cell Titer Glo and apoptosis determined by PARP blot.

FIG. 6M shows MRCKA knockdown with 2 distinct siRNAs inhibits cell growth and induces apoptosis in KURAMOCHI cells. Cells were treated with MRKCA siRNAs for 48 hours and cell viability assessed by Cell Titer Glo and apoptosis determined by PARP blot.

FIG. 7A shows proteomic characterization of MRCKA knockdown reveals a role for MRCKA in a variety of oncogenic signaling pathways in HGSOC cells. Application of KASPR to define biomarkers of MRKCA depletion in HGSOC cells. OVCAR4 cells were treated with control or MRCKA siRNAs and subjected to kinome profiling using Q-MIBs, phosphoproteomics and single run proteome analysis.

FIG. 7B shows MRCKA reprograms kinome(s), phosphoproteome and total proteomes, as shown by correlation plots.

FIG. 7C shows volcano plot depicting kinases induced or repressed following MRCKA knockdown. OVCAR4 cells were transfected with control or MRCKA siRNA for 48 hours and subjected to Q-MIBs analysis. Statistical differences in Q-MIB-log 2 ratios of kinases comparing MRCKA relative to control siRNA were determined by ANOVA Benjamini-Hochberg adjusted p values at FDR of <0.05.

FIG. 7D shows heat map depicting kinases induced or repressed following MRCKA knockdown. OVCAR4 cells were transfected with control or MRCKA siRNA for 48 hours and subjected to Q-MIBs analysis. Statistical differences in Q-MIB-log₂ ratios of kinases comparing MRCKA relative to control siRNA were determined by ANOVA Benjamini-Hochberg adjusted p values at FDR of <0.05.

FIG. 7E shows STRING analysis of Q-MIBs determined kinases repressed following MRCKA knockdown.

FIG. 7F shows volcano plot depicts changes phosphorylation following MRCKA knockdown. OVCAR4 cells were transfected with control or MRCKA siRNA for 48 hours and subjected to global phosphoproteomics evaluation using TiO₂ enrichment. Statistical differences in log₂ ratios of kinase phosphopeptides comparing MRCKA relative to control siRNA treated cells were determined by ANOVA Benjamini-Hochberg adjusted p values at FDR of <0.05.

FIG. 7G shows Phosphopath analysis of global phosphorylation repressed by MRCKA knockdown for 48 hours in OVCAR4 cells.

FIG. 7H shows KEGG analysis of protein levels reduced in MRCKA treated cells.

FIG. 7I shows MRCKA knockdown inhibiting FAK1, PAK4, AURKA and CHEK1 signaling, as well as induced PAK1 activity in OVCAR4 cells, as determined by blot.

FIG. 8A shows proteomic analysis of MRCKA knockdown in HGSOC cells using KASPR. Hierarchical clustering of Q-MIBs-determined kinome(s) following MRCKA knockdown for 48 hours in OVCAR4 cells.

FIG. 8B shows global phosphoproteomic profiles following MRCKA knockdown for 48 hours in OVCAR4 cells.

FIG. 8C shows single-run proteomes following MRCKA knockdown for 48 hours in OVCAR4 cells.

FIG. 8D shows heat map of Q-MIBs kinases induced following MRCKA knockdown in OVCAR4 cells.

FIG. 8E shows predicted kinases activated in MRCKA knockdown cells using Kinase Substrate Enrichment Analysis of phosphoproteomics datasets.

FIG. 8F shows volcano plot of single-run proteome analysis of OVCAR4 cells following MRCKA knockdown.

FIG. 8G shows reduced FAK1, PAK4 and CHEK1 protein levels observed following MRKCA using 2 distinct siRNAs in KURAMOCHI cells.

FIG. 9A shows FAK1 activating phosphorylation and total FAK1 levels are reduced by MRCKA knockdown in HGSOC cells as shown by western blot.

FIG. 9B shows MRCKA knockdown reducing focal adhesion signaling. COV362 cells were transfected with MRCKA or control siRNAs for 24, 48 or 72 hours and subjected to western blot analysis.

FIG. 9C shows depletion of MRCKA inhibits components of actin remodeling, cell cycle kinases and induces PAK1 signaling in HGSOC cell as shown by blot.

FIG. 9D shows MRCKA knockdown reduces cell migration. COV362 cells were transfected with MRCKA or control siRNAs and migration monitored over a 24 hour period using the xCELLigence Real-Time Cell Analyzer (RTCA).

FIG. 9E shows PAK1 activation following MRCKA knockdown. OVCAR4 cells were transfected with MRCKA or control siRNAs and PAK1/2 phosphorylation determined by blot.

FIG. 9F shows PAK1 activation following MRCKA knockdown. COV362 cells were transfected with MRCKA or control siRNAs and PAK1/2 phosphorylation determined by blot.

FIG. 9G shows cycle cell arrest occurs following MRCKA knockdown. OVCAR4 cells were transfected with MRCKA or control siRNAs for 24, 48 or 72 hours and subjected to western blot analysis for cell cycle markers.

FIG. 9H shows knockdown of MRCKA arrests cells in G1/S phase, as shown by FACS analysis.

FIG. 9I shows proposed function of MRCKA in HGSOC. MRCKA promotes cell cycle, survival, actin remodeling and focal adhesion signaling.

FIG. 10 shows that MRCKA knockdown does not influence FAK1 transcription as determined by qRT-PCR. Data presented are triplicate experiments SEM. *p≤0.05 by student's t-test.

FIG. 11A shows depletion of MRCKA enhancing growth inhibitory effects of carboplatin, PARP inhibitors, BET bromodomain inhibitors and kinase inhibitors, as shown by heat map. OVCAR4 cells were transfected with control nontargeting or dark kinase siRNAs and treated with escalating doses of appropriate drugs.

FIG. 11B shows knockdown of MRCKA sensitizing HGSOC cells to carboplatin. Cells transfected with control or MRKCA siRNAs were treated with increasing doses of carboplatin. Cell viability was assessed by Cell Titer Glo according to manufacturer's instructions. Data represent the mean of six replicates. GR50 were determined using PRISM.

FIG. 11C shows depletion of MRCKA from HGSOC cells increasing apoptosis following carboplatin treatment, as determined by cleaved PARP blots.

FIG. 11D shows knockdown of MRCKA sensitizing HGSOC cells to PARP inhibition. Cells transfected with control or MRKCA siRNAs were treated with increasing doses of olaparib. Cell viability was assessed by Cell Titer Glo according to manufacturer's instructions. Data represent the mean of six replicates. GR50 were determined using PRISM.

FIG. 11E shows depletion of MRCKA from HGSOC cells increasing apoptosis following olaparib treatment, as determined by cleaved PARP blots.

FIG. 11F shows RNA-mediated knockdown of MRCKA enhancing growth inhibition following WEE1 inhibition Cells transfected with control or MRKCA siRNAs were treated with increasing doses of MK-1775 or JQ1. Cell viability was assessed by Cell Titer Glo according to manufacturer's instructions. Data represent the mean of six replicates. GR50 were determined using PRISM.

FIG. 11G shows RNA-mediated knockdown of MRCKA enhancing growth inhibition following or BET bromodomain inhibition. Cells transfected with control or MRKCA siRNAs were treated with increasing doses of MK-1775 or JQ1. Cell viability was assessed by Cell Titer Glo according to manufacturer's instructions. Data represent the mean of six replicates. GR50 were determined using PRISM.

FIG. 12A shows knockdown of AAK1 sensitizing HGSOC cells to BET bromodomain inhibition. Cells transfected with control or AAK1A siRNAs were treated with increasing doses of JQ1. Cell viability was assessed by Cell Titer Glo according to manufacturer's instructions. Data represent the mean of six replicates. GR50 were determined using PRISM.

FIG. 12B shows knockdown of MRCKA sensitizes HGSOC cells to WEE1 inhibition. Cells transfected with control or MRCKA siRNAs were treated with increasing doses of MK-1775. Cell viability was assessed by Cell Titer Glo according to manufacturer's instructions. Data represent the mean of six replicates. GR50 were determined using PRISM.

FIG. 13A shows kinome map of BDP5290 targets as determined by radioisotope based 33P assay. Inhibition analysis of BDP5290 at 10 μM was carried out on 371 kinases at Reaction Biology Corporation. Red circles indicate % of kinase activity inhibited by BDP5290.

FIG. 13B shows hierarchical clustering of MIB-determined kinome(s) treated with various small molecules. OVSAHO cells were treated with inhibitors at a dose of 2 μM for 4 h and subjected to MIB/MS.

FIG. 13C shows MIB-based kinome profiling identifies candidate MRCKA inhibitors. OVSAHO cells were treated with inhibitors at a dose of 2 μM for 4 hours and kinase inhibition profiles determined by MIB/MS. Bar graph depicts % of kinase bound to MIBs relative to DMSO control as determined by label free quantitation.

FIG. 13D shows MIB-determined kinome inhibition profiles of candidate MRCKA inhibitors. OVSAHO cells were treated with 2 μM of inhibitor for 4 hours and analyzed by MIB/MS. Scatterplot depicts difference in kinase MIB-binding of drug treated relative to control DMSO as determined by Students T-test.

FIG. 13E shows treatment of cells with A-674563 MRCKA recapitulating MRCKA knockdown signaling. COV362 cells were treated with 0.5, 1 or 3 μM of A-674563 or SB-772077 for 48 hours and signaling monitored by western blot.

FIG. 13F shows treatment of cells with A-674563 of MRCKA blocking cell migration. COV362 cells were treated with 1 μM of A-674563 or DMSO and migration monitored over a 24 hour period using the xCELLigence Real-Time Cell Analyzer (RTCA).

FIG. 13G shows A-674563 treatment reducing cell viability. COV362 cells were treated with increasing concentrations of A-674563 and cell viability assessed by Cell Titer Glo according to manufacturer's instructions. Data represent the mean of six replicates.

FIG. 14A shows BDP5290 inhibiting HGSOC cell viability at higher concentrations. Cell viability was assessed by Cell Titer Glo according to manufacturing. Data represent the mean of six replicates.

FIG. 14B shows long-term colony formation analysis of BDP5290 treated HGSOC cells. Cells were treated with 0.5, 1 or 3 μM of BDP5290 for 14 days and colony formation determined using crystal violet staining.

FIG. 14C shows treatment of OVCAR4 cells with increasing doses of BDP5290. HGSOC cells were treated with 0.5, 1 or 3 μM of BDP5290 for 48 hours and signaling analyzed by western blot.

FIG. 14D shows BDP5290 treatment of HGSOC cell lines. HGSOC cells were treated with 0.5, 1 or 3 μM of BDP5290 for 48 hours and signaling analyzed by western blot.

FIG. 14E shows kinome profiling of BDP5290 treated KURAMOCHI cells by MIB/MS. PCA plot of MIB-determined SILAC ratios. Cells were treated with 2 μM of BDP5290 or DMSO for 4 hours and kinome alterations determined by MIBs.

FIG. 14F shows BDP5290 treatment inhibiting AURKB but not MRCKA or MRCK in OVCAR 4 cells. Cells were treated with 2 μM BDP5290 for 24 hours and kinome profiled using MIB/MS. Volcano plot depicts LFQ quantitation as a ratio of BDP5290/DMSO. Statistical kinases are identified using Students T-tests with FDR 0.05.

FIG. 14G shows increasing doses of BDP5290 elute AURKB but not MRCKB from MIB beads as shown by western blot. Cell lysates were incubated with various doses of BDP5290 for 30 minutes followed by incubation with MIB-beads. MIB-elutions were then analyzed by western blot.

FIG. 14H shows AURKB is eluted from MIB-beads but MRCK is not as determined by MIB/MS. Cell lysates were incubated with various doses of BDP5290 for 30 minutes followed by incubation with MIB-beads. Kinome elutions were then analyzed using LC-MS/MS and changes in kinase binding quantified using label free quantitation.

FIG. 14I shows small molecules from PKIS library that inhibit MRCKA.

FIG. 14J shows kinome inhibition profiles of BDP5290, trametinib, lapatinib or SD-208. Scatter plot depicts statistical differences in MIB-binding as a ratio of inhibitor/DMSO. Cells were treated with inhibitors at a dose of 2 μM and subjected to MIB/MS. Changes in MIB-binding were quantified using label free quantitation. Data presented in (A) are triplicate experiments SEM. *p≤0.05 by student's t-test.

Additional advantages of the disclosure will be set forth in part in the description which follows, and in part will be apparent from the description, or can be learned by practice of the embodiments disclosed herein. The advantages of the disclosure can be realized and attained by means of the elements and combinations particularly pointed out in the appended claims. It is to be understood that both the foregoing general description and the following detailed description are exemplary and explanatory only and are not restrictive of the embodiments, as claimed.

### DESCRIPTION OF EMBODIMENTS

The terminology used herein is for the purpose of describing particular embodiments only and is not intended to be limiting. Various terms relating to aspects of disclosure are used throughout the specification and claims. Such terms are to be given their ordinary meaning in the art, unless otherwise indicated. Other specifically defined terms are to be construed in a manner consistent with the definitions provided herein.

Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is in no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

As used herein, the singular forms “a,” “an” and “the” include plural referents unless the context clearly dictates otherwise.

As used herein, the terms “subject” and “patient” are used interchangeably. A subject may include any animal, including mammals. Mammals include, without limitation, farm animals (e.g., horse, cow, pig), companion animals (e.g., dog, cat), laboratory animals (e.g., mouse, rat, rabbits), and non-human primates. In some embodiments, the subject is a human.

The present disclosure provides multi-analyte columns comprising at least four layers of multiplexer inhibitor beads wherein each layer comprises a solid support comprising at least one immobilized kinase inhibitor with specific kinase binding affinity, wherein the immobilized kinase inhibitor in each layer is different from the immobilized kinase inhibitor in the other layers.

As used herein, the term “solid support” means and includes any support capable of binding the affinity ligands disclosed herein. Well-known supports or carriers include glass, polystyrene, polypropylene, polyethylene, dextran, nylon, amylases, natural and modified celluloses, polyacrylamides, and magnetite. The support material may have virtually any possible structural configuration so long as the coupled affinity ligand is capable of binding to kinases. Thus, the support configuration may be spherical, as in a bead, or cylindrical, as in the inside surface of a test tube, or the external surface of a rod. Alternatively, the surface may be flat such as a sheet, test strip, etc. In one non-limiting embodiment, the solid support may be sepharose (e.g. EAH Sepharose 4B or ECH Sepharose 4B) or polystyrene beads. Those skilled in the art will know many other suitable carriers for affinity binding a protein, or will be able to ascertain the same by use of routine experimentation.

As used herein, the term “inhibitor” refers to inhibitory molecules identified using in vitro and in vivo assays of the expression of genes hyper- or hypo-methylated in a kinase related disorder, mutations associated with a kinase related disorder, or the translation proteins encoded thereby. Inhibitors also include naturally occurring and synthetic ligands, antagonists, agonists, antibodies, peptides, cyclic peptides, nucleic acids, antisense molecules, ribozymes, shRNAs, RNAi molecules, small organic molecules and the like. Such assays for inhibitors and activators include, e.g., (1)(a) the mRNA expression, or (b) proteins expressed by genes hyper- or hypomethylated in a kinase related disorder in vitro, in cells, or cell extracts; (2) applying putative modulator compounds; and (3) determining the functional effects on activity, as described herein.

Assays comprising in vivo measurement of a kinase related disorder, or genes hyper- or hypomethylated in a kinase related disorder are treated with a potential activator, inhibitor, or modulator are compared to control assays without the inhibitor, activator, or modulator to examine the extent of inhibition. Controls (untreated) are assigned a relative activity value of 100%. Inhibition of gene expression, protein expression associated with a kinase related disorder is achieved when the activity value relative to the control is about 80%, about 50%, or about 0-25%. Activation of gene expression, or proteins associated with a kinase related disorder is achieved when the activity value relative to the control (untreated with activators) is about 110%, about 150%, about 200%, about 300%, about 400%, about 500% (i.e., two to five fold higher relative to the control), or about 1000%, or about 2000%, or about 3000%, or greater.

There are over 500 known human kinases at the present (Manning et al., Science, 2002, 298, 1912-1934; Subramani et al., ISRN Computational Biology, 2013, Article ID 417634; and Milanesi et al., BMC Bioinformatics, 2005, 6, S20). In some embodiments, the protein kinases of the present disclosure include, without limitation, ABL1, AAK1, ABL1, ABL2, ACK1, ACVR1C, ACVR2A, ACVR2B, ADCK1, ADCK3, ADCK4, ADCK5, AKT1, AKT2, AKT3, ALK1/ACVRL1, ALK2/ACVR1, ALK3/BMPR1A, ALK4/ACVR1B, ALK5/TGFBR1, ALK6/BMPR1B, ARAF, ARK5/NUAK1, ASK1/MAP3K5, ATM, ATR, AURKA, AURKB, Aurora A, Aurora B, Aurora C, AXL, BLK, BMP2K, BMPR2, BMX, BRAF, BRD3, BRD4, BRK, BRSK1, BRSK2, BTK, BUB1, BUB1B, CAMK1, CAMK1a, CAMK1b, CAMK1d, CAMK1g, CAMK2a, CAMK2b, CAMK2d, CAMK2g, CAMK4, CAMKK1, CAMKK2, CAMKV, CASK, CDCl42BPG, CDCl7, CDK1, CDK10, CDK11A, CDK11B, CDK12, CDK13, CDK14, CDK15, CDK16, CDK17, CDK18, CDK19, CDK2, CDK3, CDK4, CDK5, CDK6, CDK7, CDK8, CDK9, CDKL3, CDKL5, CHEK1, CHEK2, CHK1, CHK2, CK1a1, CK1a1L, CK1d, CK1epsilon, CK1g1, CK1g2, CK1g3, CK2a, CK2a2, c-Kit, CLK1, CLK2, CLK3, CLK4, c-MER, c-MET, COT1/MAP3K8, CSF1R, CSK, CSNK1A1, CSNK1A1L, CSNK1D, CSNK1E, CSNK1G1, CSNK1G2, CSNK1G3, CSNK2A1, CSNK2A2, c-Src, CTK/MATK, DAPK1, DAPK2, DCAMKL1, DCAMKL2, DCLK1, DCLK3, DDR1, DDR2, DLK/MAP3K12, DMPK, DMPK2, DRAK1/STK17A, DSTYK, DYRK1A, DYRK1B, DYRK2, DYRK3, DYRK4, EEF2K, EGFR, EIF2AK1, EIF2AK2, EIF2AK3, EIF2AK4, EPHA1, EPHA2, EPHA3, EPHA4, EPHA5, EPHA6, EPHA7, EPHA8, EPHB1, EPHB2, EPHB3, EPHB4, EPHB6, ERBB2, ERBB2/HER2, ERBB4, ERBB4/HER4, ERK1, ERK2/MAPK1, ERK5/MAPK7, ERK7/MAPK15, ERN1, ERN1/IRE1, ERN2/IRE2, FAK/PTK2, FER, FES/FPS, FGFR1, FGFR2, FGFR3, FGFR4, FGR, FLT1/VEGFR1, FLT3, FLT4/VEGFR3, FMS, FRK, FYN, GAK, GCK/MAP4K2, GLK/MAP4K3, GRK1, GRK2, GRK3, GRK4, GRK5, GRK6, GRK7, GSK3a, GSK3b, Haspin, HCK, HGK/MAP4K4, HIPK1, HIPK2, HIPK3, HIPK4, HPK1/MAP4K1, IGF1R, IKBKB, IKBKE, IKKa/CHUK, IKKb/IKBKB, IKKe/IKBKE, ILK, INSR, IR, IRAK1, IRAK3, IRAK4, IRR/INSRR, ITK, JAK1, JAK2, JAK3, JNK1, JNK2, JNK3, KDR/VEGFR2, KHS/MAP4K5, KIT, KSR1, KSR2, LATS1, LATS2, LCK, LCK2/ICK, LIMK1, LIMK2, LKB1, LMTK2, LOK/STK10, LRRK2, LYN, LYN B, MAK, MAP2K1, MAP2K2, MAP2K3, MAP2K4, MAP2K5, MAP2K6, MAP3K1, MAP3K11, MAP3K12, MAP3K2, MAP3K3, MAP3K4, MAP3K6, MAP3K7, MAPK10, MAPK3, MAPK6, MAPK7, MAPK8, MAPK9, MAPKAPK2, MAPKAPK3, MAPKAPKS/PRAK, MARK1, MARK2/PAR-1Ba, MARK3, MARK4, MAST1, MAST2, MAST3, MAST4, MASTL, MEK1, MEK2, MEK3, MEK5, MEKK1, MEKK2, MEKK3, MEKK6, MELK, MERTK, MET, MINK/MINK1, MKK4, MKK6, MKK7, MKNK1, MKNK2, MLCK/MYLK, MLCK2/MYLK2, MLK1/MAP3K9, MLK2/MAP3K10, MLK3/MAP3K11, MLK4, MLKL, MLTK, MNK1, MNK2, MRCKa/CDCl42BPA, MRCKb/CDCl42BPB, MSK1/RPS6KA5, MSK2/RPS6KA4, MSSK1/STK23, MST1/STK4, MST2/STK3, MST3/STK24, MST4, MTOR, MUSK, MYLK, MYLK3, MYLK4, MYO3A, MYO3b, NEK1, NEK11, NEK2, NEK3, NEK4, NEK5, NEK6, NEK7, NEK8, NEK9, NIM1, NLK, NRBP1, NRK, OBSCN, OSR1/OXSR1, P38a/MAPK14, P38b/MAPK11, P38d/MAPK13, P38g, p70S6K/RPS6KB1, p70S6Kb/RPS6KB2, PAK1, PAK2, PAK3, PAK4, PAK5, PAK6, PAK7, PASK, PBK/TOPK, PDGFRa, PDGFRb, PDIK1L, PDK1/PDPK1, PDK3, PDPK1, PDPK2, PEAK1, PHKg1, PHKg2, PI4K2A, PI4K2B, PI4KA, PI4 KB, PIK3C2A, PIK3C3, PIK3CB, PIK3CD, PIK3CG, PIK3R1, PIK3R2, PIK3R4, PIK4CA, PIKFYVE, PIM1, PIM2, PIM3, PIP4K2A, PIP4K2B, PIP4K2C, PIP5K1A, PIP5K1B, PIP5K1C, PIP5K3, PKA, PKAcb, PKAcg, PKCa, PKCb1, PKCb2, PKCd, PKCepsilon, PKCeta, PKCg, PKCiota, PKCmu/PRKD1, PKCnu/PRKD3, PKCtheta, PKCzeta, PKD2/PRKD2, PKG1a, PKG1b, PKG2/PRKG2, PKMYT1, PKN1/PRK1, PKN2/PRK2, PKN3/PRK3, PLK1, PLK2, PLK3, PLK4/SAK, POMK, PRKAA1, PRKAA2, PRKACA, PRKACB, PRKACG, PRKCA, PRKCB, PRKCD, PRKCE, PRKCG, PRKCH, PRKCI, PRKCQ, PRKDC, PRKG1, PRKX, PRKY, PRPF4B, PTK2B, PTK6, PTK7, PYK2, RAF1, RET, RIOK1, RIOK2, RIPK1, RIPK2, RIPK3, RIPK4, RIPK5, ROCK1, ROCK2, RON/MST1R, ROR2, ROS/ROS1, RPS6KA1, RPS6KA2, RPS6KA3, RPS6KA6, RSK1, RSK2, RSK3, RSK4, SBK1, SCYL1, SGK1, SGK196, SGK2, SGK223, SGK3/SGKL, SGK494, SIK1, SIK2, SIK3, SLK, SLK/STK2, SNARK/NUAK2, SNRK, SRC, SRMS, SRPK1, SRPK2, SSTK/TSSK6, STK11, STK16, STK17B, STK21/CIT, STK22D/TSSK1, STK25, STK25/YSK1, STK26, STK32B, STK32B/YANK2, STK32C, STK32C/YANK3, STK33, STK35, STK36, STK38/NDR1, STK38L/NDR2, STK39/STLK3, STK40, STRADA, STRADB, SYK, TAK1, TAOK1, TAOK2/TAO1, TAOK3/JIK, TBK1, TEC, TESK1, TESK2, TGFBR2, TIE2/TEK, TLK1, TLK2, TNIK, TNK1, TNK2, TP53RK, TRIM28, TRIM33, TRIO, TRKA, TRKB, TRKC, TRRAP, TSSK2, TSSK3/STK22C, TSSK4, TTBK1, TTBK2, TTK, TTN, TXK, TYK1/LTK, TYK2, TYRO3/SKY, ULK1, ULK2, ULK3, ULK4, VRK1, VRK2, WEE1, WNK1, WNK2, WNK3, WNK4, YES/YES1, YSK4/MAP3K19, ZAK, ZAK/MLTK, ZAP70, and/or ZIPK/DAPK3, or any combination thereof. In some embodiments, the protein kinases of the present disclosure include any protein kinase known in the art.

Numerous kinase inhibitors are also known in the art and described in, for example, Davis et al., Nat. Biotechnol., 2011, 29, 1046; Dhaliwal et al., J. Pharmacol. Exp. Ther., 2009, 330, 334; Elkins et al., Nat. Biotechnol., 2015, 34, 95; Homan et al., ACS Chem. Biol., 2015, 10, 310-319; Luo et al., Mol. Cancer Ther., 2005, 4, 977; Patel et al., Cancer Res., 2012, 72, 5025; U.S. Pat. Nos. 9,629,851, 9,758,511, 9,963,680, 9,932,641, 9,943,609, and 9,737,535; and U.S. Patent Application Publication 2014/0243239.

In some embodiments, the kinase inhibitors include, without limitation, bisindolymaleimide-X, GW-572016, SB203580, CTx-0249885, 2,4-diaminopyrimidine, pyrazole, PP58, purvalanol B, VI16832, AX14596, SU6668, dasatinib, lapatinib, afatinib, axitinib, bosutinib, ceritinib, cobimetinib, crizotinib, entrectinib, erlotinib, fostamatinib, gefitinib, ibrutinib, imatinib, lenvatinib, mubritinib, nilotinib, pazopanib, ruxolitinib, sorafenib, sunitinib, SU6656, vandetanib, vemurafenib, A47, CHEMBL592030, CHEMBL1993661, CHEMBL1983268, KCB-A41, AZD3463, FRAX486, PF-06463922, GDL-0941, THZ1, JQ1, MK1775, AZD7762, BDP5290, RKI-1447, SP772077, GSK46317A, A647563, BDP5290, RKI-1447, SP772077, GSK46317A, PF-3758309, PND1186, and/or PP121, or any combination thereof. In some embodiments, the kinase inhibitors of the present disclosure include any kinase inhibitors known in the art.

In some embodiments, the kinase inhibitors are immobilized on the solid support. A wide variety of appropriate coupling methods may be used to attach the affinity ligands to a solid support. The coupling may be performed with covalent linkages such as amide linkages (e.g., amino NETS-ester), ester bonds, phosphoester bonds, or disulfide bonds. The coupling may also be performed using methods such as affinity tags, such as antigenic tags or other binding methods (e.g., antibody-protein A; biotin-streptavidin; FLAG-tag (Sigma-Aldrich, Hopp et al., Nat. Biotech., 1988, 6, 1204-1210); glutathione S-transferase (GST)/glutathione; hemagluttanin (HA) (Wilson et al., Cell, 1984, 37, 767); intein fusion expression systems (New England Biolabs, USA) Chong et al., Gene, 1997, 192, 271-281; maltose-binding protein (MBP)); poly His-(Ni or Co) (Gentz et al., Proc. Nat'l. Acad. Sci. USA, 1989, 86, 821-824); or thiol-gold. Fusion proteins containing GST-tags at the N-terminus of the protein are also described in U.S. Pat. No. 5,654,176. Magnetic separation techniques may also be used such as Strepavidin-DynaBeads® (Life Technologies, USA). Alternatively, photo-cleavable linkers may be used, e.g., U.S. Pat. No. 7,595,198. A wide variety of coupling methods, including polystyrene affinity peptides, are reviewed by Nakanishi et al., Curr. Proteomics, 2008, 5, 161-175. Many other systems are known in the art and are suitable for use with the present disclosure.

Each kinase inhibitor of the present disclosure may be specific for a single kinase or for multiple kinases. Multi-specific kinase inhibitors are well known in the art. For example, KCB-A41 binds at least 13 kinases with moderate to high affinity, while PP58 and FRAX46 each bind different but overlapping sets of over 30 kinases with moderate to high affinity. In some embodiments, the kinase inhibitor is specific for two or more kinases. In some embodiments, the kinase inhibitor is specific for five or more kinases. In some embodiments, the kinase inhibitor is specific for 10 or more kinases. In some embodiments, the kinase inhibitor is specific for 20 or more kinases. In some embodiments, the kinase inhibitor is specific for 50 or more kinases.

The term, “moderate affinity”, as used herein, refers to any ligand (e.g., a kinase inhibitor) that attaches to another molecule (e.g., a kinase) having a micromolar K_{D}. The term, “high affinity”, as used herein, refers to any ligand (e.g. a kinase inhibitor) that attaches to another molecule (e.g., a kinase) having a submicromolar K_{D}. For example, a high affinity ligand may have an affinity residing in the nanomolar range. Alternatively, a high affinity ligand may have an affinity residing in the picomolar range.

The present disclosure also provides immobilizing one or more kinase inhibitors on the beads. In some embodiments, there is a single kinase inhibitor immobilized a bead. In some embodiments, there are at least two different kinase inhibitors immobilized on the same bead. In some embodiments, there are at least three different kinase inhibitors immobilized on the same bead. In some embodiments, there are at least four different kinase inhibitors immobilized on the same bead. In some embodiments, there are at least five different kinase inhibitors immobilized on the same bead. In some embodiments, there are more than five different kinase inhibitors immobilized on the same bead.

The present disclosure also provides multi-analyte kinase affinity columns comprising multiple kinase inhibitors immobilized on a solid support, such as beads. In some embodiments, the beads carrying different sets of kinase inhibitors are intermixed within the column. In some embodiments, the beads carrying different sets of kinase inhibitors are arranged in layers within the column. In some embodiments, the column comprises at least two layers of beads, wherein each bead carries a distinct set of kinase inhibitors. In some embodiments, the column comprises at least three layers of beads, wherein each bead carries a distinct set of kinase inhibitors. In some embodiments, the column comprises at least four layers of beads, wherein each bead carries a distinct set of kinase inhibitors. In some embodiments, the column comprises at least five layers of beads, wherein each bead carries a distinct set of kinase inhibitors. In some embodiments, the column comprises at least six layers of beads, wherein each bead carries a distinct set of kinase inhibitors. In some embodiments, the column comprises at least seven layers of beads, wherein each bead carries a distinct set of kinase inhibitors. In some embodiments, the column comprises at least eight layers of beads, wherein each bead carries a distinct set of kinase inhibitors. In some embodiments, the column comprises at least nine layers of beads, wherein each bead carries a distinct set of kinase inhibitors. In some embodiments, the column comprises at least ten layers of beads, wherein each bead carries a distinct set of kinase inhibitors. In some embodiments, the column comprises more than ten layers of beads, wherein each bead carries a distinct set of kinase inhibitors. The kinase inhibitor sets immobilized on the beads within each layer may be wholly distinct, or may contain overlapping kinase inhibitor species. In some embodiments, all the layers within the multi-analyte kinase affinity column of the present disclosure comprise the same type of beads (e.g., sepharose). In some embodiments, the different layers within the multi-analyte kinase affinity column of the present disclosure comprise different types of beads (e.g., sepharose and polystyrene). The layers comprising different bead types may be arranged in any order.

In some embodiments, the multi analyte columns bind at least 50% of all known protein kinases with moderate to high affinity. In some embodiments, the multi analyte columns bind at least 60% of all known protein kinases with moderate to high affinity. In some embodiments, the multi analyte columns bind at least 70% of all known protein kinases with moderate to high affinity. In some embodiments, the multi analyte columns bind at least 80% of all known protein kinases with moderate to high affinity. In some embodiments, the multi analyte columns bind at least 90% of all known protein kinases with moderate to high affinity. In some embodiments, the multi analyte columns bind 100% of all known protein kinases with moderate to high affinity.

In some embodiments, the multi analyte columns bind at least 300 known protein kinases with moderate to high affinity. In some embodiments, the multi analyte columns bind at least 350 known protein kinases with moderate to high affinity. In some embodiments, the multi analyte columns bind at least 400 known protein kinases with moderate to high affinity. In some embodiments, the multi analyte columns bind at least 450 known protein kinases with moderate to high affinity. In some embodiments, the multi analyte columns bind at least 500 known protein kinases with moderate to high affinity. In some embodiments, the multi analyte columns bind more than 500 known protein kinases with moderate to high affinity.

The present disclosure also provides multi-analyte columns comprising at least four layers of beads, wherein each bead carries at least one immobilized kinase inhibitor, wherein the immobilized kinase inhibitor within each layer is different from the immobilized kinase inhibitors in the other layers. In some embodiments, the multi-analyte column comprises a first layer that comprises a first multiplexed-inhibitor bead having at least one immobilized kinase inhibitor; a second layer that comprises a second multiplexed-inhibitor bead having at least one immobilized kinase inhibitor; a third layer that comprises a third multiplexed-inhibitor bead having at least one immobilized kinase inhibitor; and a fourth layer that comprises a fourth multiplexed-inhibitor bead having at least one immobilized kinase inhibitor; wherein the immobilized kinase inhibitor in each layer is different from the immobilized kinase inhibitor in the other layers. In some embodiments, the multi-analyte column comprises a first layer that comprises a first multiplexed-inhibitor bead having at least one immobilized kinase inhibitor; a second layer that comprises a second multiplexed-inhibitor bead having at least one immobilized kinase inhibitor; a third layer that comprises a third multiplexed-inhibitor bead having at least one immobilized kinase inhibitor; a fourth layer that comprises a fourth multiplexed-inhibitor bead having at least one immobilized kinase inhibitor; a fifth layer that comprises a fifth multiplexed-inhibitor bead having at least one immobilized kinase inhibitor; a sixth layer that compress a sixth multiplexed-inhibitor bead having at least one immobilized kinase inhibitor; and a seventh layer that comprises a seventh multiplexed-inhibitor bead having at least one immobilized kinase inhibitor; wherein the immobilized kinase inhibitor in each layer is different from the immobilized kinase inhibitor in the other layers.

The present disclosure also provides multi-analyte columns comprising a first layer comprising a first multiplexed-inhibitor bead carrying immobilized purvalanol B; a second layer comprising a second multiplexed-inhibitor bead carrying immobilized PP58; a third layer comprising a third multiplexed-inhibitor bead carrying immobilized VI16832; and a fourth layer comprising a fourth multiplexed-inhibitor bead carrying immobilized CTx-0249885. In some embodiments, the first, second, third, and fourth layers are arranged sequentially top to bottom in the order first layer (top)-second layer-third layer-fourth layer (bottom). In some embodiments, the first, second, third, and fourth layers are arranged sequentially top to bottom in the order first layer (top)-second layer-third layer-fourth layer (bottom). In some embodiments, the four layers can be arranged in any order.

The present disclosure also provides multi-analyte columns comprising a first layer comprising a first multiplexed-inhibitor bead carrying immobilized gefitinib; a second layer comprising a second multiplexed-inhibitor bead carrying immobilized bisindolymaleimide-X; a third layer comprising a third multiplexed-inhibitor bead carrying immobilized SB203580; a fourth layer comprising a fourth multiplexed-inhibitor bead carrying immobilized dasatinib; a fifth layer comprising a fifth multiplexed-inhibitor bead carrying immobilized purvalanol B; a sixth layer comprising a sixth multiplexed-inhibitor bead carrying immobilized PP58; and a seventh layer comprising a seventh multiplexed-inhibitor bead carrying immobilized VI16832.

The present disclosure also provides multi-analyte columns comprising a first layer comprising a first multiplexed-inhibitor bead carrying immobilized SB203580; a second layer comprising a second multiplexed-inhibitor bead carrying immobilized lapatinib; a third layer comprising a third multiplexed-inhibitor bead carrying immobilized dasatinib; a fourth layer comprising a fourth multiplexed-inhibitor bead carrying immobilized purvalanol dasatinib; a fifth layer comprising a fifth multiplexed-inhibitor bead carrying immobilized purvalanol B; a sixth layer comprising a sixth multiplexed-inhibitor bead carrying immobilized purvalanol VI16832; and a seventh layer comprising a seventh multiplexed-inhibitor bead carrying immobilized purvalanol PP58. In some embodiments, the first, second, third, fourth, fifth, sixth, and seventh layers are arranged sequentially top to bottom in the order first layer (top)-second layer-third layer-fourth layer-fifth layer-sixth layer-seventh layer (bottom). In some embodiments, the first, second, third, fourth, fifth, sixth, and seventh layers are arranged sequentially top to bottom in the order seventh layer (top)-sixth layer-fifth layer-fourth layer-third layer-second layer-first layer (bottom). In some embodiments, the layers can be arranged in any order.

The present disclosure also provides methods of generating a kinome profile of a cell comprising: enriching protein kinases from a cell-containing sample; detecting the enriched kinases; and transforming the data obtained from the detected and/or quantified enriched kinases into the kinome profile.

In some embodiments, the kinase enrichment step comprises applying a cell lysate to a multi-analyte column; washing the multi-analyte column to remove any unbound proteins; and eluting kinases bound to the multi-analyte column with a denaturing agent (e.g., urea and guanidine-HCl. In some embodiments, the kinase content is further enriched via application of the eluate to a second multi-analyte column having either the same or different composition.

Methods of producing cell lysates are known in the art and include, without limitation, physical methods such as mechanical lysis (for example using a Waring blender), liquid homogenization, sonication or manual lysis (for example using a pestle and mortar), methanol/chloroform protein precipitation, or detergent-based methods such as CHAPS or Triton-X. Typically, the cells are lysed using a denaturing buffer such as a urea-based buffer. Lysate from any cell type can be used to load the multi-analyte column according to the present disclosure. Suitable primary cells can be directly obtained from tissue or body fluids, or human tissue or body fluids, and can be, for example, blood cells, lymphocytes, neuronal cells, glial cells, epithelial cells, fibroblastic cells, hepatocytes, muscle cells or cardiomyocytes. In some embodiments, the cell is a healthy cell. In some embodiments, the cell is a diseased cell (e.g., cancer cell).

Methods of detection of kinases are well known in the art. In some embodiments, the detection may be carried out by mass spectroscopy (MS). Methods of mass spectrometry analysis are well known to those skilled in the art (see, for example, Yates, J. Mass Spect., 1998, 33, 1-19; Kinter and Sherman, Protein Sequencing and Identification Using Tandem Mass Spectrometry, John Wiley & Sons, New York (2000); Aebersold and Goodlett, Chem. Rev., 2001, 101, 269-295; Griffin et al., Curr. Opin. Biotechnol., 2001, 12, 607-612). A variety of mass spectrometry systems can be employed in the methods described herein for identifying and/or quantifying a biomolecule in a sample, such as a polypeptide. Mass analyzers with high mass accuracy, high sensitivity and high resolution include, but are not limited to, matrix-assisted laser desorption time-of-flight (MALDI-TOF) mass spectrometers, electrospray ionization time-of-flight (ESI-TOF) mass spectrometers, Fourier transform ion cyclotron mass analyzers (FT-ICR-MS), and ORBITRAP™ analyzer instruments. Other modes of MS include ion trap and triple quadrupole mass spectrometers. In ion trap MS, analytes are ionized by electrospray ionization or MALDI and then put into an ion trap. Trapped ions can then be separately analyzed by MS upon selective release from the ion trap. Ion traps can also be combined with the other types of mass spectrometers described herein. Fragments can also be generated and analyzed. For high resolution peptide fragment separation, liquid chromatography ESI-MS/MS or automated LC-MS/MS, which utilizes capillary reverse phase chromatography as the separation method, can be used (Yates et al., Methods Mol. Biol., 1999, 112, 553-569). Data dependent collision-induced dissociation (CID) with dynamic exclusion can also be used as the mass spectrometric method (Goodlett et al., Anal. Chem., 2000, 72, 1112-1118). In some embodiments, detection is carried out by liquid chromatography-mass spectrometry (LC-MS). Optionally, modified peptides (typically phosphorylated peptides) in the peptide mixture following trypsin digestion step of MS sample preparation are enriched using a IMAC or TiO₂ chromatography (Thingholm et al., 2006, Nat. Prot., 1, 1929-1935).

The present disclosure also provides for quantification of enriched kinases through comparing the levels of kinases present in a sample to the levels of kinases in a standard mixture. As used herein, the term “standard mixture” refers to a mixture of one or a plurality of reference kinases having been obtained via extraction from at least two different cell populations, and wherein the one or plurality of reference kinases are a labeled form of the one or the plurality of kinases (as defined herein) in the sample. The term “labeled form” as used herein includes an isotope labeled form. In some embodiments, the labeled form is a chemically or metabolically labeled isotope. In some embodiments, the labeled form is a metabolically labeled isotope form of the biomolecule.

Suitable “isotope labeled forms” of biomolecules include variants of naturally occurring molecules, in whose structure one or more atoms have been substituted with atom(s) of the same element having a different atomic weight, although isotope labeled forms in which the isotope has been covalently linked either directly or via a linker, or wherein the isotope has been complexed to the biomolecule are likewise contemplated. In either case, the isotope can be a stable isotope.

A “stable isotope”, as referred to herein, is a non-radioactive isotopic form of an element having identical numbers of protons and electrons, but having one or more additional neutron(s), which increase(s) the molecular weight of the element. In some embodiments, the stable isotope is selected from the group consisting of ²H, ¹³C, ¹⁵N, ¹⁷O, ¹⁸O, ³³P, and ³⁴S, or any combination thereof. In some embodiments, the stable isotope is ¹³C and/or ¹⁵N.

The labeling can be affected by means known in the art. A labeled reference biomolecule can be synthesized using isotope labeled amino acids or peptides as precursor molecules. For example, isotope-coded affinity tag (ICAT) reagents label reference biomolecules such as proteins at the alkylation step of sample preparation (Gygi et al., Nat. Biotechnol., 1999, 17, 994-999, PCT Publication WO 00/11208). Visible ICAT reagents (VICAT reagents) may be likewise employed (PCT Publication WO 04/019000), whereby the VICAT-type reagent contains as a detectable moiety a fluorophore or radiolabel. iTRAQ and similar methods may likewise be employed (Ross et al., Mol. Cell. Proteomics, 2004, 3, 1154-1169). Metabolic labeling can also be used to produce the labeled reference biomolecules. For example, cells can be grown on media containing isotope labeled precursor molecules, such as isotope labeled amino acids, which are incorporated into proteins or peptides, which are thereby metabolically labeled. In some embodiments, the metabolic isotope labeling is a stable isotope labeling with amino acids in cell culture (SILAC); (Olsen et al., Cell, 2006, 127, 635-648). If metabolic labeling is used, and the labeled form of the one or the plurality of reference biomolecules is a SILAC labeled form of the reference biomolecule/s, the standard mixture as defined herein is also referred to as SUPER-SILAC mix.

Additional methods of labeling are also well known in the art and include chemical derivatization (e.g., iTRAQ (Ross et al., Mol. Cell. Proteomics, 2004, 3, 1154-69), ICAT (Gygi et al., Nat. Biotechnol., 1999, 17, 994-999), and TMT (Dayon et al., Anal. Chem., 2008, 80, 2921-31) techniques.

The present disclosure also provides cancer type-independent standard kinase mixtures comprising equal amounts of kinases extracted from at least two cancer cell lines and labeled according to the methods described herein. In some embodiments, the standard kinase mixture comprises kinases extracted from two cancer cell lines and labeled according to the methods described herein. In some embodiments, the standard kinase mixture comprises equal amounts of kinases extracted from three cancer cell lines and labeled according to the methods described herein. In some embodiments, the standard kinase mixture comprises equal amounts of kinases extracted from three cancer cell lines and labeled according to the methods described herein. In some embodiments, the standard kinase mixture comprises equal amounts of kinases extracted from four cancer cell lines and labeled according to the methods described herein. In some embodiments, the standard kinase mixture comprises equal amounts of kinases extracted from five cancer cell lines and labeled according to the methods described herein. In some embodiments, the standard kinase mixture comprises equal amounts of kinases extracted from more than five cancer cell lines and labeled according to the methods described herein. In some embodiments, standard kinase mixture comprises equal amounts of kinases extracted from UACC257, MOLT4, COL0205, ACHN and PC3 cancer cell lines (herein termed “Heavy Kinome Standard” (HKS)).

The present disclosure also provides for other types of standard kinase mixtures, for example a kinome standard specific for a particular cancer, for example a mixture of kinases extracted from a set of cell lines originating from a particular cancer, or obtained from a set patient diagnosed with a particular cancer.

The present disclosure also provides quantification of kinase levels in both the standard mixture and a patient sample. In some embodiments, when the standard kinase mixture contains kinases labeled according to methods describe herein, the quantification of standard and sample kinase levels may be carried out in parallel, for example by assaying a 1:1 mixture of a patient sample and the standard kinase mixture.

In some embodiments, the MS data can be acquired using data dependent acquisition method (DDA) with an inclusion list targeting high frequency peptides to ensure quantitation of common kinase peptides across samples. In some embodiments, the MS data can be acquired using data independent acquisition method (DIA; Li et al., Nat. Meth., 2015, 12, 1105-1106), targeting peptides from low frequent kinases (i.e., those detected ≤10% in 50 tumor kinome profiling experiments). In some embodiments, the data is acquired using a combination of DDA and DIA, wherein each method is applied to half of the analyzed sample.

Mass-spectrometric analysis of the biomolecules in a sample, e.g., a tissue, with the standard mixture, can be targeted, and can include SIM-scans (single ion monitoring) to increase sensitivity, e.g., 10- to 100-fold or more. It is possible to pre-determine one or a plurality of biomolecules of interest, such as peptides of interest, and target the analysis to them (see e.g., U.S. Pat. No. 9,905,405). Novel algorithms can match between a “master run” and the experiment run in real time, and identify the targeted biomolecule(s). When the biomolecule(s) is or are found, SIM scans can be performed. In the SIM-scan only the specific mass window is selectively accumulated, therefore dramatically increasing the sensitivity. It will be appreciated that the targeted biomolecule can be detected only in the standard mixture according to the methods described herein, and then, with the increased sensitivity, it will be monitored also in the sample. With the SIM scan, it is then possible to achieve accurate quantification even for biomolecules that are very low in the sample. Furthermore, in depth characterization of the standard mixture before the quantitation of the biomolecules in the sample allows targeting of one or a plurality of biomolecules, e.g., the entire complement of biomolecules, for example peptides, in the sample even without their detection in the mass spectra. This is possible because the chromatographic elution time of the biomolecules of interest, such as peptides of interest, have been determined before. Furthermore, the chromatographic elution time of the other biomolecules in the standard mixture can be used to precisely estimate the elution time of the biomolecule(s) of interest during the measurements.

It will be appreciated by those skilled in the art, that other mass-spectrometry quantification methods can be particularly useful. These include, without limitation, targeted mass spectrometry methods such as Single Reaction Monitoring (SRM) or Multiple Reaction Monitoring (MRM) techniques known in the art. These methods can be used to increase the sensitivity of the measurement because instead of monitoring a narrow mass range, specific transitions from the precursor to specific fragments are monitored. In some embodiments, multiple low intensity analytes can be quantified in parallel, using the parallel reaction monitoring (PRM), where separated precursors have their populations mixed together again and then acquired in a single high resolution spectrum. In some embodiments, sequential window acquisition of all theoretical mass spectra (SWATH) method can be used.

The data relating to the peptides in the sample (typically in the form of an MS datafile and more typically an LC-MS datafile) is compared with data in a database of peptides using a computer program. For example, the mass to charge (m/z) ratio, charge (z) and relative retention time of the peptides in the sample are compared with the mass to charge (m/z) ratio, charge (z) and relative retention time of the peptides in the database. This enables the identification and quantification of each peptide in the sample using the database of peptides.

For example, the computer program is the program termed PESCAL (Cutillas et al., Mol. Cell. Proteomics, 2007, 6, 1560-73). PESCAL constructs extracted ion chromatograms (XIC, i.e., an elution profile) for each of the modified peptides (typically phosphorylated peptides) present in the database across all the samples that are to be compared. This is done by centering the XIC on the m/z and retention time of the peptide previously identified to be modified (typically phosphorylated) (i.e., present in the database constructed in the first step of the procedure). PESCAL also considers the charge of the peptide to help in the correct assignment of identity. The program also calculates the peak height and area under the curve of each XIC. The data is normalized by dividing the intensity reading (peak areas or heights) of each modified peptide (typically phosphorylated peptide) that is being analyzed by those of the reference modified peptides (typically reference phosphorylated peptides).

The present disclosure also provides methods of diagnosing cancer in a subject comprising: obtaining a kinome profile from the subject; and comparing the kinome profile to a standard cancer kinome profile; wherein the presence of the measured level or phosphorylation status of at least one kinase that is comparable to the cancer standard level indicates that the subject has cancer or is at risk of developing cancer.

As used herein, the term “cancer” refers to the physiological condition in mammals that is typically characterized by unregulated cell growth. Examples of cancer include, but are not limited to, glioma, carcinoma, lymphoma, blastoma, sarcoma, and leukemia or lymphoid malignancies. More particular, examples of such cancers include leukemia (e.g., acute myelogenous leukemia (“AML”)), squamous cell cancer (e.g., epithelial squamous cell cancer), lung cancer including small-cell lung cancer, non-small cell lung cancer (“NSCLC”), adenocarcinoma of the lung and squamous carcinoma of the lung, cancer of the peritoneum, gastric or stomach cancer including gastrointestinal cancer, pancreatic cancer, glioblastoma (e.g., glioblastoma multiforme (“GBM”)), cervical cancer, ovarian cancer, liver cancer, bladder cancer, breast cancer, colon cancer, rectal cancer, colorectal cancer (e.g. colorectal carcinoma (“CRC”)), endometrial or uterine carcinoma, salivary gland carcinoma, kidney or renal cancer, prostate cancer, vulval cancer, thyroid cancer, anal carcinoma, penile carcinoma, as well as head and neck cancer. In some embodiments the cancer is a High Grade Serous Ovarian Carcinoma (HGSOC).

The subject kinome profile is compared with the HKS through measuring the relative levels of kinases in the sample and in the standard. In some embodiments, the relative levels of each kinase are determined through counting the number of peptides corresponding to each particular kinase in the patient sample and in the HKS. In some embodiments, the mean abundance of all of the peptides corresponding to a particular kinase in the patient sample is compared to the mean abundance of all of the peptides corresponding to that particular kinase in HKS. This is typically carried out by calculating the mean (arithmetic average) abundance of all the peptides corresponding to a particular kinase from the patient sample and the mean abundance of all of the peptides corresponding to a particular kinase in the standard; and dividing the mean abundance of all of the peptides corresponding to a particular kinase from the patient sample by the mean abundance of all of the peptides corresponding to a particular kinase in the HKS. The resultant figure can optionally be log₂ transformed. This method, therefore, involves comparing the means (arithmetic average) of the intensities of all the peptides corresponding to a particular kinase in the group from patient sample relative to intensities of all the peptides corresponding to a particular kinase in the HKS.

In some embodiments, the relative levels of phosphorylated kinases, rather than their absolute levels are analyzed. In some embodiments the phosphorylated kinases are specifically enriched as described herein, and the relative phosphorylation state of each kinase is determined through counting the number of phosphorylated peptides corresponding to each particular kinase in the patient sample and in the HKS. In some embodiments, the mean abundance of all of the phosphorylated peptides corresponding to a particular kinase in the patient sample is compared to the mean abundance of all of the phosphorylated peptides corresponding to a particular kinase in HKS.

The present disclosure also provides methods whereby the presence of any kinase in a patient sample at the level comparable to those in the HKS indicates that the subject has cancer or is at risk of developing cancer. As used herein, the term “comparable” refers to the sample and standard values that are either equal or have a ratio of 0.8 to 1.2, or the values that are within no more than 20% of each other, or the value of log₂ of the ratio of the patient sample and the HKS mean abundances of all of the peptides corresponding to a particular kinase ranges −0.32 to 0.27.

Thus, in some embodiments, when the ratio of levels of a kinase in a patient sample and HKS equals 0.8, the levels in two samples are comparable. In some embodiments, when the ratio of levels of a kinase in a patient sample and HKS equals 0.85, the levels in two samples are comparable. In some embodiments, when the ratio of levels of a kinase in a patient sample and HKS equals 0.9, the levels in two samples are comparable. In some embodiments, when the ratio of levels of a kinase in a patient sample and HKS equals 0.95, the levels in two samples are comparable. In some embodiments, when the ratio of levels of a kinase in a patient sample and HKS equals 1, the levels in two samples are comparable. In some embodiments, when the ratio of levels of a kinase in a patient sample and HKS equals 1.05, the levels in two samples are comparable. In some embodiments, when the ratio of levels of a kinase in a patient sample and HKS equals 1.1, the levels in two samples are comparable. In some embodiments, when the ratio of levels of a kinase in a patient sample and HKS equals 1.15, the levels in two samples are comparable. In some embodiments, when the ratio of levels of a kinase in a patient sample and HKS equals 1.2, the levels in two samples are comparable.

In some embodiments, when the levels of a kinase in a patient sample and HKS are within 20% of each other, the levels in two samples are comparable. In some embodiments, when the levels of a kinase in a patient sample and HKS are within 19% of each other, the levels in two samples are comparable. In some embodiments, when the levels of a kinase in a patient sample and HKS are within 18% of each other, the levels in two samples are comparable. In some embodiments, when the levels of a kinase in a patient sample and HKS are within 17% of each other, the levels in two samples are comparable. In some embodiments, when the levels of a kinase in a patient sample and HKS are within 16% of each other, the levels in two samples are comparable. In some embodiments, when the levels of a kinase in a patient sample and HKS are within 15% of each other, the levels in two samples are comparable. In some embodiments, when the levels of a kinase in a patient sample and HKS are within 14% of each other, the levels in two samples are comparable. In some embodiments, when the levels of a kinase in a patient sample and HKS are within 13% of each other, the levels in two samples are comparable. In some embodiments, when the levels of a kinase in a patient sample and HKS are within 12% of each other, the levels in two samples are comparable. In some embodiments, when the levels of a kinase in a patient sample and HKS are within 11% of each other, the levels in two samples are comparable. In some embodiments, when the levels of a kinase in a patient sample and HKS are within 10% of each other, the levels in two samples are comparable. In some embodiments, when the levels of a kinase in a patient sample and HKS are within 9% of each other, the levels in two samples are comparable. In some embodiments, when the levels of a kinase in a patient sample and HKS are within 8% of each other, the levels in two samples are comparable. In some embodiments, when the levels of a kinase in a patient sample and HKS are within 13% of each other, the levels in two samples are comparable. In some embodiments, when the levels of a kinase in a patient sample and HKS are within 7% of each other, the levels in two samples are comparable. In some embodiments, when the levels of a kinase in a patient sample and HKS are within 6% of each other, the levels in two samples are comparable. In some embodiments, when the levels of a kinase in a patient sample and HKS are within 5% of each other, the levels in two samples are comparable. In some embodiments, when the levels of a kinase in a patient sample and HKS are within 4% of each other, the levels in two samples are comparable. In some embodiments, when the levels of a kinase in a patient sample and HKS are within 3% of each other, the levels in two samples are comparable. In some embodiments, when the levels of a kinase in a patient sample and HKS are within 2% of each other, the levels in two samples are comparable. In some embodiments, when the levels of a kinase in a patient sample and HKS are within 1% of each other, the levels in two samples are comparable.

In some embodiments, when log₂ of the ratio of the patient sample and the HKS mean abundances of all of the peptides corresponding to a particular kinase is −0.32, the levels in two samples are comparable. In some embodiments, when log₂ of the ratio of the patient sample and the HKS mean abundances of all of the peptides corresponding to a particular kinase is −0.3, the levels in two samples are comparable. In some embodiments, when log₂ of the ratio of the patient sample and the HKS mean abundances of all of the peptides corresponding to a particular kinase is −0.27, the levels in two samples are comparable. In some embodiments, when log₂ of the ratio of the patient sample and the HKS mean abundances of all of the peptides corresponding to a particular kinase is −0.25, the levels in two samples are comparable. In some embodiments, when log₂ of the ratio of the patient sample and the HKS mean abundances of all of the peptides corresponding to a particular kinase is −0.23, the levels in two samples are comparable. In some embodiments, when log₂ of the ratio of the patient sample and the HKS mean abundances of all of the peptides corresponding to a particular kinase is −0.2, the levels in two samples are comparable. In some embodiments, when log₂ of the ratio of the patient sample and the HKS mean abundances of all of the peptides corresponding to a particular kinase is −0.17, the levels in two samples are comparable. In some embodiments, when log₂ of the ratio of the patient sample and the HKS mean abundances of all of the peptides corresponding to a particular kinase is −0.15, the levels in two samples are comparable. In some embodiments, when log₂ of the ratio of the patient sample and the HKS mean abundances of all of the peptides corresponding to a particular kinase is −0.13, the levels in two samples are comparable. In some embodiments, when log₂ of the ratio of the patient sample and the HKS mean abundances of all of the peptides corresponding to a particular kinase is −0.1, the levels in two samples are comparable. In some embodiments, when log₂ of the ratio of the patient sample and the HKS mean abundances of all of the peptides corresponding to a particular kinase is −0.07, the levels in two samples are comparable. In some embodiments, when log₂ of the ratio of the patient sample and the HKS mean abundances of all of the peptides corresponding to a particular kinase is −0.05, the levels in two samples are comparable. In some embodiments, when log₂ of the ratio of the patient sample and the HKS mean abundances of all of the peptides corresponding to a particular kinase is −0.03, the levels in two samples are comparable. In some embodiments, when log₂ of the ratio of the patient sample and the HKS mean abundances of all of the peptides corresponding to a particular kinase is 0, the levels in two samples are comparable. In some embodiments, when log₂ of the ratio of the patient sample and the HKS mean abundances of all of the peptides corresponding to a particular kinase is 0.03, the levels in two samples are comparable. In some embodiments, when log₂ of the ratio of the patient sample and the HKS mean abundances of all of the peptides corresponding to a particular kinase is 0.05, the levels in two samples are comparable. In some embodiments, when log₂ of the ratio of the patient sample and the HKS mean abundances of all of the peptides corresponding to a particular kinase is 0.07, the levels in two samples are comparable. In some embodiments, when log₂ of the ratio of the patient sample and the HKS mean abundances of all of the peptides corresponding to a particular kinase is 0.1, the levels in two samples are comparable. In some embodiments, when log₂ of the ratio of the patient sample and the HKS mean abundances of all of the peptides corresponding to a particular kinase is 0.13, the levels in two samples are comparable. In some embodiments, when log₂ of the ratio of the patient sample and the HKS mean abundances of all of the peptides corresponding to a particular kinase is 0.15, the levels in two samples are comparable. In some embodiments, when log₂ of the ratio of the patient sample and the HKS mean abundances of all of the peptides corresponding to a particular kinase is 0.17, the levels in two samples are comparable. In some embodiments, when log₂ of the ratio of the patient sample and the HKS mean abundances of all of the peptides corresponding to a particular kinase is 0.2, the levels in two samples are comparable. In some embodiments, when log₂ of the ratio of the patient sample and the HKS mean abundances of all of the peptides corresponding to a particular kinase is 0.23, the levels in two samples are comparable. In some embodiments, when log₂ of the ratio of the patient sample and the HKS mean abundances of all of the peptides corresponding to a particular kinase is 0.25, the levels in two samples are comparable. In some embodiments, when log₂ of the ratio of the patient sample and the HKS mean abundances of all of the peptides corresponding to a particular kinase is 0.27, the levels in two samples are comparable.

The present disclosure also provides methods of treating cancer in a subject comprising: obtaining a cancer cell-containing sample from the subject; generating a kinome profile; comparing the kinome profile to a standard cancer kinome profile; and administering to the subject an effective amount of one or more appropriate kinase inhibitors, chemotherapeutic agents, epigenetic therapy agents, and/or additional biologically active compounds, or any combination thereof.

As used herein, the term “treatment”, “treat” or “treating” refers to any act intended to ameliorate the health status of patients such as therapy, prevention, prophylaxis and retardation of the disease. In some embodiments, such term refers to the amelioration or eradication of a disease or symptoms associated with a disease. In some embodiments, this term refers to minimizing the spread or worsening of the disease resulting from the administration of one or more therapeutic agents to a subject with such a disease. In particular, the term “to treat a cancer”, “treating a cancer”, “to treat a tumor” or “treating a tumor” means reversing, alleviating, inhibiting the progress of, or preventing, either partially or completely, the growth of tumors, tumor metastases, or other cancer-causing or neoplastic cells in a patient trough eradication of tumor cells, inhibiting of tumor cell growth, reducing tumor cell growth, induction of tumor cell apoptosis, suppressing tumor resistance to other therapies or any combination thereof.

Any of the numerous examples of kinase inhibitors known in the art and described herein can be used in the methods according to the present disclosure. In some embodiments a kinase inhibitor specific to a kinase identified as elevated in the kinome analysis can be administered to inhibit the function of an elevated kinase. In some embodiments an inhibitor that inhibits multiple kinases is used. In some embodiments, a combination of kinase inhibitors is used.

In some embodiments, the cancer treated using the methods described herein is High Grade Serous Ovarian Carcinoma (HGSOC), and the one or more protein kinases targeted by the kinase inhibitors administered to the subject are selected from the group of BRAF, MEK, ERK, FAK1, P70S6, AURKA, WEE1, CHEK1, CDK7, EphA, ATR, PI3KM/MTOR, CDK4, CDK6, IGF1R, EGFR-HER2, PAN-TK, NUAK, EIF2AK2, STK38, MRCKA, PAK4, PRPKCQ, AAK, CDK1, JAK1, ERBB4, DDR1, FGFR2, AKT2, PTK2B, MAPK1, and MAPK14. In some embodiments, the kinase inhibitors administered to the subject inhibit at least two of the kinases listed herein. In some embodiments, kinase inhibitors administered to the subject having HGSOC inhibit PAK4 (e.g., PF-3758309) and at least one other kinase. In some embodiments, the kinase inhibitors administered to the subject having HGSOC inhibit one or more of MRCKA DDR1, FGFR2, ERBB4, STK38, AAK1, NUAK, FAK1, and PLK3. In some embodiments, the kinase inhibitors administered to the subject having HGSOC inhibit MRCKA and at least one other kinase. In some embodiments, kinase inhibitors administered to the subject inhibit MRCKA and at least one of AAK1, STK38, PLK3, CDK7, BRD4, WEE1, FAK1, and CHEK1, or any combination thereof. MRSKA inhibitors suitable for HGSOC treatment include, without limitation, BDP5290, RKI-1447, SP772077, GSK46317A, A647563, or a combination thereof, whereas the AAK1, STK38, PLK3, CDK7, BRD4, WEE1, FAK1, and CHEK1 inhibitors suitable for HGSOC treatment in combination with one or more MRSKA inhibitors include, without limitation, GDL-0941, THZ1, JQ1, MK1775, AZD7762, and PND-1186, or any combination thereof. Thus, in some embodiments, the present disclosure provides therapies comprising a combination of at least two groups of kinase inhibitors, wherein the first group comprises one or more of BDP5290, RKI-1447, SP772077, GSK46317A, and A647563 and the second group comprises one or more of GDL-0941, THZ1, JQ1, MK1775, AZD7762, and PND-1186. In some embodiments, the kinase inhibitors administered to the subject having HGSOC inhibit STK38 and PLK3 kinases. In some embodiments, PLK3 inhibitor suitable for HGSOC treatment is GDC-0941. In some embodiments, the kinase inhibitors administered to the subject having HGSOC inhibit STK38 and CDK7 kinases. In some embodiments, CDK7 inhibitor suitable for HGSOC treatment is THZ1. In some embodiments the kinase inhibitors administered to the subject having HGSOC inhibit AAK1 kinase (e.g., A647563).

As used herein, the term “administer” refers to the placement of a composition into a subject by a method or route which results in at least partial localization of the composition at a desired site such that desired effect is produced. Administration can be by any appropriate route known in the art including, but not limited to, oral or parenteral routes, including intravenous, intramuscular, subcutaneous, transdermal, airway (aerosol), pulmonary, nasal, rectal, and topical (including buccal and sublingual) administration. Exemplary modes of administration include, but are not limited to, injection, infusion, instillation, inhalation, or ingestion. “Injection” includes, without limitation, intravenous, intramuscular, intraarterial, intrathecal, intraventricular, intracapsular, intraorbital, intracardiac, intradermal, intraperitoneal, transtracheal, subcutaneous, subcuticular, intraarticular, sub capsular, subarachnoid, intraspinal, intracerebro spinal, and intrasternal injection and infusion. In some embodiments, the compositions are administered by intravenous infusion or injection.

As used herein, the term “effective amount” means that amount of a compound, material, or composition comprising a compound described herein which is effective for producing some desired therapeutic effect in at least a sub-population of cells in a subject at a reasonable benefit/risk ratio applicable to any medical treatment. Thus, “effective amount” means that amount which, when administered to a subject for treating a disease, is sufficient to effect such treatment for the disease.

Determination of an effective amount is well within the capability of those skilled in the art. Generally, the actual effective amount can vary with the specific compound, the use or application technique, the desired effect, the duration of the effect and side effects, the subject's history, age, condition, sex, as well as the severity and type of the medical condition in the subject, and administration of other pharmaceutically active agents. Accordingly, an effective dose of compound or composition is an amount sufficient to produce at least some desired therapeutic effect in a subject.

The data obtained in vitro and in animal studies can be used in formulating a range of dosage for use in humans. The dosage of such compounds can be within a range of circulating concentrations that include the IC₅₀ with little or no toxicity. The dosage may vary within this range depending upon the dosage form employed and the route of use or administration utilized.

The effective dose can be estimated initially from the in vitro assays. A dose can be formulated in animal models to achieve a circulating plasma concentration range that includes the IC₅₀ (i.e., the concentration of the therapeutic which achieves a half-maximal inhibition of symptoms) as determined in cell culture. The effect of any particular dosage can be monitored by a suitable bioassay.

The present disclosure also provides methods of assessing a cancer therapy regimen comprising: obtaining a first cancer cell-containing sample from a subject; generating a first kinome profile; treating the subject with one or more kinase inhibitors, chemotherapy agents, or epigenetic therapies; obtaining a second cancer cell-containing sample from the subject; generating a second kinome profile; comparing the first kinome profile and the second kinome profile; and modifying the treatment regimen based on the changes in the second kinome profile as compared to the first kinome profile. In some embodiments, the efficacy of a cancer therapy assessed through comparing levels or phosphorylation states of cancer-associated kinases observed prior to treatment to those observed following treatment. In some embodiments, the a single post-treatment sample is collected and evaluated. In some embodiments, multiple samples are collected and evaluated in the course of treatment. After having monitored kinase levels or phosphorylation state for a period of time during treatment, an assessment can be made as to one or more of the following: (i) the patient is progressing well on the treatment, (ii) the treatment is effective; (iii) the patient is favorably responding to the treatment; and/or (iv) the patient's cancer is not advancing, or has been ameliorated or eliminated by the treatment. Thus, for example, a decrease in the levels or phosphorylation of a cancer-associated kinase in the post-therapy patient sample as compared to the pre-therapy patient sample would indicate that the administered therapy is effective. On the other hand, the levels remaining the same before and after therapy administration would indicate that the therapy had no effect.

In some embodiments, the methods assessing a cancer therapy regimen comprise comparing the levels or phosphorylation of a cancer-associated kinase in the post-therapy patient with HKS (for example when the pre-therapy patient sample is not available). Thus, for example, the levels or phosphorylation of a cancer-associated kinase in the post-therapy patient sample being outside of comparable range with HTS levels would indicate that the administered therapy is effective. On the other hand, the levels or phosphorylation of a cancer-associated kinase in the post-therapy patient sample being within comparable range with HTS levels would indicate that the administered therapy is ineffective.

In accordance with the present disclosure, such methods of monitoring and assessment of the levels or phosphorylation of a cancer-associated kinase during a patient's course of treatment or therapy, compared with the levels or phosphorylation of a cancer-associated kinase prior to therapy or to HKS, can provide the physician or clinician with a determination of a cancer patient's progress or lack thereof, as a consequence of a particular treatment or therapy. Such a determination allows tailoring of the cancer or anti-neoplastic treatment or therapy to better or more aggressively attack (or treat) a cancer and to select the most appropriate treatment, or course of treatment or therapy for an individual patient. This approach also allows the practitioner to determine whether dosage or mode of administration should be altered, or whether the drug regimen should be modified, for example, by combining therapies or discontinuing therapies, to try to achieve a more effective overall treatment and outcome for the patient. As an example, if it is determined by way of practicing the present disclosure that therapy is ineffective, the patient can be treated more rigorously, such as by using systemic chemotherapy and/or radiation therapy, or other treatment combinations. Conversely, when that therapy is found effective, less aggressive therapies can be decided upon. The ability to select a personalized course of therapy or treatment regimen, i.e., to be able to choose a less aggressive treatment at or close to the start of treatment, or to alter treatment from aggressive to less aggressive at a time prior to the conventional end of a treatment regimen on the basis of the monitoring analysis methods described herein, can provide less anguish and suffering for the patient on both an emotional and physical level.

The present disclosure also provides methods for predicting the development of resistance to a chemotherapy regimen in a subject, comprising: obtaining a cancer cell-containing sample from a subject; generating a kinome profile; and comparing the kinome profile to a standard cancer kinome profile; wherein the presence of the measured level or phosphorylation status of at least one kinase that is comparable to the cancer standard level indicates that the subject is at an increased risk for development of resistance to the chemotherapy regimen. As described herein, increased levels of several kinases were found to be associated with tumor resistance to various cancer therapies (e.g., carboplatin or PARP inhibitors). Thus, in some embodiments, the present disclosure provides methods of predicting the likelihood of tumor resistance and appropriately adjusting the therapeutic regimen to avoid therapies to which the tumor may prove resistant. In some embodiments, the present disclosure provides methods of overcoming tumor resistance the method comprising administering the patient a combination therapy that includes inhibiting a resistance-associated kinase. In some embodiments, the present disclosure provides methods of identification and inhibition of kinase signaling pathways activated in response to a cancer therapy.

The present disclosure also provides methods of identifying and selecting a kinase activity modulator from a group of candidate compounds comprising contacting a cell, a tissue, or an organism with a candidate compound; contacting a protein extract from the cell, the tissue, or the organism with a multi-analyte column comprising at least four layers of multiplexer inhibitor beads, wherein each layer comprises a multiplexed-inhibitor bead having at least one immobilized kinase inhibitor, and wherein the immobilized kinase inhibitor in each layer is different from the immobilized kinase inhibitor in the other layers; eluting any kinases bound to the solid supports with a denaturing agent; measuring levels of a plurality of the kinases detected to generate a kinome profile; comparing the measured kinome profile to a standard kinome profile obtained from cells that were not contacted with a compound; and using the kinome profile to select the kinase activity modulator. In some embodiments, decrease in levels or phosphorylation state of one or more kinases in response to a candidate compound treatment indicates that the compound acts as an inhibitor for these kinases. In some embodiments, the increase in levels or phosphorylation state of one or more kinases in response to a candidate compound treatment indicates that the compound acts as a stimulant for these kinases. The compounds identified according to these methods can subsequently be used in therapeutic applications, for example through targeting a specific set of kinases for inhibition or stimulation.

The present disclosure also provides uses of the kinase inhibitor therapies in combination with other clinical oncology treatments including surgery, radiation therapy, and co-administration with additional anti-cancer therapeutic agents.

Combination therapies may be useful in preventing or treating cancer and in preventing metastasis or recurrence of cancer. “Combination therapy”, as used herein, means the administration of a combination comprising at least one kinase inhibitor treatment and at least one therapeutic moiety (e.g., anti-cancer therapeutic agent) wherein the combination may have therapeutic synergy or improves the measurable therapeutic effects in the treatment of cancer over (i) the kinase inhibitor treatment used alone, or (ii) the therapeutic moiety used alone, or (iii) the use of the therapeutic moiety in combination with another therapeutic moiety without the addition of kinase inhibitor treatment. The term “therapeutic synergy”, as used herein, means the combination of an kinase inhibitor treatment and one or more therapeutic moiety(ies) having a therapeutic effect greater than the additive effect of the combination of the kinase inhibitor treatment and the one or more therapeutic moiety(ies).

Desired outcomes of the disclosed combinations are quantified by comparison to a control or baseline measurement. As used herein, relative terms such as “improve,” “increase,” or “reduce” indicate values relative to a control, such as a measurement in the same individual prior to initiation of treatment described herein, or a measurement in a control individual (or multiple control individuals) in the absence of kinase inhibitor treatments described herein but in the presence of other therapeutic moiety(ies) such as standard of care treatment. A representative control individual is an individual afflicted with the same form of cancer as the individual being treated, who is about the same age as the individual being treated (to ensure that the stages of the disease in the treated individual and the control individual are comparable.)

Changes or improvements in response to therapy are generally statistically significant. As used herein, the term “significance” or “significant” relates to a statistical analysis of the probability that there is a non-random association between two or more entities. To determine whether or not a relationship is “significant” or has “significance,” a “p-value” can be calculated. P-values that fall below a user-defined cut-off point are regarded as significant. A p-value less than or equal to 0.1, less than 0.05, less than 0.01, less than 0.005, or less than 0.001 may be regarded as significant.

A synergistic therapeutic effect may be an effect of at least about two-fold greater than the therapeutic effect elicited by a single therapeutic moiety or kinase inhibitor treatment, or the sum of the therapeutic effects elicited by the kinase inhibitor treatment or the single therapeutic moiety(ies) of a given combination, or at least about five-fold greater, or at least about ten-fold greater, or at least about twenty-fold greater, or at least about fifty-fold greater, or at least about one hundred-fold greater. A synergistic therapeutic effect may also be observed as an increase in therapeutic effect of at least 10% compared to the therapeutic effect elicited by a single therapeutic moiety or kinase inhibitor treatment or the sum of the therapeutic effects elicited by the kinase inhibitor treatment or the single therapeutic moiety(ies) of a given combination, or at least 20%, or at least 30%, or at least 40%, or at least 50%, or at least 60%, or at least 70%, or at least 80%, or at least 90%, or at least 100%, or more. A synergistic effect is also an effect that permits reduced dosing of therapeutic agents when they are used in combination.

In practicing combination therapy, the kinase inhibitor treatment and therapeutic moiety(ies) may be administered to the subject simultaneously, either in a single composition, or as two or more distinct compositions using the same or different administration routes. Alternatively, treatment with the kinase inhibitor treatment may precede or follow the therapeutic moiety treatment by, e.g., intervals ranging from minutes to weeks. In some embodiments, both the therapeutic moiety and the kinase inhibitor are administered within about 5 minutes to about two weeks of each other. In yet other embodiments, several days (2, 3, 4, 5, 6 or 7), several weeks (1, 2, 3, 4, 5, 6, 7 or 8) or several months (1, 2, 3, 4, 5, 6, 7 or 8) may lapse between administration of the kinase inhibitor and the therapeutic moiety.

The combination therapy can be administered until the condition is treated, palliated or cured on various schedules such as once, twice or three times daily, once every two days, once every three days, once weekly, once every two weeks, once every month, once every two months, once every three months, once every six months, or may be administered continuously. The kinase inhibitor and therapeutic moiety(ies) may be administered on alternate days or weeks; or a sequence of kinase inhibitor treatments may be given, followed by one or more treatments with the additional therapeutic moiety. In some embodiments, an kinase inhibitor is administered in combination with one or more therapeutic moiety(ies) for short treatment cycles. In some embodiments, the combination treatment is administered for long treatment cycles. The combination therapy can be administered via any route.

The term “anti-cancer therapeutic agent” as used herein is one subset of “therapeutic moieties”, which in turn is a subset of the agents described as “pharmaceutically active moieties”. More particularly “anti-cancer therapeutic agent” means any agent that can be used to treat a cell proliferative disorder such as cancer, and includes, but is not limited to, cytotoxic agents, cytostatic agents, anti-angiogenic agents, debulking agents, chemotherapeutic agents, radiotherapy and radiotherapeutic agents, targeted anti-cancer agents, biological response modifiers, therapeutic antibodies, cancer vaccines, cytokines, hormone therapy, anti-metastatic agents, epigenetic therapy agents, antiproliferative agents, and immunotherapeutic agents. It will be appreciated that in some embodiments as discussed herein, such anti-cancer agents may comprise antibody drug conjugates and may be associated with antibodies prior to administration.

Anti-cancer therapeutic agents can further include any chemical agent that inhibits, or is designed to inhibit, a cancerous cell or a cell likely to become cancerous or generate tumorigenic progeny (e.g., tumorigenic cells). Such chemical agents are often directed to intracellular processes necessary for cell growth or division, and are thus particularly effective against cancerous cells, which generally grow and divide rapidly. For example, vincristine depolymerizes microtubules, and thus inhibits cells from entering mitosis.

Examples of anti-cancer therapeutic agents that may be used in combination with kinase inhibitors treatment include, but are not limited to, alkylating agents, alkyl sulfonates, anastrozole, amanitins, aziridines, ethylenimines and methylamelamines, acetogenins, a camptothecin, BEZ-235, bortezomib, bryostatin, callystatin, CC-1065, ceritinib, crizotinib, cryptophycins, dolastatin, duocarmycin, eleutherobin, erlotinib, pancratistatin, a sarcodictyin, spongistatin, nitrogen mustards, antibiotics, enediyne dynemicin, bisphosphonates, esperamicin, chromoprotein enediyne antiobiotic chromophores, aclacinomysins, actinomycin, authramycin, azaserine, bleomycins, cactinomycin, canfosfamide, carabicin, carminomycin, carzinophilin, chromomycinis, cyclosphosphamide, dactinomycin, daunorubicin, detorubicin, 6-diazo-5-oxo-L-norleucine, doxorubicin, epirubicin, esorubicin, exemestane, fluorouracil, fulvestrant, gefitinib, idarubicin, lapatinib, letrozole, lonafarnib, marcellomycin, megestrol acetate, mitomycins, mycophenolic acid, nogalamycin, olivomycins, pazopanib, peplomycin, potfiromycin, puromycin, quelamycin, rapamycin, rodorubicin, sorafenib, streptonigrin, streptozocin, tamoxifen, tamoxifen citrate, temozolomide, tepodina, tipifarnib, tubercidin, ubenimex, vandetanib, vorozole, XL-147, zinostatin, zorubicin; anti-metabolites, folic acid analogues, purine analogs, androgens, anti-adrenals, folic acid replenisher such as frolinic acid, aceglatone, aldophosphamide glycoside, aminolevulinic acid, eniluracil, amsacrine, bestrabucil, bisantrene, edatraxate, defofamine, demecolcine, diaziquone, elfornithine, elliptinium acetate, epothilone, etoglucid, gallium nitrate, hydroxyurea, lentinan, lonidainine, maytansinoids, mitoguazone, mitoxantrone, mopidanmol, nitraerine, pentostatin, phenamet, pirarubicin, losoxantrone, podophyllinic acid, 2-ethylhydrazide, procarbazine, polysaccharide complex, razoxane; rhizoxin; SF-1126, sizofiran; spirogermanium; tenuazonic acid; triaziquone; 2,2′,2″-trichlorotriethylamine; trichothecenes (T-2 toxin, verracurin A, roridin A and anguidine); urethan; vindesine; dacarbazine; mannomustine; mitobronitol; mitolactol; pipobroman; gacytosine; arabinoside; cyclophosphamide; thiotepa; taxoids, chloranbucil; gemcitabine; 6-thioguanine; mercaptopurine; methotrexate; platinum analogs, vinblastine; platinum; etoposide; ifosfamide; mitoxantrone; vincristine; vinorelbine; novantrone; teniposide; edatrexate; daunomycin; aminopterin; xeloda; ibandronate; irinotecan, topoisomerase inhibitor RFS 2000; difluorometlhylornithine; retinoids; capecitabine; combretastatin; leucovorin; oxaliplatin; XL518, inhibitors of PKC-alpha, Raf, H-Ras, EGFR and VEGF-A that reduce cell proliferation and pharmaceutically acceptable salts or solvates, acids or derivatives thereof. Also included in this definition are anti-hormonal agents that act to regulate or inhibit hormone action on tumors such as anti-estrogens and selective estrogen receptor antibodies, aromatase inhibitors that inhibit the enzyme aromatase, which regulates estrogen production in the adrenal glands, and anti-androgens; as well as troxacitabine (a 1,3-dioxolane nucleoside cytosine analog); antisense oligonucleotides, ribozymes such as a VEGF expression inhibitor and a HER2 expression inhibitor; vaccines, PROLEUKIN® rIL-2; LURTOTECAN® topoisomerase 1 inhibitor; ABARELIX® rmRH; Vinorelbine and Esperamicins and pharmaceutically acceptable salts or solvates, acids or derivatives thereof.

Suitable anti-cancer agents comprise commercially or clinically available compounds such as TARCEVA® (erlotinib), TAXOTERE® (docetaxel), 5-FU (fluorouracil, 5-fluorouracil, CAS No. 51-21-8), GEMZAR® (gemcitabine), PD-0325901 (CAS No. 391210-10-9), cisplatin (cis-diamine, dichloroplatinum(II), CAS No. 15663-27-1), carboplatin (CAS No. 41575-94-4), TAXOL® (paclitaxel), HERCEPTIN® (trastuzumab), TEMODAR® and TEMODAL® (temozolomide; 4-methyl-5-oxo-2,3,4,6,8-pentazabicyclo [4.3.0] nona-2,7,9-triene-9-carboxamide, CAS No. 85622-93-1), NOLVADEX®, ISTUBAL®, and VALODEX® (tamoxifen; (Z)-2-[4-(1,2-diphenylbut-1-enyl)phenoxy]-N,N-dimethylethanamine), and ADRIAMYCIN® (doxorubicin). Additional commercially or clinically available anti-cancer agents comprise ELOXATIN® (oxaliplatin), VELCADE® (bortezomib), SUNITINIB® (sutent; SU11248), FEMARA® (letrozole), GLEEVEC® (imatinib mesylate), XL-518 (Mek inhibitor, WO 2007/044515), ARRY-886 (Mek inhibitor, AZD6244), SF-1126 (PI3K inhibitor), BEZ-235 (PI3K inhibitor), XL-147 (PI3K inhibitor), PTK787/ZK 222584, FASLODEX® (fulvestrant), leucovorin (folinic acid), RAPAMUNE® (rapamycin; sirolimus), TYKERB® (lapatinib; GSK572016), SARASAR™ (lonafarnib, SCH 66336), NEXAVAR® (sorafenib; BAY43-9006), IRESSA® (gefitinib), CAMPTOSAR® (irinotecan; CPT-11), ZARNESTRA™ (tipifarnib), ABRAXANE™ (Cremophor-free), albumin-engineered nanoparticle formulations of paclitaxel, ZACTIMA® (vandetanib; rINN, ZD6474,), chloranmbucil, AG1478, AG1571 (SU 5271), TORISEL® (temsirolimus), pazopanib, TELCYTA® (canfosfamide), CYTOXAN® (thiotepa) and NEOSAR® (cyclosphosphamide); NAVELBINE® (vinorelbine); XELODA® (capecitabine), NOLVADEX® (tamoxifen); FARESTON® (tamoxifen citrate), MEGASE® (megestrol acetate), AROMASIN® (exemestane), formestanie, fadrozole, RIVISOR® (vorozole), FEMARA® (letrozole), and ARIMIDEX® (anastrozole).

Particularly suitable anti-cancer chemotherapeutic agents of present disclosure include, without limitation, Mechlorethamine hydrochloride, Cyclophosphamide, Ifosfamide, Chlorambucil, Melphalan, Busulfan, Thiotepa (Triethylenethiophosphoramide), Carmustine, Lomustine, Streptozocin, Vincristine, Vinblastine, Paclitaxel, Methotrexate, Mercaptopurine, Thioguanine, Fluorouracil, Cytarabine, Azacitidine, Dactinomycin, Doxorubicin, Daunorubicin, Idarubicin, Bleomycin, Picamycin, Mitomycin, Hydroxyurea, Procarbazine, Dacarbazine, Cisplatin, Carboplatin, Asparaginase, Etoposide, Amsarcrine, Mitotane, Shreptozoin, Altretamine, Teniposde, Plcamydin, Fluorodeoxyuridine, CB3717, Floxuridine, Pentostatin, Cyctrabine, Fludarabine, Irinotecan, Adriamycin, Camptothecin, α-, β-, or γ-Interferon, Interleukin-2, Docetaxel, Topotecan, and Mitoxantrone, etc., or adjuvant therapies that further stimulate the immune response.

As used herein, the term “antiproliferative agent” refers to an agent having antiproliferative effects on cancer cells. Antiproliferative agents contemplated in the present disclosure include, without limitation, cisplatin, carboplatin, etoposide, tamoxifen, methotrexate, 5-fluorouracil, adriamycin, daunorubicin, doxorubicin, vincristine, and vinblastine. In some embodiments, the antiproliferative agent is selected from the group consisting of cisplatin, carboplatin, methotrexate, 5-fluorouracil, vincristine, and vinblastine. In some embodiments, the antiproliferative agent is selected from the group consisting of amsacrine, mitotane, topotecan, tretinoin, hydroxyurea, procarbazine, carmustine, mechlorethamine hydrochloride, cyclophosphamide, ifosfamide, chlorambucil, melphalan, busulfan, thiotepa, carmustine, estramustine, dacarbazine, omustine, streptozocin, vincristine, vinblastine, vinorelbine, vindesine, fludarabine, fluorodeoxyuridine, cytosine arabinoside, cytarabine, azidothymidine, cysteine arabinoside, azacytidine, mercaptopurine, thioguanine, cladribine, pentostatin, arabinosyl adenine, dactinomycin, daunorubicin, doxorubicin, amsacrine, idarubicin, mitoxantrone, bleomycin, plicamycin, ansamitomycin, mitomycin, aminoglutethimide, and flutamide.

As used herein, the term “cytotoxic agent,” which can also be an anti-cancer agent refers a substance that is toxic to the cells and decreases or inhibits the function of cells and/or causes destruction of cells. Typically, the substance is a naturally occurring molecule derived from a living organism (or a synthetically prepared natural product). Examples of cytotoxic agents include, but are not limited to, small molecule toxins or enzymatically active toxins of bacteria (e.g., Diphtheria toxin, Pseudomonas endotoxin and exotoxin, Staphylococcal enterotoxin A), fungal (e.g., α-sarcin, restrictocin), plants (e.g., abrin, ricin, modeccin, viscumin, pokeweed anti-viral protein, saporin, gelonin, momoridin, trichosanthin, barley toxin, Aleurites fordii proteins, dianthin proteins, Phytolacca americana proteins (PAPI, PAPII, and PAP-S), Momordica charantia inhibitor, curcin, crotin, Saponaria officinalis inhibitor, mitegellin, restrictocin, phenomycin, neomycin, and the tricothecenes) or animals, (e.g., cytotoxic RNases, such as extracellular pancreatic RNases; DNase I, including fragments and/or variants thereof).

As used herein, the term “epigenetic therapy agent,” as used herein refers to agents that can alter DNA modification state thereby activating and/or silencing gene expression. Examples of epigenetic therapy agents include, without limitation, histone deacetylase inhibitors, histone methyltransferase inhibitors and histone demethylases, and any combination thereof.

In some embodiments, the anti-cancer therapeutic agent administered in combination therapy with kinase inhibitors is one or more poly ADP ribose polymerase (PARP) inhibitors. PARP is an enzyme located in the nuclei of cells of various organs, including muscle, heart and brain cells. PARP plays a physiological role in the repair of strand breaks in DNA. Once activated by damaged DNA fragments, PARP catalyzes the attachment of up to 100 ADP-ribose units to a variety of nuclear proteins, including histones and PARP itself. This enzyme is thought to play a role in enhancing DNA repair. Several forms of cancer are more dependent on PARP than regular cells, making PARP an attractive target for cancer therapy. Numerous PARP inhibitors are known in the art and include, without limitation, BSI201, olaparib (AZD2281), ABT-888, AGO14699, CEP9722, MK 4827, KU-0059436 (AZD2281), LT-673,3-aminobenzamide, iniparib, talazoparib, veliparib, rucaparib, niraparib, E7016, BGB290.

In some embodiments, the anti-cancer therapeutic agent administered in combination therapy with kinase inhibitors is a bromodomain and extra-terminal domain (BET) inhibitors. Some forms of acute myeloid leukemia, multiple myeloma, and acute lymphoblastic leukemia are dependent on the BET protein BRD4, and these cancers are thus sensitive to BET inhibitors. In one of the mechanisms, expression of the growth promoting transcription factor Myc is blocked by BET inhibitors. Numerous BET inhibitors are known in the art and described, for example in U.S. Pat. No. 5,712,274, PCT Publications WO 2011/054843, WO 2012/116170, WO 2014/134583, and WO 2009/084693, and Japanese Patent Application Publication No. JP2008-156311. Examples of BET inhibitors include without limitation: JQ1, OTX015, I-BET-762, RVX-208, I-BET-762, I-BET 151 (GSK1210151A), I-BET 762 (GSK525762), I-BET-726 (GSK1324726A), TEN-010, CPI-203, CPI-0610, RVX-208, ABBV-075, BAY1238097, and LY294002.

The present disclosure also provides kits comprising one or more of the multi-analyte columns described herein. A kit may optionally further comprise a container with a predetermined amount of a purified kinase, a peptide from a kinase or a phosphopeptide from a kinase, for use as a standard or control useful in quantifying the amount of kinases in the sample. In some embodiments, the kit contains a predetermine amount of the Heavy Kinome Standard comprising equal amounts of kinases extracted from UACC257, MOLT4, COL0205, ACHN and PC3 cancer cell lines. In some embodiments, the kit comprises isotopically labeled materials such as ¹³C, ¹⁵N, or ¹⁸O. Each kit may also include printed instructions and/or a printed label describing the practicing of the methods described herein. Kit containers may optionally be sterile containers. The kits may also be configured for research use only applications whether on clinical samples, research use samples, cell lines and/or primary cells. The kits, beads, and/or columns may also be configured for uses such as drug discovery; compound validation; optimizing a therapeutic window; investigating kinome toxicity for in vitro or in vivo systems, or animal models (cell line xenographs, primary human cell xenographs in animals). The kits may also be configured for understanding a mechanism or action or basic research into the kinome in any of these systems. One of ordinary skill would readily understand a myriad of uses of the tools and methods described herein to study and improve kinome associated disorders human, animal, plant diseases, particularly cancers. The kinome has great importance learning, immunological disorders and developmental biology. With regard to plant kinomes, see exemplary studies of the rice kinome and the Arabidopsis kinome (Dardic et al., Plant Physiology, 2007, 143, 579-586; and Ritsema et al., Plant Methods, 2007 3:3 doi:10.1186/1746-4811-3-3).

Throughout the specification the word “comprising,” or variations such as “comprises” or “comprising,” will be understood to imply the inclusion of a stated element, integer or step, or group of elements, integers or steps, but not the exclusion of any other element, integer or step, or group of elements, integers or steps. The present disclosure may suitably “comprise”, “consist of”, or “consist essentially of”, the steps, elements, and/or reagents described in the claims.

In order that the subject matter disclosed herein may be more efficiently understood, examples are provided below. It should be understood that these examples are for illustrative purposes only and are not to be construed as limiting the claimed subject matter in any manner. Throughout these examples, molecular cloning reactions, and other standard recombinant DNA techniques, were carried out according to methods described in Maniatis et al., Molecular Cloning—A Laboratory Manual, 2nd ed., Cold Spring Harbor Press (1989), using commercially available reagents, except where otherwise noted.

### EXAMPLES

### Example 1: Quantifying the Kinome within and Across Tumors Using Quantitative Multiplexed Inhibitor Beads (QMIBs) and Mass Spectrometry

A mass spectrometry-based technique that enables the activity and/or levels of the human kinome to be analyzed simultaneously at the proteomic level was used, providing an unparalleled assay to explore the dark cancer kinome. Quantitative kinome profiling of HGSOC patient tumors, PDX tumors, and cell lines using Multiplexed Inhibitor beads and Mass Spectrometry (MIB/MS) revealed a kinome signature composed of previous established kinase drivers, and a large network of dark kinases with no established cancer function in HGSOC. RNAi-mediated knockdown of the dark kinases AAK1, MRCKA, STK38, NUAK1, or STK35 in HGSOC cell lines blocked cell growth and, in some instances, induced apoptosis, demonstrating the dependency of HGSOC cells for these kinases. Proteomic characterization of HGSOC cells depleted of the dark kinase MRCKA, revealed a role in FAK1, PAK4, AURKA and/or CHEK1 survival signaling in HGSOC cells. Genetic depletion or small molecule inhibition of MRCKA reduced FAK1 and MLC phosphorylation leading to defects in HGSOC cell migration. Notably, genetic depletion of MRKCA synergized with several drug therapies currently approved or in clinical trials for HGSOC, supporting MRCKA as a promising kinase target for combination therapies in HGSOC. Collectively, the proteomic evaluation of the ovarian cancer kinome identified several dark kinases that represent new therapeutic avenues for the treatment of HGSOC and highlight the kinase MRCKA as a new anti-cancer target in HGSOC.

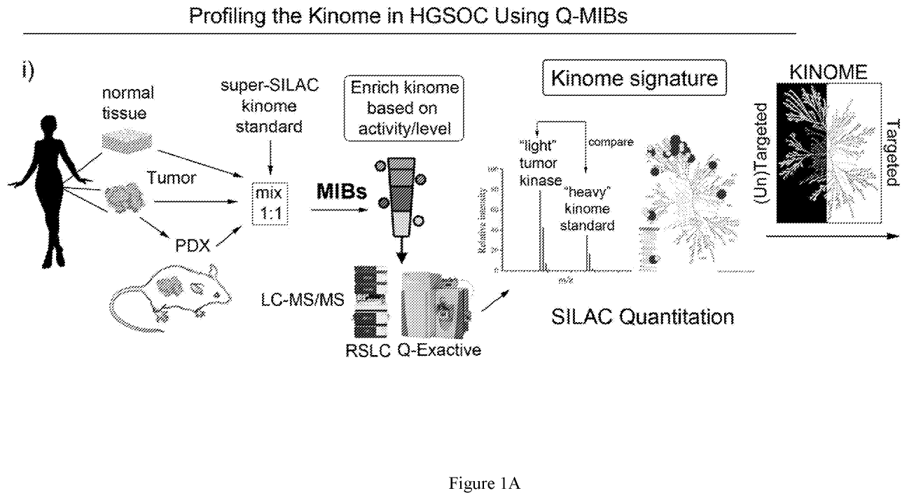

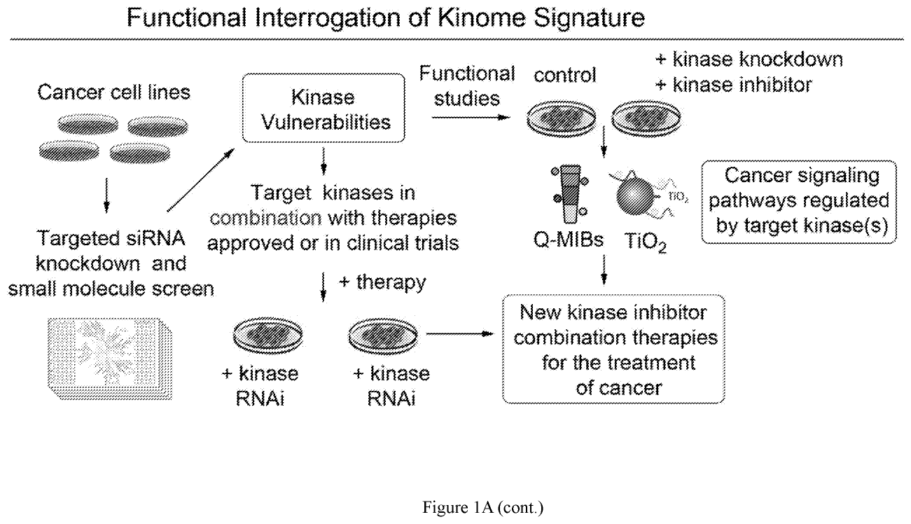

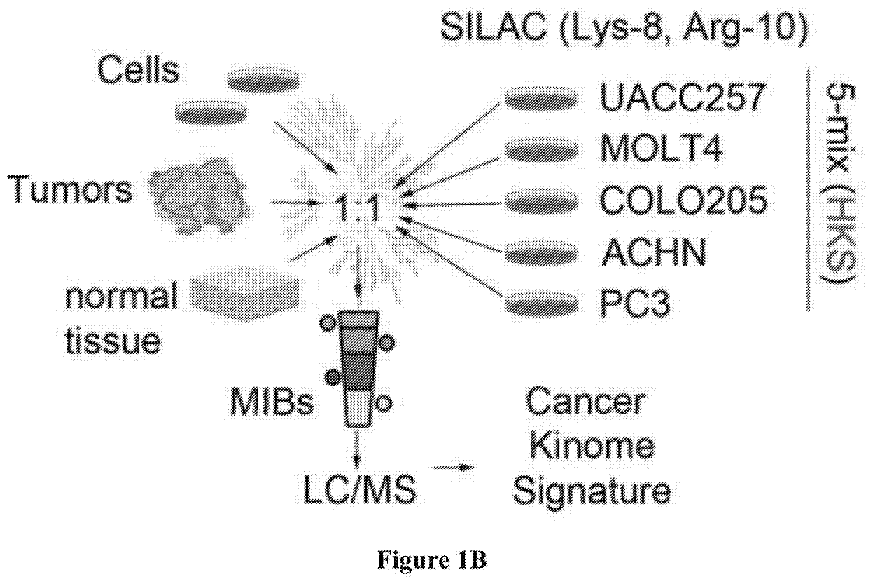

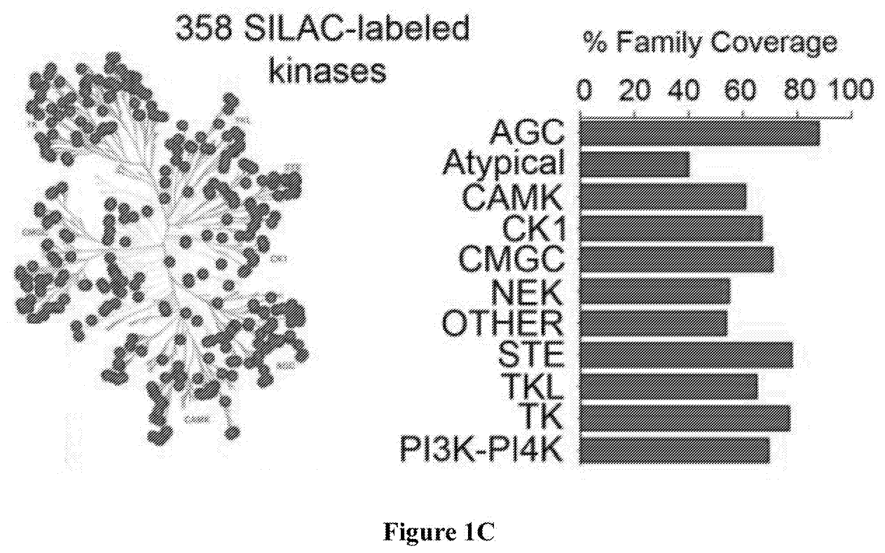

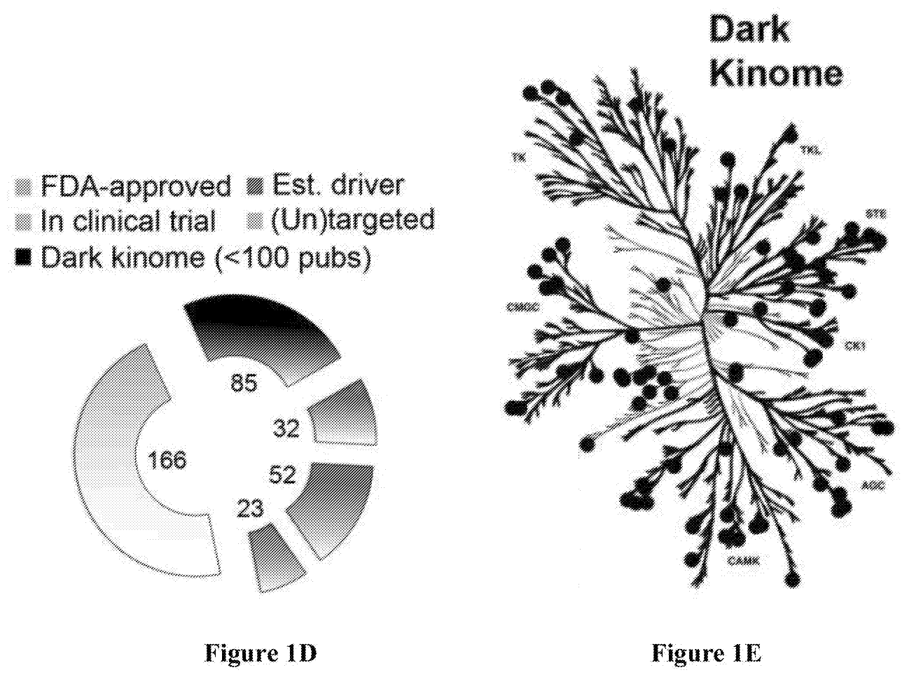

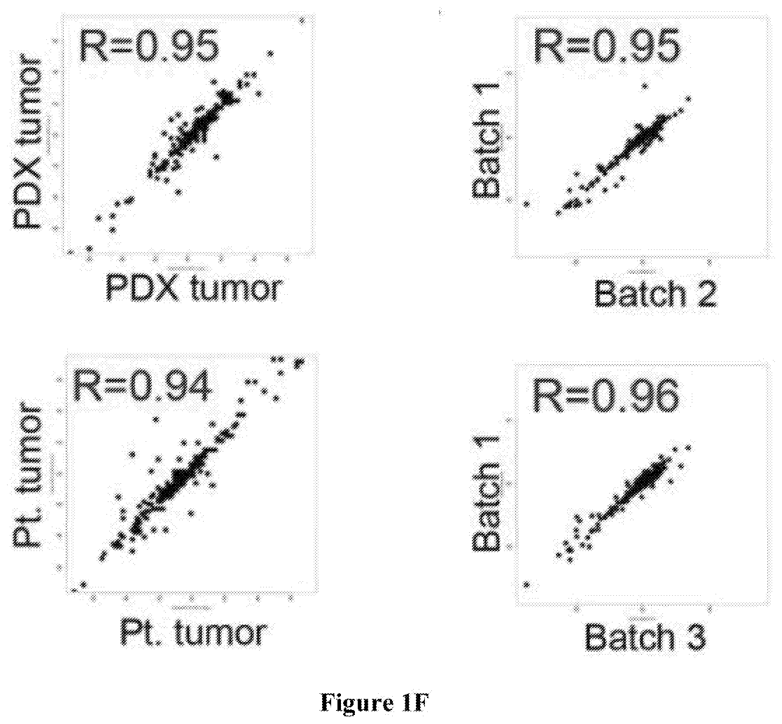

To probe the therapeutic potential of the HGSOC kinome, a chemical proteomics approach was applied that combines kinase affinity capture using MIB/MS (see, FIG. 1A). MIBs consists of a layered mixture of immobilized ATP-competitive pan-kinase inhibitors that enriches protein kinases from lysates based on the affinity of individual kinases for the different immobilized inhibitors, their kinase abundance, and the activation state of the kinase (Cooper et al., PLoS One, 2013, 8, e66755; Duncan et al., Cell, 2012, 149, 307-321; Kurimchak et al., Cell Reports, 2016, 16, 1273-1286; Stuhlmiller et al., Cell Reports, 2015, 11, 390-404; and Zawistowski et al., Cancer Discov., 2017, 7, 302). MIB/MS allows the measurement of the level and/or activity of kinases from all major kinome subfamilies with a large percentage representing the understudied kinome for which there are limited reagents for adequate studies. Kinase protein levels/activity are quantified in patient tumors using a newly designed super-SILAC kinome standard and subsequently interrogated in representative cancer cell lines for growth and survival functions using RNAi to establish kinase dependencies. Kinases are then evaluated as therapeutic targets alone or in combination with currently approved therapies or those in clinical trials. To define cancer-related function and biomarkers of kinase inhibition, a comprehensive proteomic interrogation using kinome profiling, phosphoproteomics, and single-run proteomes are performed on cancer cells depleted of kinases. Collectively, this strategy provides a proteomics workflow to identify candidate kinase targets directly from clinical tumor specimens, determine kinase vulnerabilities alone or in combination therapies, and establish the functional role of the kinase in cancer.

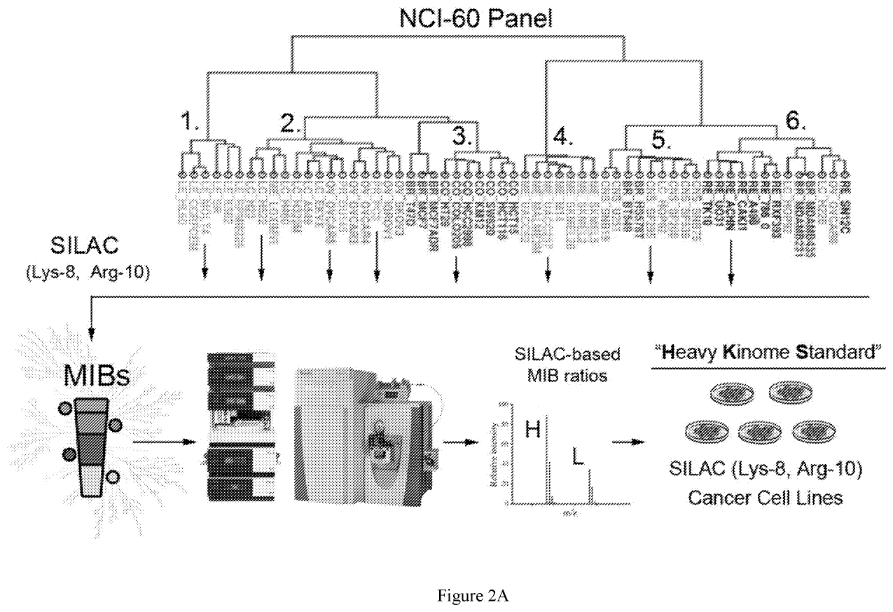

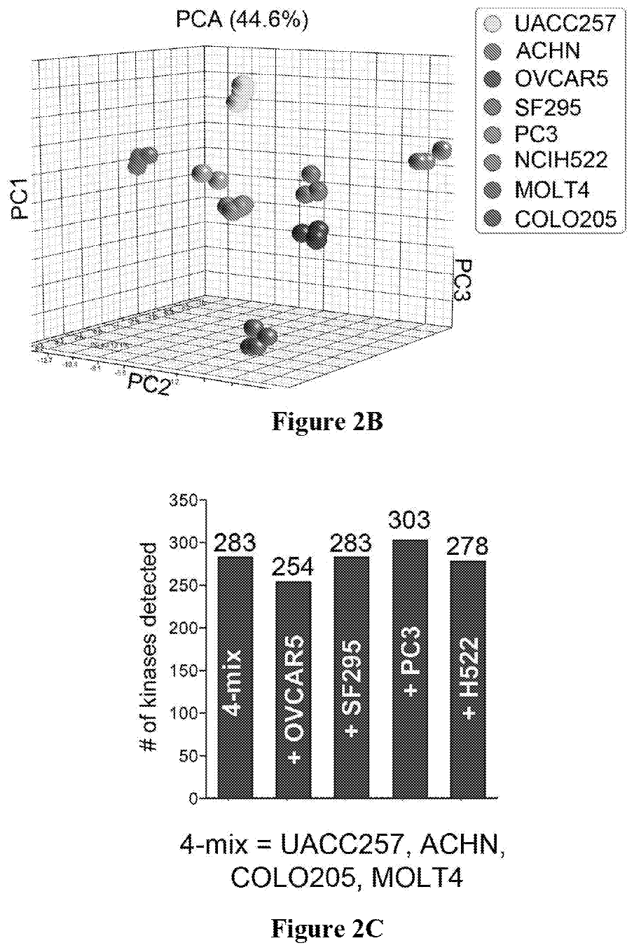

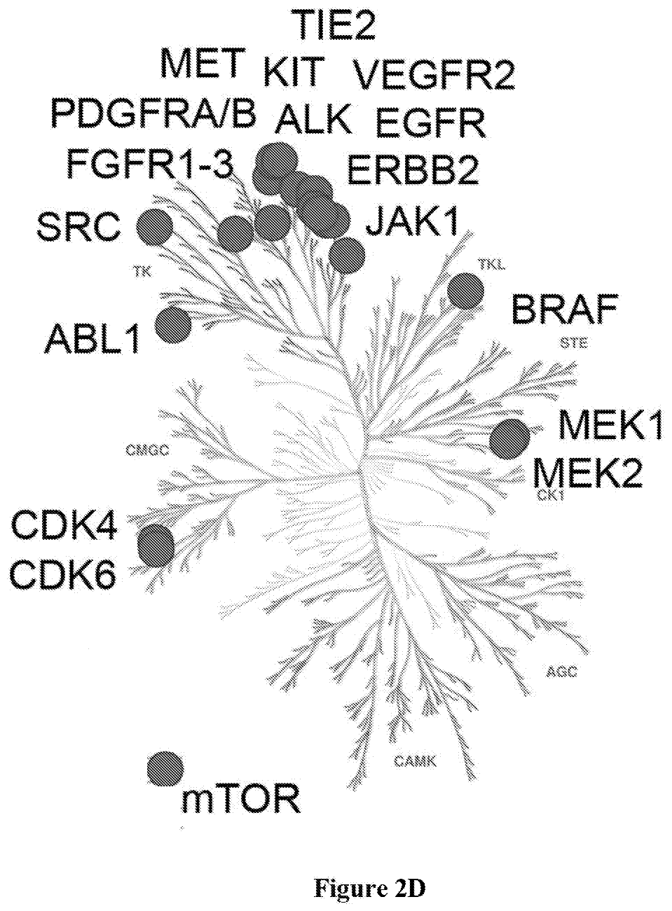

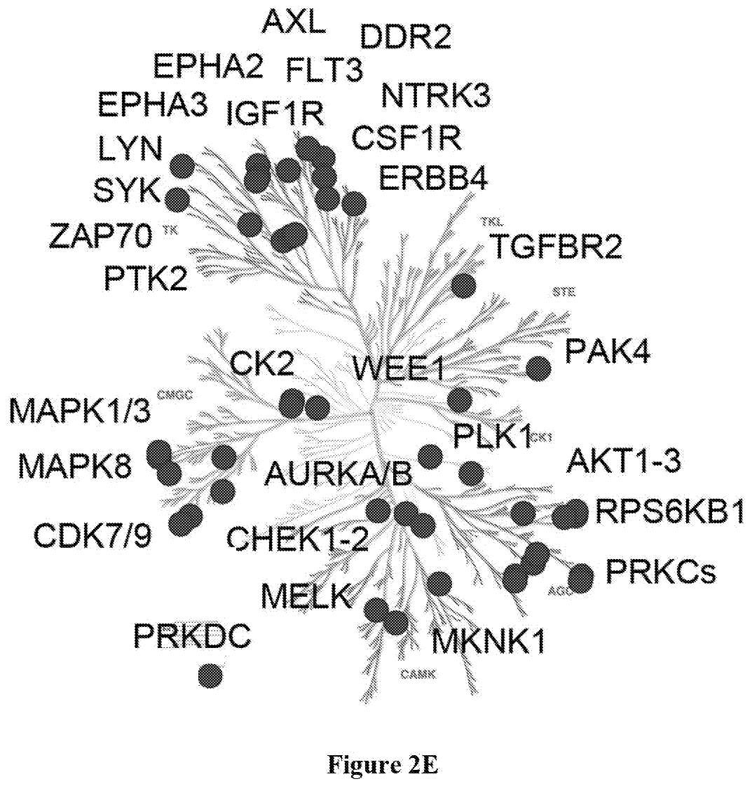

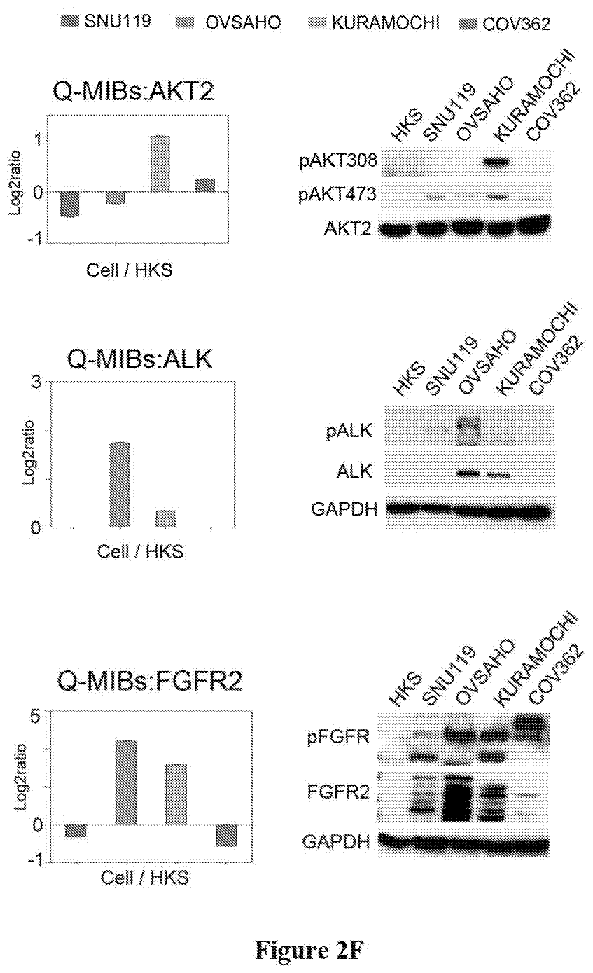

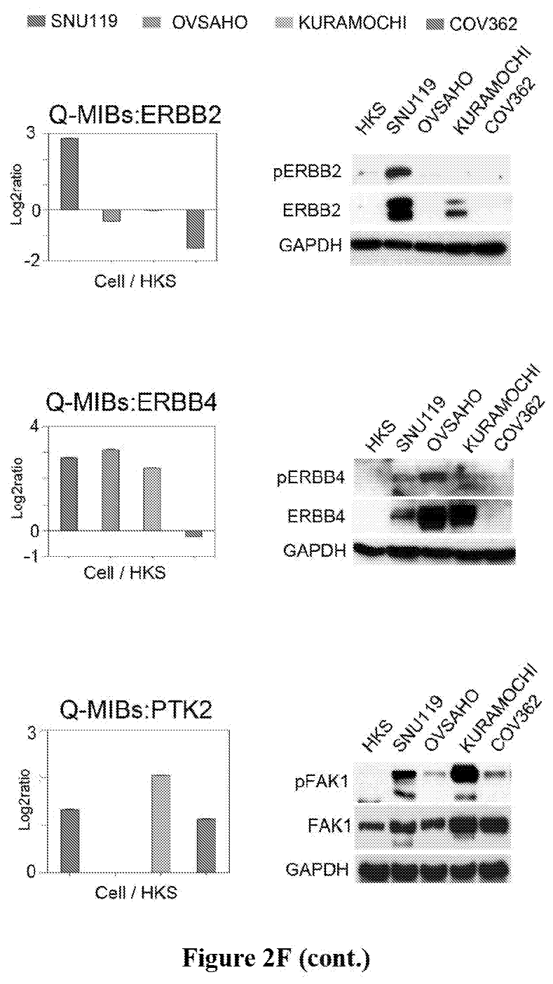

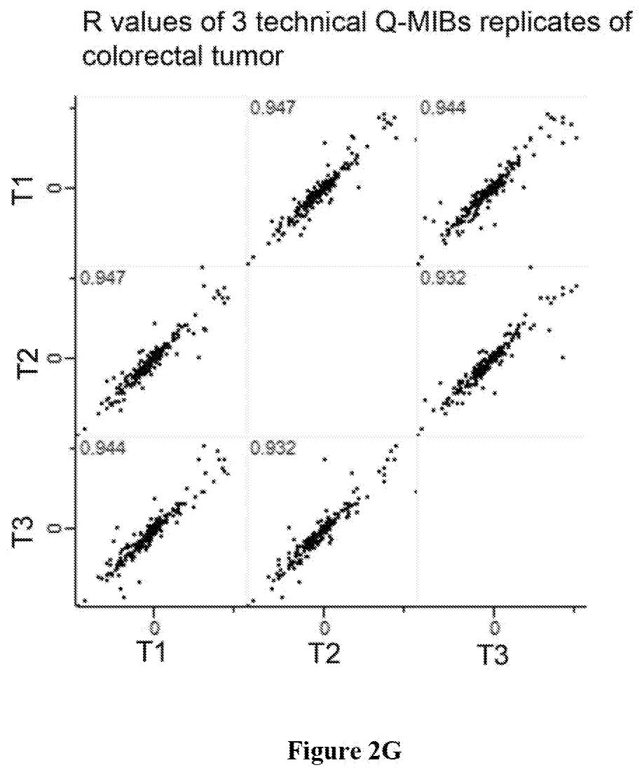

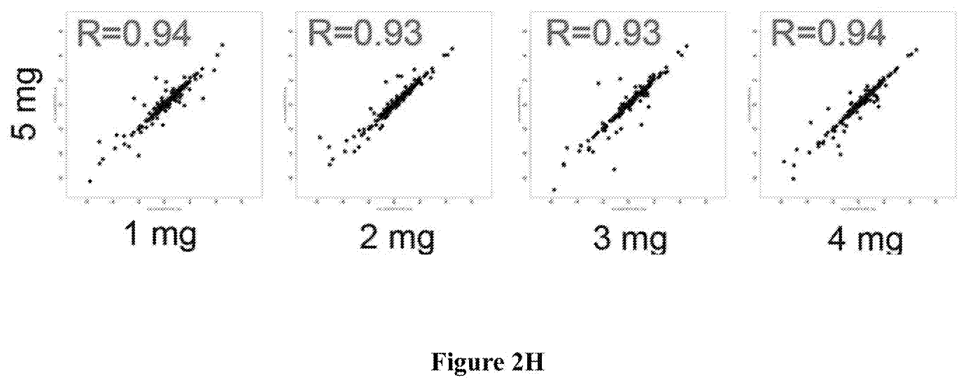

To quantify the kinome across large cohorts of tumor samples, a super-SILAC kinome standard method was designed consisting of a cocktail of cancer cell lines that encompasses the activity/level of the majority of the human kinome to be used as a control for individual samples. Eight cancer cell lines (UACC257, ACHN, OVCAR5, SF295, PC3, NCIH522, MOLT4, COL0205) were selected from the NCI-60 panel based on their diverse gene expression clustering (Gholami et al., Cell Reports, 2013, 4, 609-620), mutation and copy number status (Barretina et al., Nature, 2012, 483, 603-607), SILAC labeled and kinase levels/activity for each cell line determined using quantitative MIB/MS (see, FIG. 2A). The mixture of 5 cancer cell lines (UACC257, MOLT4, COL0205, ACHN and PC3) exhibited the most kinome diversity and was selected as the SILAC-based Heavy Kinome Standard (HKS) providing a universal (independent of cancer-type) ‘heavy’ kinome reference sample to be used repeatedly to quantify the kinome (see, FIGS. 1B, 2B, and 2C).

Comprehensive characterization of the kinome captured by Q-MIBs revealed nearly 70% (358/518) of the kinome can be SILAC-quantified with significant coverage of kinases with FDA approved drugs, those currently being investigated in clinical trials, and kinases not targeted but with established oncogenic driver function (see, FIGS. 1C, 1D, 2D, and 2E). It should be noted that most of the kinases quantified by Q-MIBs in tumors have no established cancer function and sparse (<100) number of publications, representing the understudied or truly ‘dark’ cancer kinome (see, FIGS. 1D and 1E). As the HKS contains an equal mixture of kinase(s) from the 5 cancer cell lines that are expressed or active at different levels, it provides a baseline or averaged kinome activity for samples to be referenced. Kinome profiling of 4 distinct HGSOC cell lines using QMIBs relative to HKS identified the unique elevated kinases from each cell line, which were confirmed by total and activation-dependent phosphorylation blots (see, FIG. 2F).

Measurement of kinases from tumors is highly reproducible across tumor replicates with R correlations values ≥0.9 comparing the same tumor kinome profiled on 3 separate days by Q-MIBs, as well as different concentrations of protein inputs (see, FIGS. 1F, 2G, and 2H). Additionally, the HKS can be propagated indefinitely, demonstrating high correlations in kinome quantitation across 3 distinct SILAC-kinome batches prepared months apart (see, FIG. 1F). Reproducibility of the Q-MIBs assay was confirmed using a model-based approach for assessing technical reproducibility and outlier detection (Anastassiadis et al., Nat. Biotech., 2011, 29, 1039-1045) (see, FIG. 2I).

Collectively, an equal amount of the HKS can be added to any type of non-labeled sample (normal tissue, tumors or cell lines) providing a robust proteomics strategy to quantify the majority of the kinome across large cohorts of samples, amongst treatments and importantly across time. Applying Q-MIBs, both targeted and dark kinome signatures that are distinct between tumor types or that are unique to a given cancer can be established. The targeted kinome signatures are immediately actionable through small molecules, while dark kinome signatures represent novel drug targets for cancer therapy and new avenues for cancer kinase research.

### Example 2: Kinome Profiling of HGSOC Uncovers Dark Kinase Signature

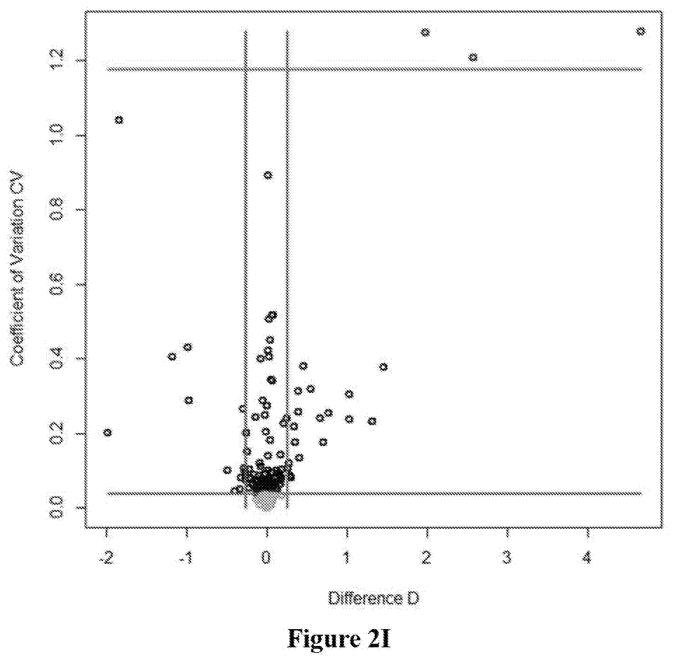

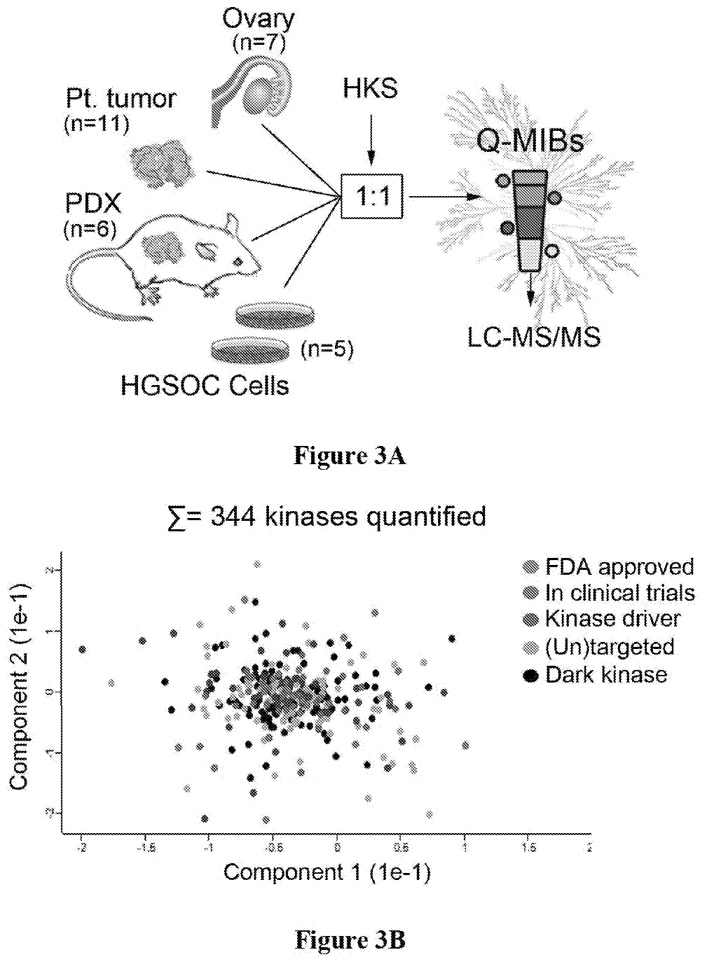

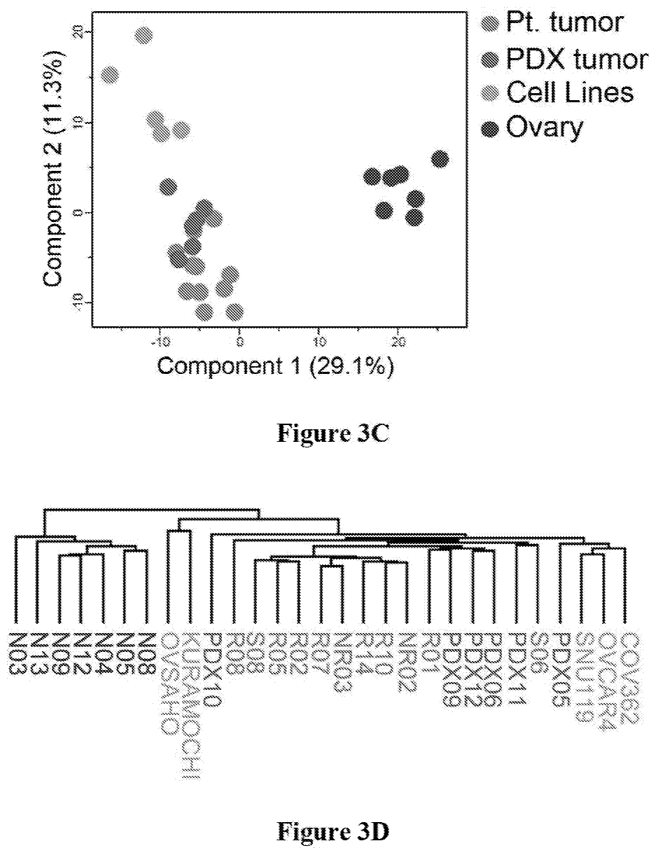

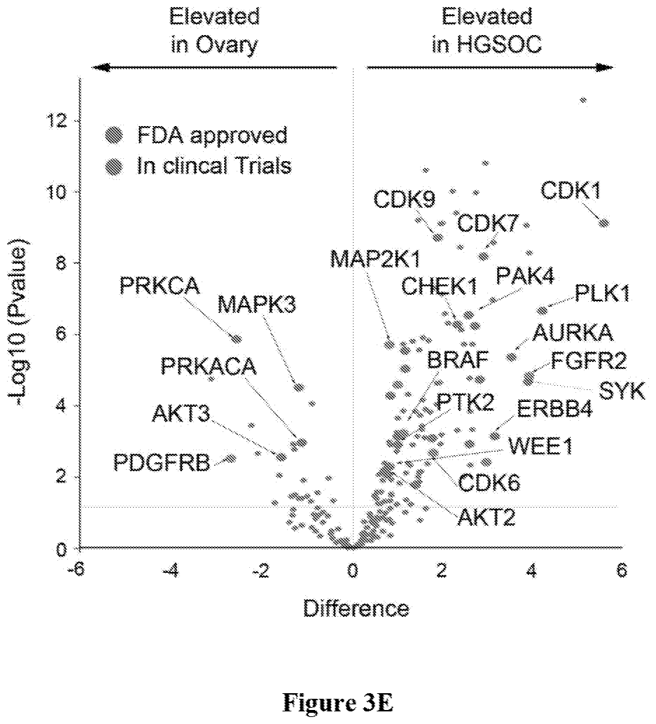

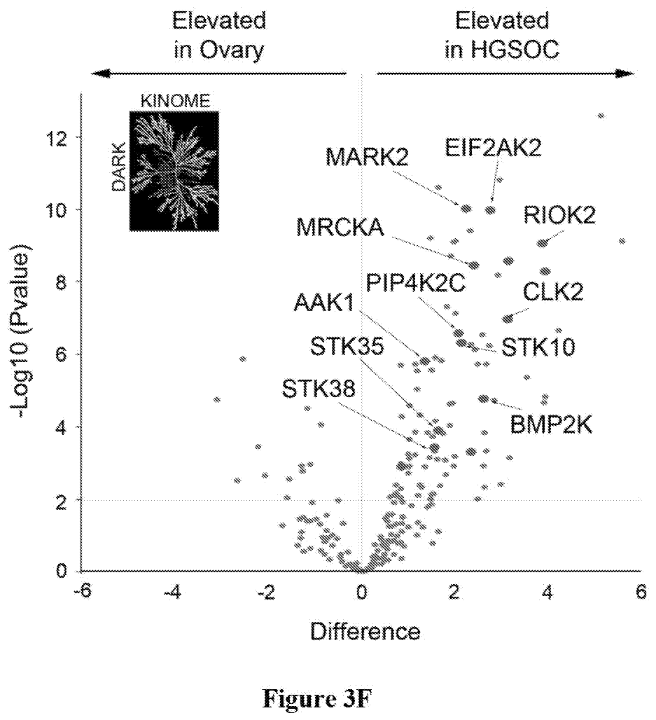

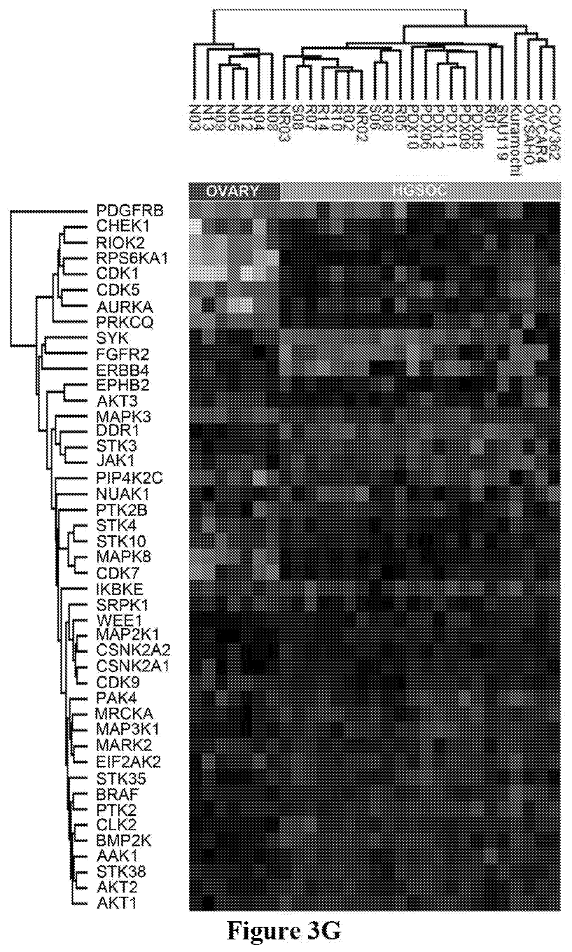

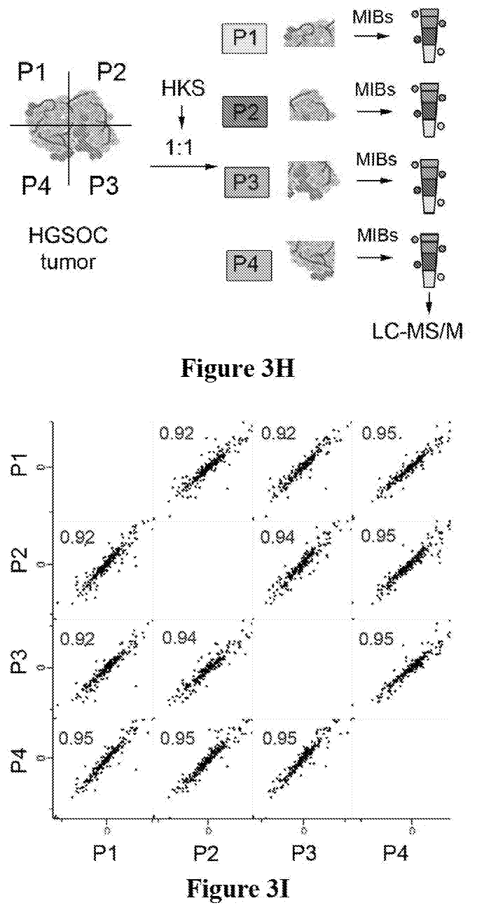

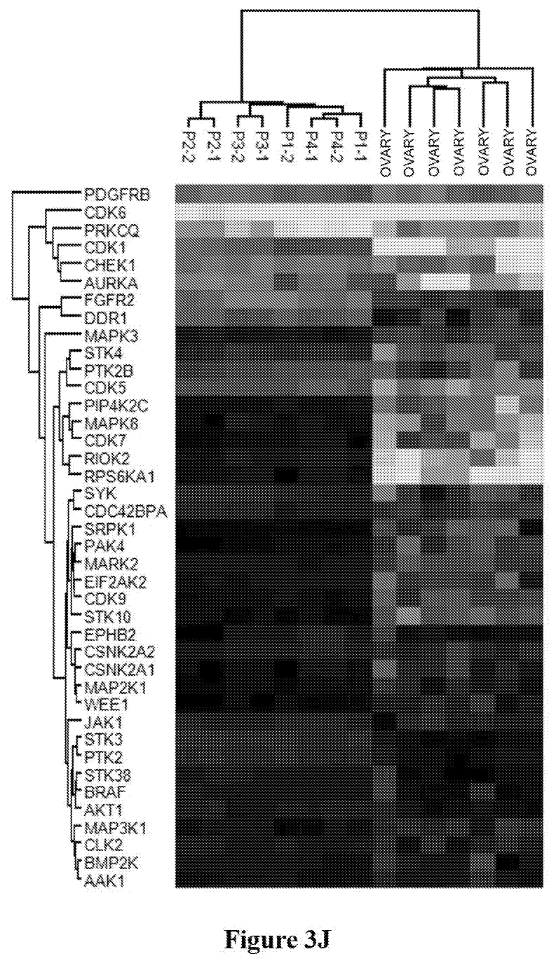

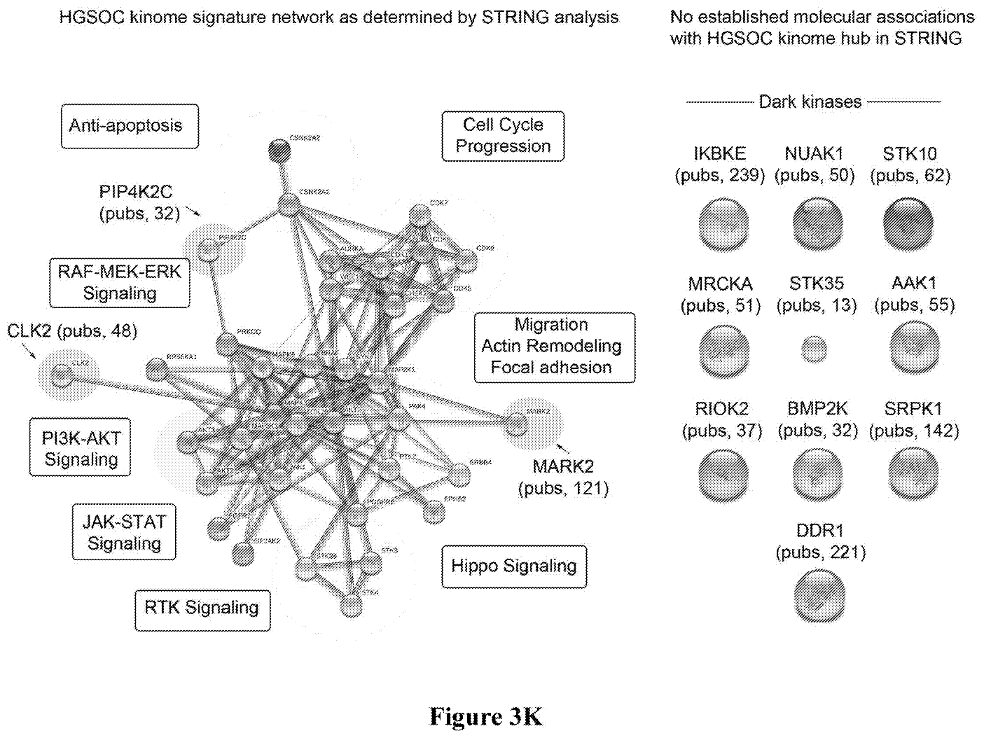

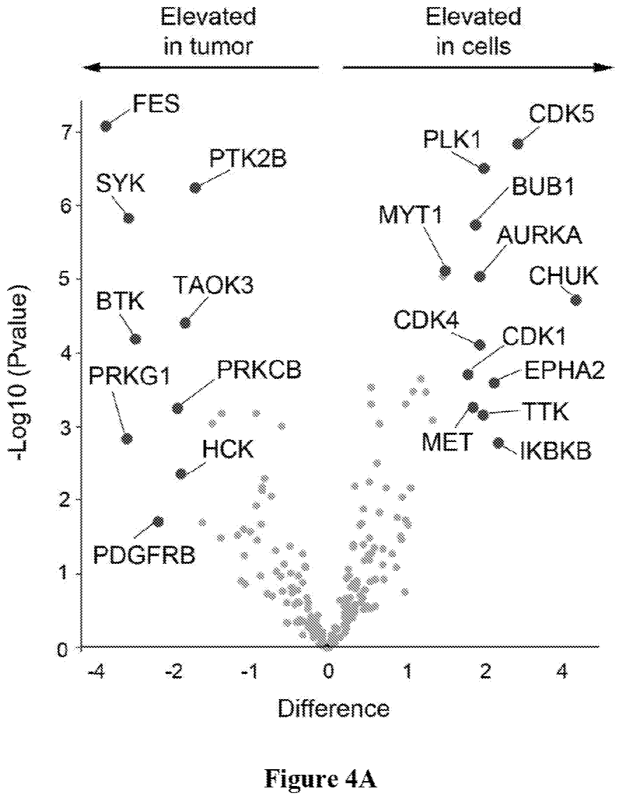

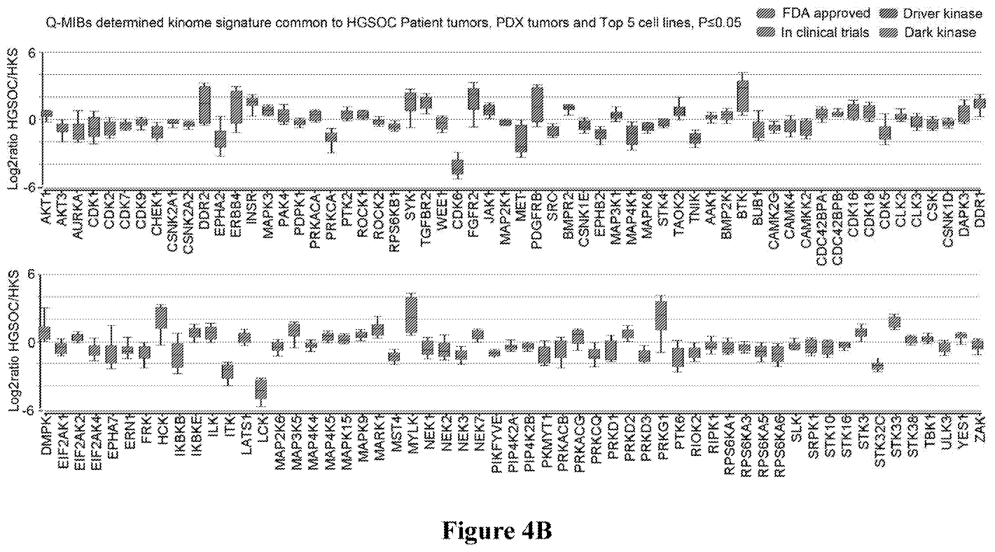

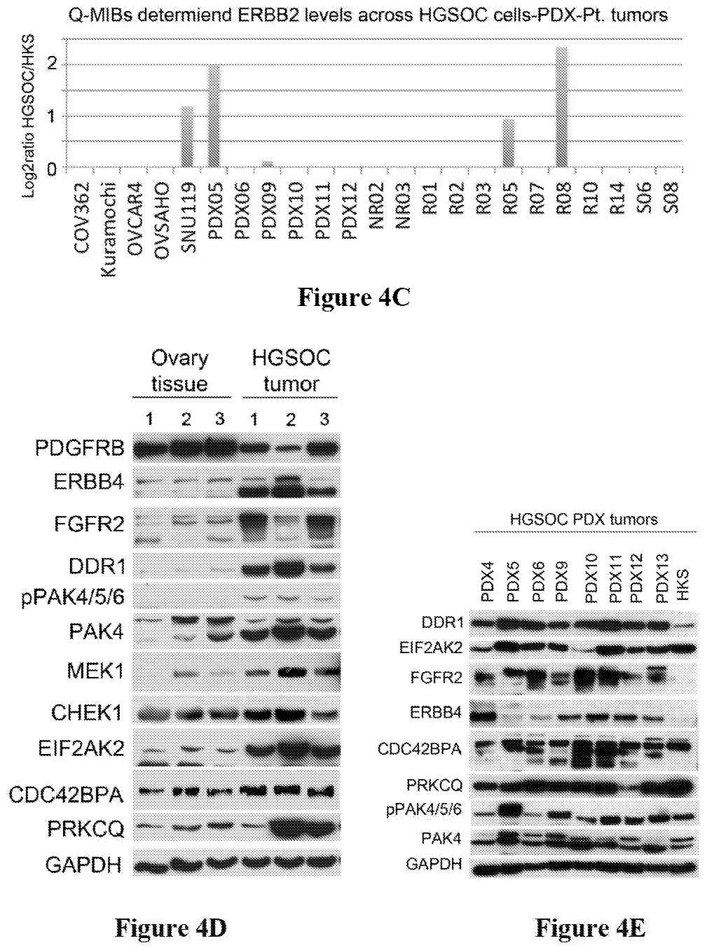

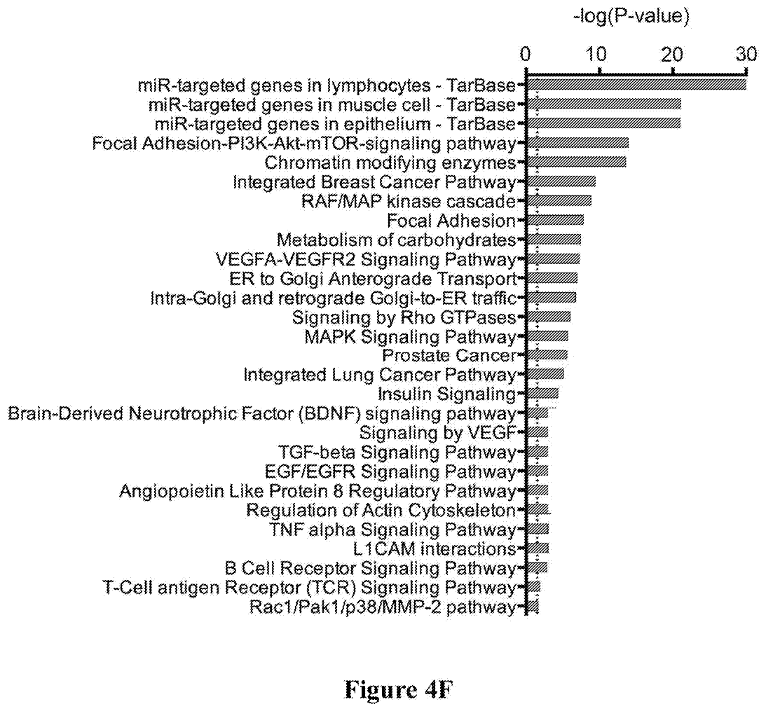

Treatment of HGSOC, the most common and deadly subtype of epithelial ovarian cancer, is currently lacking effective targeted kinase inhibitor therapies (Zhang et al., Cell, 2016, 166, 755-765). Genomic studies have identified frequent RAS/PI3K and/or RB/CDK signaling alterations in HGSOC patient tumors supporting therapies targeting kinases within these pathways). To identify kinase dependencies and therapeutic avenues in HGSOC, kinome profiling of HGSOC patient tumors (n=11), PDX tumors (n=6) and OC cell lines (n=5) relative to adjacent normal ovary tissue (n=7) was carried out using Q-MIBs (see, FIG. 3A, and Table 1). In total, 344 kinases were quantified by Q-MIBs, 19 that have FDA-approved inhibitors, 50 in clinical trials and the majority (80%) representing the (Un)targeted and dark kinome (see, FIG. 3B). Principal component analysis (PCA) and hierarchical clustering of kinome profiles demonstrates that the adjacent normal ovary tissue kinome(s) were distinct from both the tumors isolated from PDX models or directly from patients (see, FIGS. 3C and 3D). The PDX tumors and tumors isolated directly from patients showed high similarity in kinome profiles, confirming the HGSOC PDX tumors retain kinome architecture and activity profiles consistent with HGSOC patient tumor biopsies. The HGSOC cell line kinome(s) were distinct from adjacent normal ovary tissue, and showed diversity from ovarian tumors, where cell lines exhibited elevated cell cycle kinase activities (PLK1, CDK4 and AURKA etc.) relative to tumors likely due to higher growth rates of cells in culture (see, FIG. 4A). Additionally, tumors showed elevated stromal-like (i.e., PDGFRB) and blood-associated and/or immune kinases (BTK, HCK, SYK and FES) compared to HGSOC cell lines, consistent with the stromal microenvironment surrounding the tumor tissues. Statistical analysis of the kinome(s) across the HGSOC cells and tumors revealed a signature of kinases that shared common MIB-binding ratios relative to the control HKS, indicating these kinases exist at similar protein levels/activity in HGSOC (see, FIG. 4B). A number of kinases with FDA-approved or drugs in clinical trials showed common MIB-binding ratios relative to the HKS across samples, including, AKT1, DDR2, ERBB4, INSR, MAPK3, PAK4, PTK2 (FAK1), FGFR2, JAK1, ROCK1 and PDGFRB. However, most of the kinases with common MIB-binding ratios across HGSOC samples represented the (Un)targeted and dark ovarian cancer kinome with no drugs currently in clinical trials and/or established function in HGSOC.

TABLE 1
PDX tumors used f0r HGSOC profiling

| Q-MIB | | # of | mg | | |
|---|---|---|---|---|---|
| # | Case | kinases | used | Histology | Harvest Site |
| PDX05 | OC-14 | 251 | 5.000 | Cystadenocarcinoma | Peritoneal cavity |
| PDX06 | OC-42 | 244 | 5.000 | Serous | Ovary |
| | | | | Cystadenocarcinoma | |
| PDX09 | OC-1 | 223 | 5.000 | Adenocarcinoma, | Peritoneal cavity |
| | | | | NOS | |
| PDX10 | OC-16 | 230 | 5.000 | Serous | Ovary |
| | | | | Cystadenocarcinoma | |
| PDX11 | OC-49 | 253 | 5.000 | Serous | Peritoneal cavity |
| | | | | Cystadenocarcinoma | |
| PDX12 | OC-60 | 236 | 5.000 | Serous | Ovary |
| | | | | Cystadenocarcinoma | |

Volcano plot comparisons of Q-MIB-determined kinome profiles in HGSOC tumors relative to normal ovary tissue revealed a distinct kinome signature composed of a network of kinases previously identified as driver kinases in HGSOC that are currently being investigated in clinical trials for HGSOC including BRAF, MAP2K1, FAK1, AKT2, AURKA, CDK6, WEE1 and CHEK1 (see, FIG. 3E), as well as numerous kinases that have been linked to HGSOC but are not currently targeted in trials such as SYK, PAK4, FGFR2, ERBB4, DDR1, NUAK1, IKBKE and JAK1 (see, FIG. 3E). Elevated ERBB2 was detected by Q-MIBs in 2/12 patient tumors, 2/6 HGSOC PDX tumors and 1/5 HGSOC cell lines consistent with previous reports showing ERBB2 alterations occur at low frequency in HGSOC (Wilken et al., Future Med. Chem., 2012, 4, 447-469), which may reflect the poor clinical outcomes reported for anti-ERBB2 therapies in HGSOC (see, FIG. 4C). Many kinases distinct to HGSOC had no previous association or established function in HGSOC, representing the unexplored HGSOC dark kinome. Several dark kinases including AAK1, MRCKA (CDCl42BPA), STK38, EIF2AK2, MARK2, CLK2, STK10, BMP2K and STK35 displayed common Q-MIB-binding ratios across HGSOC tumors and were statistically distinct from normal ovary tissue, representing candidate therapeutic avenues for HGSOC (see, FIG. 3F). Altered protein levels in HGSOC patient tumors relative to normal ovary tissue of several Q-MIB-signature kinases including ERBB4, FGFR2, DDR1, PAK4, MEK1, CHEK1, EIF2AK2, PRKCQ, and MRCKA were confirmed by blot, as well as commonly detected across HGSOC PDX tumors (see, FIGS. 4D and 4E). Together, an HGSOC kinome signature was identified that consists of kinases with known driver function and several that have not been previously linked to HGSOC (see, FIGS. 3G and 4B).

Notably, the HGSOC kinome signature was highly correlated across sections of a HGSOC tumor with correlations >0.9, suggesting that a common kinome signaling network may predominate HGSOC tumors (see, FIGS. 3H, 3I, and 3J). STRING analysis of the HGSOC kinome signature revealed a large signaling network that included established kinase drivers that promote cancer through their role in anti-apoptosis, cell cycle progression, hippo signaling, focal adhesion, actin cytoskeleton remodeling (migration, invasion), RAF-MEK-ERK, P14-PI3K signaling and RTK signaling (see, FIG. 3K). Furthermore, global phosphoproteomics characterization of HGSOC tumors revealed enrichment of signaling pathways involved in focal adhesion-PI3K-AKT-mTOR, RAF/MAP kinase cascade, focal adhesion and actin cytoskeleton (see, FIG. 4F and data not shown). This proteomic analysis of patient tumors supports ongoing clinical trials in HGSOC targeting kinases within these pathways, as well as implicates additional kinases such as FGFR2, ERBB4, PAK4 and JAK1 as potential signaling hubs and therapeutic targets in HGSOC. Notably, dark kinases CLK2, PIP4K2C and MARK2 were linked to established driver kinase pathways, while IKBKE, NUAK1, STK10, MRCKA, DDR1, AAK1, RIOK2, BMP2K, SRPK1 and STK35 were not functionally associated with the HGSOC kinase network in STRING analysis, signifying kinases that are highly prevalent in HGSOC tumors with an unknown function in ovarian cancer (see, FIG. 2K).

### Example 3: Functional Interrogation of the Q-MIBs-Defined HGSOC Tumor Kinome Signature Using Established HGSOC Cell Lines

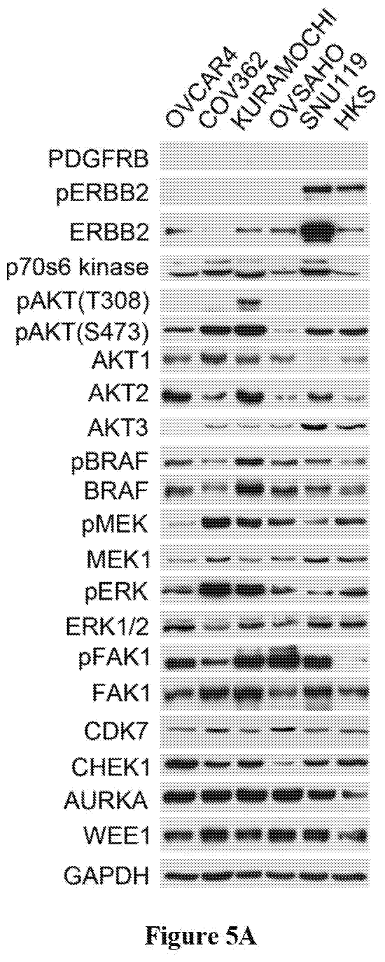

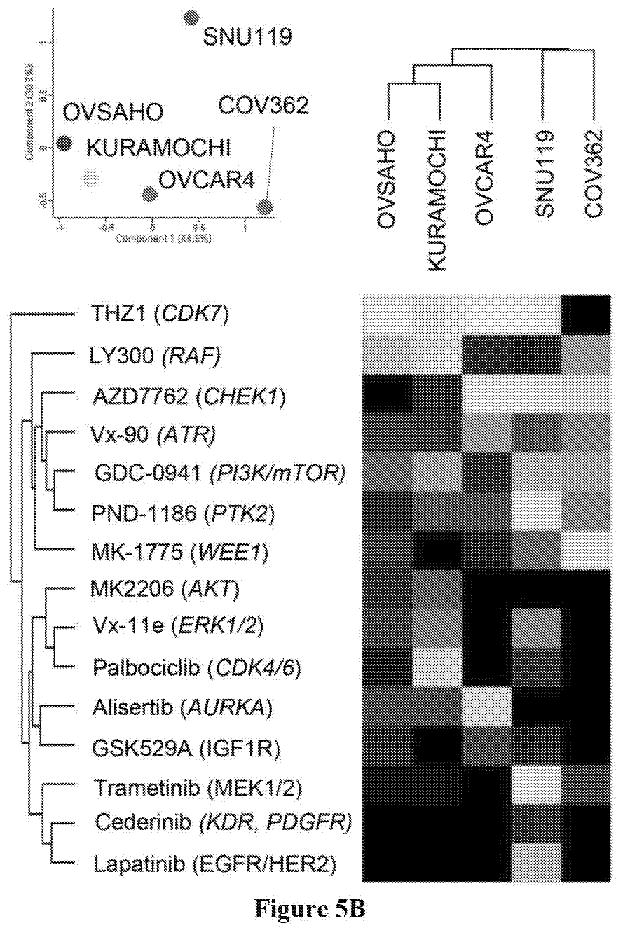

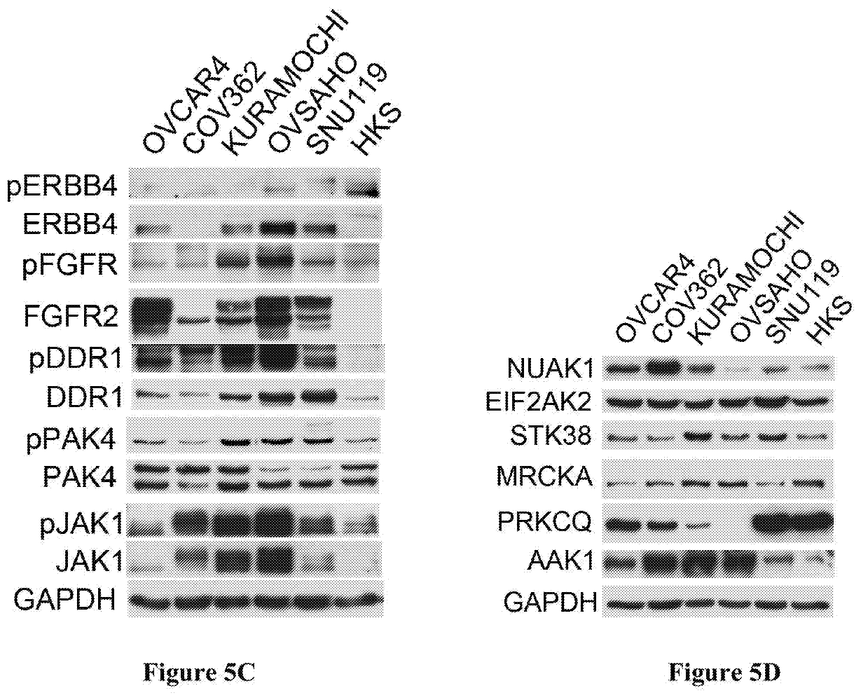

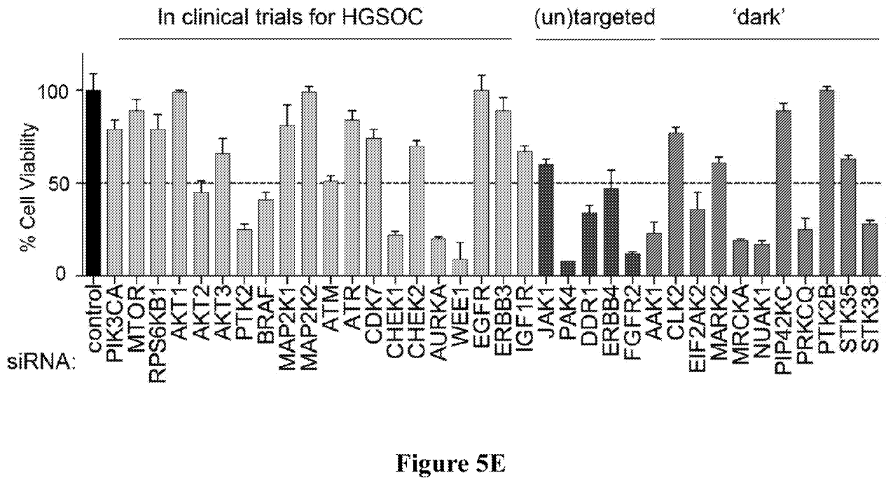

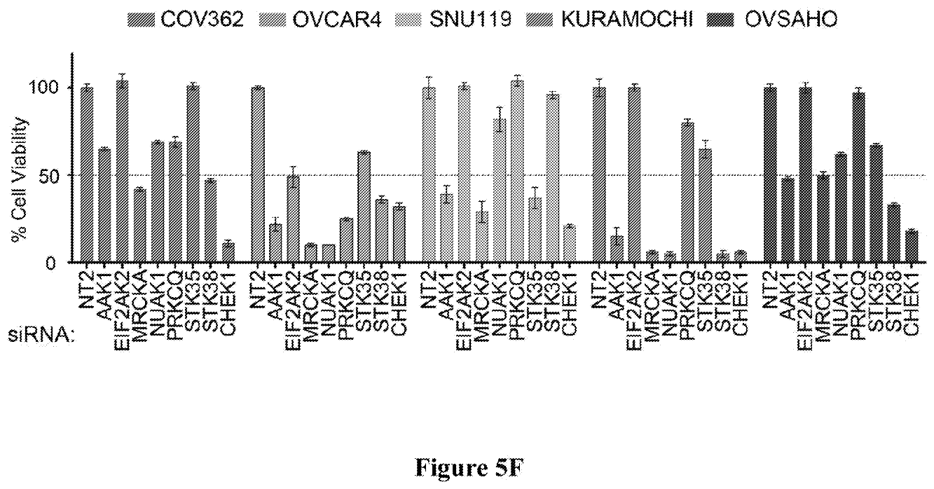

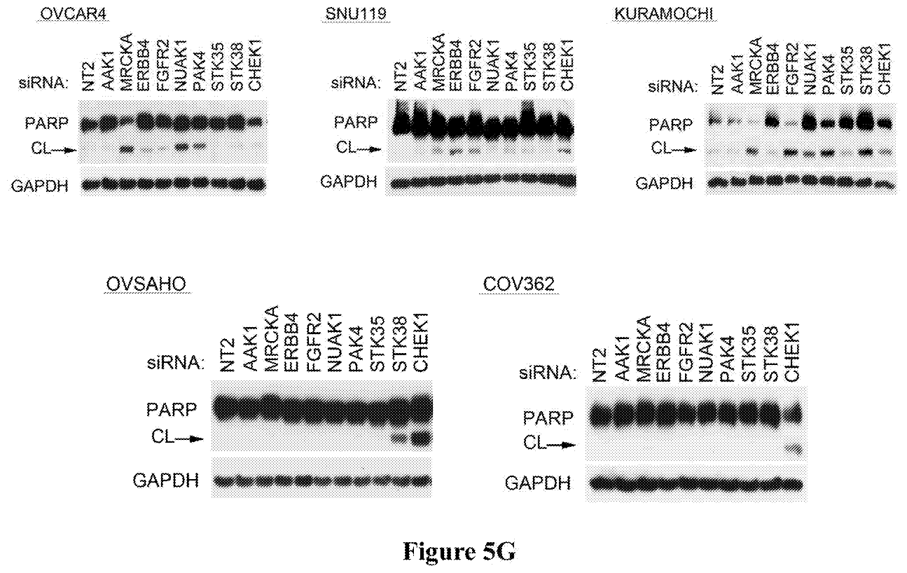

To explore the therapeutic potential of the HGSOC kinome signature, a detailed molecular analysis of protein levels/activity, growth and survival functions was carried out in a panel of recently validated HGSOC cell lines (Coscia et al., Nat. Commun., 2016, 7, 12645) that harbor commonly encountered HGSOC mutations and genetic rearrangements (see, FIG. 5A). Consistent with Q-MIBs signatures of HGSOC tumors, western blot analysis of kinases currently being targeted in HGSOC clinical trials showed HGSOC cells commonly displayed FAK1, BRAF, MEK, ERK, P70S6 kinase and AKT pathway activation, showed elevated AURKA and WEE1 protein levels relative to HKS and expressed CHEK1 and CDK7 (see, FIG. 5A). Activated ERBB2 was detected only in SNU119; while PDGFRB expression was not detected in HGSOC cells and AKT3 was observed at reduced levels relative to HKS, with exception of SNU119 cells. Targeted small molecule inhibition of kinases with established clinical-grade inhibitors revealed that although HGSOC cells show high correlation (R≥0.5) to patient HGSOC tumors at the genomic level (Coscia et al., Nat. Commun., 2016, 7, 12645), it is apparent that the HGSOC cell lines exhibit distinct kinase inhibitor vulnerabilities. PCA and hierarchical clustering of growth inhibition by kinase inhibitors currently in clinical trials showed that OVSAHO, KURAMOCHI and OVCAR4 are more like each other in their response to kinase inhibition than COV362 or SNU119 (see, FIG. 5B and Table 2). The majority of HGSOC cells showed sensitivity towards inhibitors targeting WEE1, CHEK1, CDK7, ATR, FAK1, BRAF and PI3K/MTOR, while inhibitors targeting AKT, CDK4/6, MEK, ERK, AURKA, IGF1R, EGFR/HER2 or pan-TK showed more diversity in efficacy (see, FIG. 3B). Notably, CDK7 inhibition had a profound impact on HGSOC cell growth in all but COV362 cells consistent with recent global phosphoproteomics studies that identified CDK7 as a driver kinase in HGSOC (Francavilla et al., Cell Reports, 2017, 18, 3242-3256). SNU119 cells harboring elevated ERBB2 levels cells showed exquisite sensitivity to EGFR/HER2 demonstrating that although ERBB2 activation occurs at low frequency in HGSOC, therapies targeting ERBB2 could be quite effective in ERBB2 altered HGSOC patients.

TABLE 2
Kinase inhibition profile of HGSOC cells.

| Target | DRUG | uM | COV362 | OVCAR4 | Kuramochi | OVSAHO | SNU-119 |
|---|---|---|---|---|---|---|---|
| HER2 | Lapatinib | 1.2 | 0.9972868 | 0.97400804 | 0.97035888 | 1.05286409 | 0.15 |
| TK | Cederinib | 1.2 | 0.92458697 | 1.08904763 | 0.94659508 | 1.08937543 | 0.62578969 |
| FAK1 | PND1186 | 1.2 | 0.34 | 0.51 | 0.51 | 0.69 | 0 |
| RAF | LY300 | 1.2 | 0.26117834 | 0.60615023 | 0.09150801 | 0.1660417 | 0.65993143 |
| PAK | PF03758039 | 1.2 | 0.49159459 | 0.20629652 | 0.47828733 | 0.65897916 | 0.35047132 |
| CHEK | AZD7762 | 0.312 | 0.05871983 | 0 | 0.66302506 | 0.79876715 | 0 |
| IKKB | TPCA-1 | 1.2 | 1.09410673 | 0.87676159 | 0.9879665 | 0.98265706 | 0.9374945 |
| CDK7 | THZ1 | 0.156 | 1.02649557 | 0 | 0.07068872 | 0 | 0 |
| IGF1R/INS | GSK529A | 5 | 0.97313752 | 0.52261182 | 0.97323881 | 0.65735566 | 0.65789815 |
| JAK | Cerdulatinib | 2.5 | −0.0082561 | 0.50511738 | 1 | 1 | 0.83951828 |
| AURKA | Alisertib | 0.312 | 1.07532612 | 0.10163631 | 0.51990691 | 0.53867747 | 1.05716189 |
| EGFR | AZD4547 | 5 | 0.54739457 | 0.59890887 | 0.7095103 | 0.43510262 | 0.73798801 |
| CDK4/6 | Palbociclib | 5 | 0.92001235 | 0.79522514 | 0.09321069 | 0.69555662 | 0.58667165 |
| ATR | VX-90 | 0.625 | 0.3345537 | 0.26455546 | 0.60826901 | 0.58851136 | 0.5100428 |
| PI3K/MTOR | GDC-0941 | 0.625 | 0.21969402 | 0.62331307 | 0.23611229 | 0.42929716 | 0.17522128 |
| WEE1 | MK-1775 | 0.312 | 0.03494295 | 0.6746769 | 1.11061819 | 0.59351948 | 0.43117134 |
| MEK | Trametinib | 0.0125 | 0.57662217 | 0.84358044 | 0.77082588 | 0.78240666 | 0 |
| MET | BMS777607 | 1.2 | 0.90788446 | 1.06227181 | 1.00725539 | 1.10170278 | 1.02800319 |
| AKT | MK2206 | 0.625 | 0.82540303 | 0.97710917 | 0.47009179 | 0.62326606 | 0.97279063 |
| ERK1/2 | Vx-11e | 0.625 | 1.01374726 | 0.96228894 | 0.35960483 | 0.548822 | 0.33591199 |

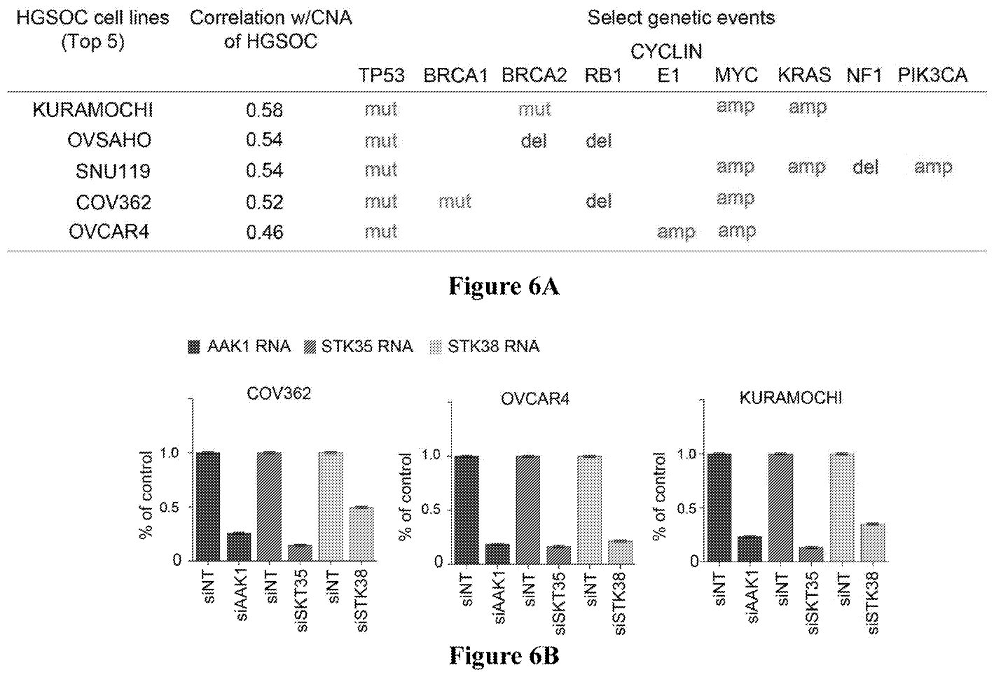

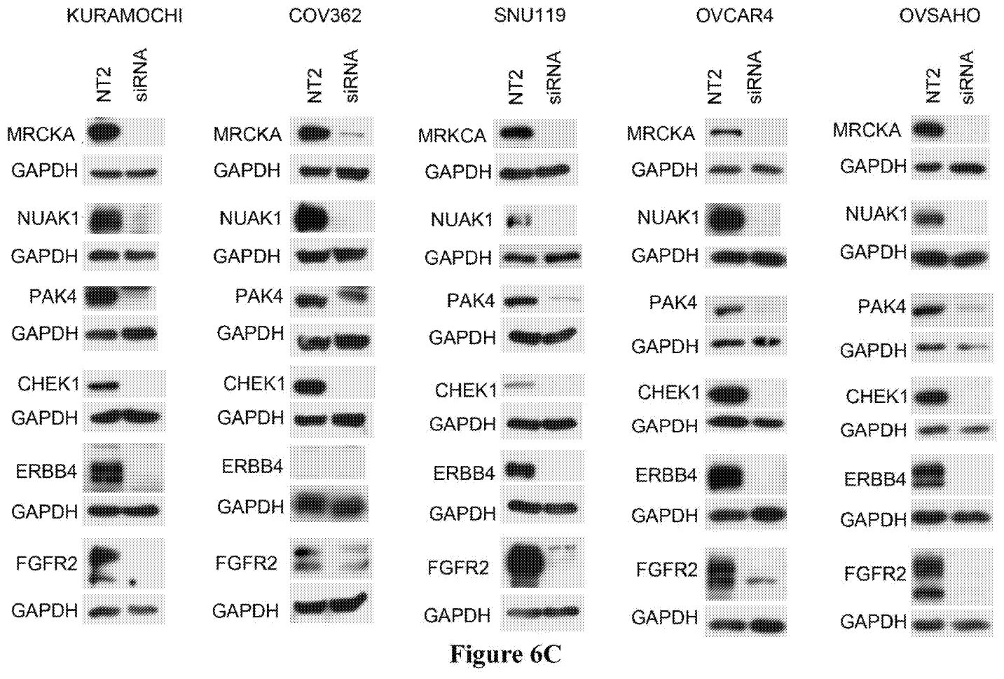

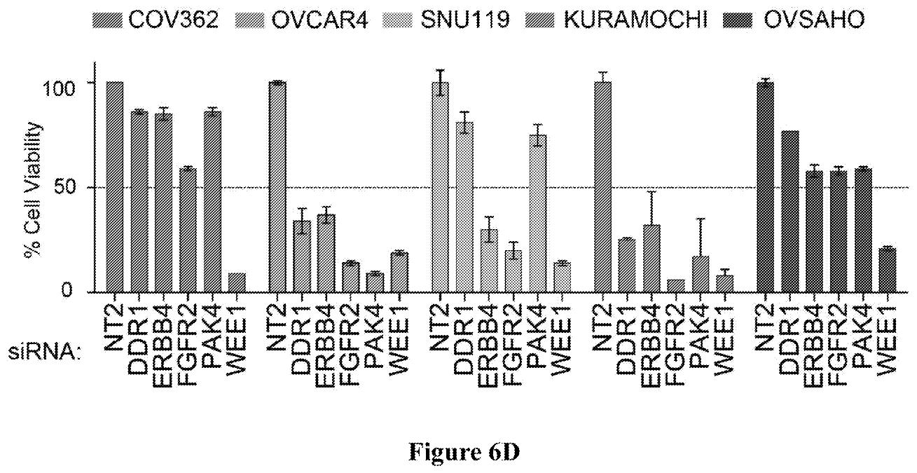

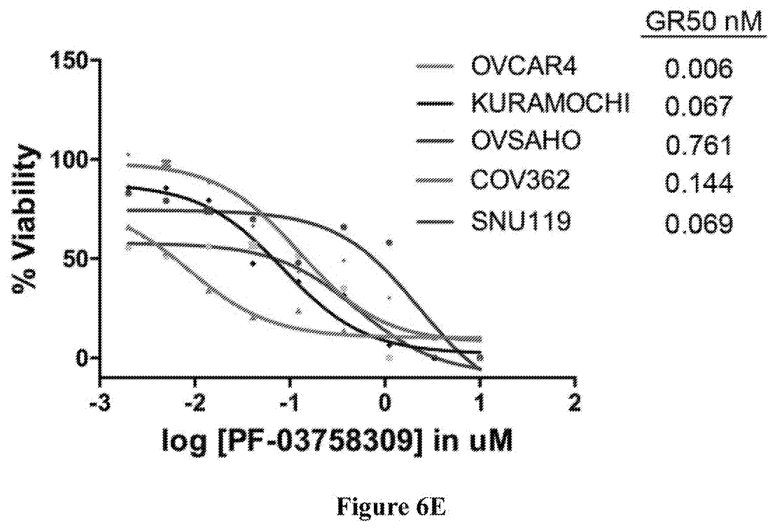

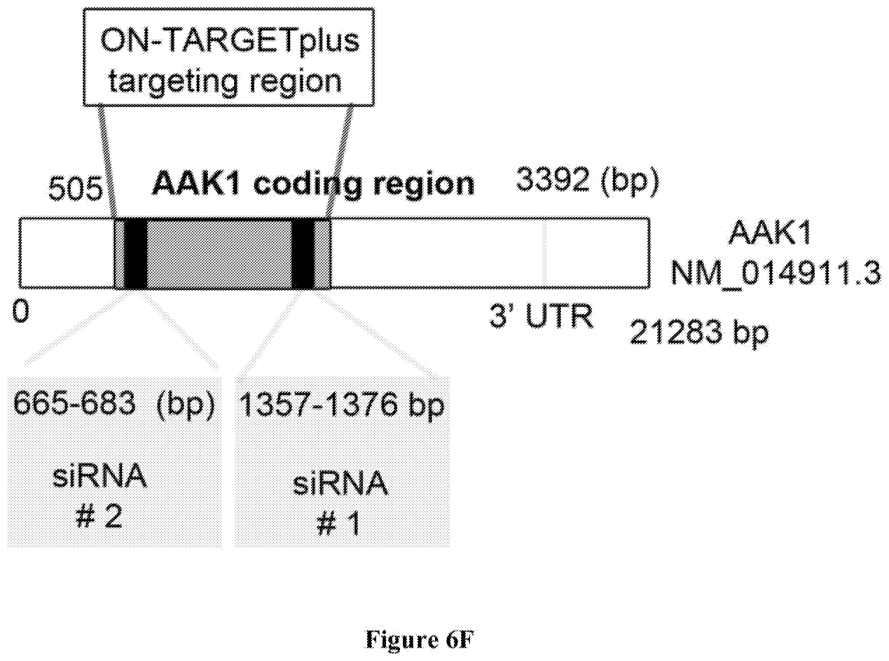

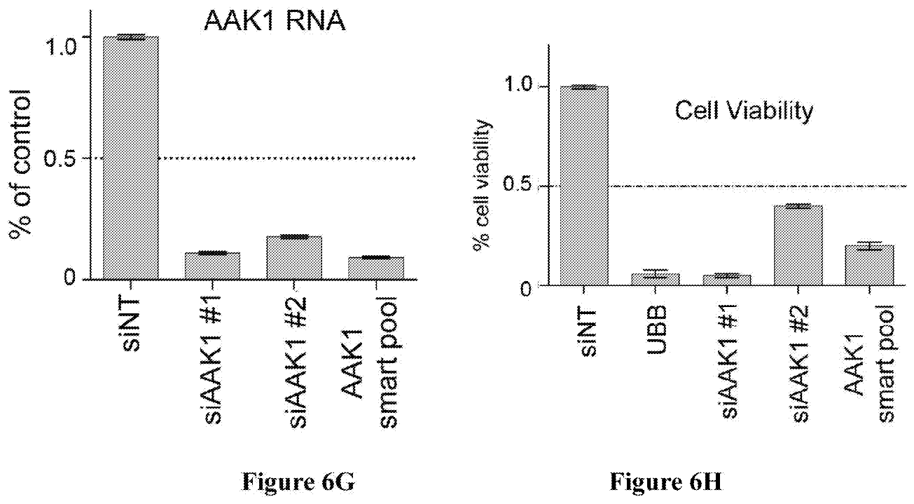

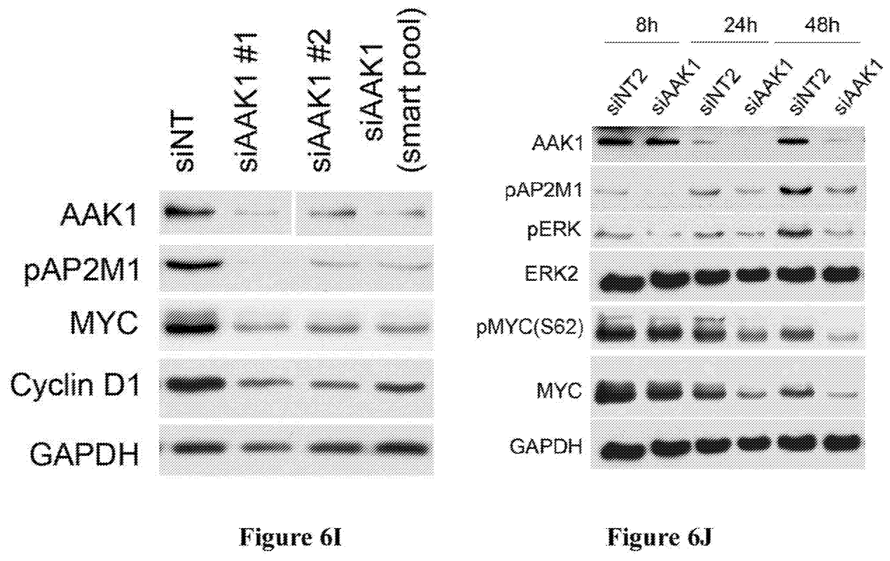

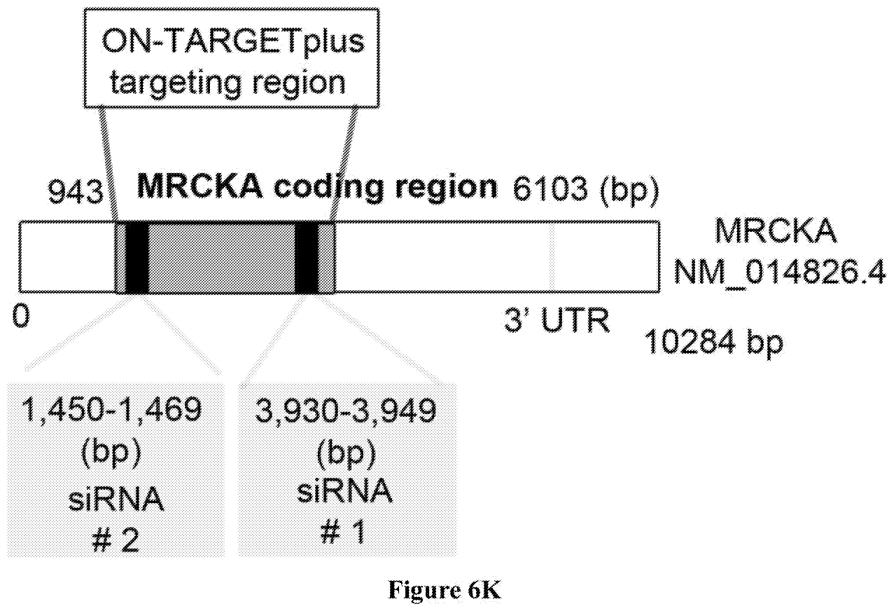

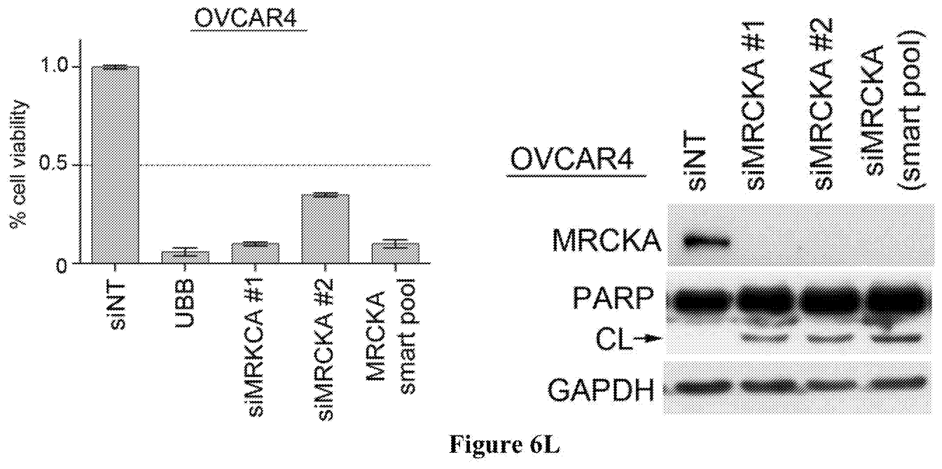

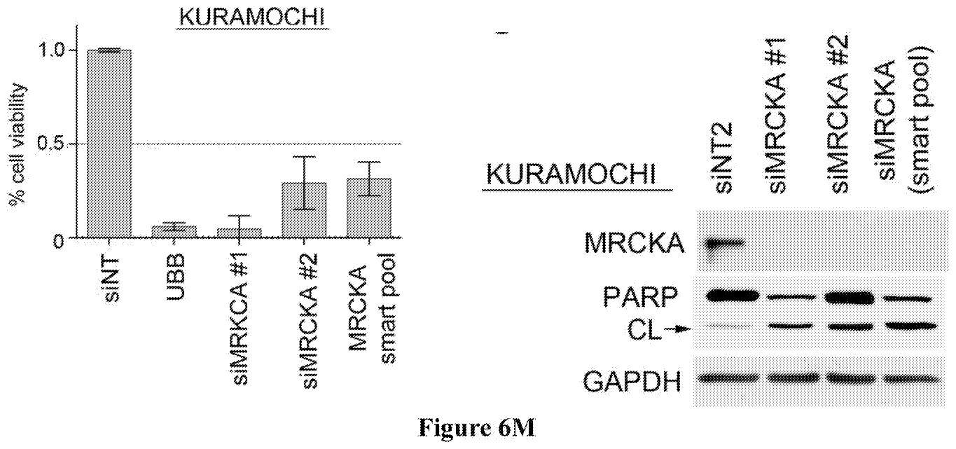

Further characterization of Q-MIB signatures in HGSOC cells by blot revealed FGFR2, ERBB4, DDR1, and PAK4 pathway activation, kinases that are currently being evaluated in clinical trials though not for HGSOC (see, FIG. 5C). Dark kinases NUAK1, EIF2AK2, STK38, MRCKA, PRKCQ and AAK1 were also detected by blot in all HGSOC cell lines, verifying Q-MIBs signatures (see, FIG. 5D). To define the growth and survival functions of Q-MIBs signature kinases not currently being evaluated in clinical trials for HGSOC, a candidate-driven RNAi-mediated knockdown screen was carried out in OVCAR4 cells (see, FIG. 5E). siRNA-mediated knockdown of DDR1, ERBB4, FGFR2 or PAK4 and dark kinases AAK1, MRCKA, NUAK1, PRKCQ, and STK38 blocked cell growth >50% in OVCAR4 cells similarly or to a greater extent than established HGSOC kinase drivers (CHEK1, AURKA, WEE1, AKT2, BRAF, or FAK1), further supporting these kinases as novel candidate drug targets for HGSOC. In contrast, knockdown of other kinases such as EGFR, ERBB3, PIP4K2C and PTK2B had little effect on the proliferation of these cells, signifying OVCAR4 cells do not rely on these kinases for growth. Knockdown of kinases in the HGSOC cells was confirmed by blot and/or RT-PCR (see, FIGS. 6B and 6C), and cell viability and apoptosis assessed following siRNA-mediated depletion of candidate kinases across the HGSOC cell line panel (see, FIGS. 5F, 5G, and 6D). Genetic depletion of DDR1, FGFR2 or ERBB4 inhibited cell growth in KURAMOCHI and OVCAR4 cells >50%, SNU119 cells were growth inhibited by FGFR2 or ERBB4 knockdown, while OVSAHO and COV362 were unaffected (see, FIG. 6D). FGFR2 or ERBB4 knockdown induced apoptosis in OVCAR4, SNU119 and KURAMOCHI cells. PAK4 knockdown reduced cell viability >50% and induced apoptosis only in KURAMOCHI and OVCAR4 cells, demonstrating the dependency of these cell lines for PAK4 and highlighting PAK4 as a promising kinase target in HGSOC. Small molecule inhibition of PAK4 using PF-3758309 reduced cell viability to the greatest extent in OVCAR4 and KURMAOCHI cells, further illustrating PAK4 as a promising therapeutic target in HGSOC cells dependent on PAK4 for survival (see, FIG. 6E).

NUAK1 knockdown reduced cell viability and induced apoptosis in OVCAR4 and KURAMOCHI cells, while STK38 blocked cell growth in OVSAHO, OVCAR4 and KURAMOCHI and induced apoptosis in OVSAHO and KURAMOCHI cells. COV362 cells were resistant to apoptosis following kinase knockdown except for CHEK1 depletion. Notably, genetic depletion of MRCKA reduced cell viability in all HGSOC cells and induced apoptosis in OVCAR4, KURAMOCHI and SNU119 cells, while AAK1 knockdown blocked cell growth in OVCAR4, KURAMOCHI, OVSAHO, or SNU119 and to lesser extent in COV362 cells but did not induce apoptosis. Knockdown of AAK1 or MRCKA with 2 distinct siRNAs that target different regions of the kinase genes confirmed on-target growth and survival effects observed by siGENOME siRNA pools (see, FIGS. 6F, 6G, 6H, 6I, 6J, 6K, 6L, and 6M). Knockdown of AAK1 with distinct siRNAs reduced cell viability >50% in OVCAR4 cells, inhibited AAK1 substrate AP2M1 phosphorylation and decreased MYC and Cyclin D1 protein levels, supporting a role for AAK1 in promoting HGSOC cell proliferation (see, FIGS. 6G, 6H, and 6I). Moreover, a time-dependent reduction in MYC protein levels and reduced ERK activity were observed following AAK1 knockdown in OVCAR4 cells, supporting a role for AAK1 in ERKMYC signaling in these HGSOC cells (see, FIG. 6J). Knockdown of MRCKA using 2 distinct siRNAs reduced cell viability and induced apoptosis in both OVCAR4 and KURAMOCHI cells confirming the dependency of these HGSOC cells on MRCKA protein for survival (see, FIGS. 6K, 6L, and 6M).

Collectively, small molecule inhibition of kinases currently being evaluated in clinical trials showed varying efficacy on HGSOC cell lines, highlighting the differences in kinase dependencies amongst cells that share common genomic alterations. Furthermore, genetic interference with dark kinases, such as MRCKA, effectively blocked cell growth in HGSOC cells, similar to targeting established HGSOC kinase drivers, supporting these previously unexplored kinases as potential new drug targets for HGSOC.

### Example 4: Proteomic Characterization of MRCKA Knockdown Reveals a Role in Established HGSOC Oncogenic Pathways

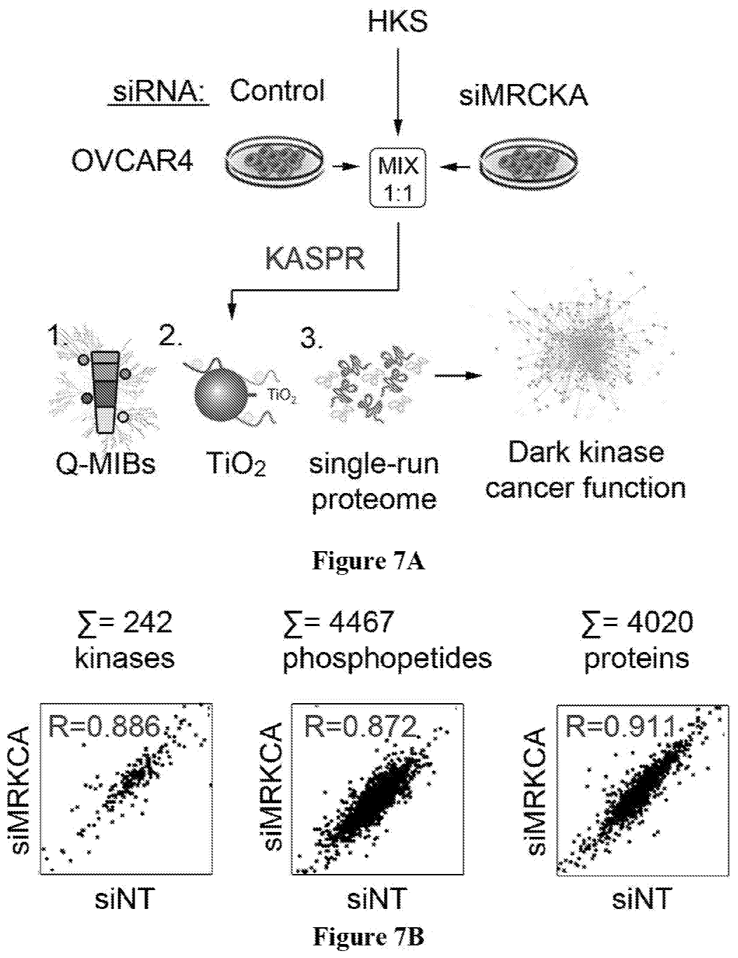

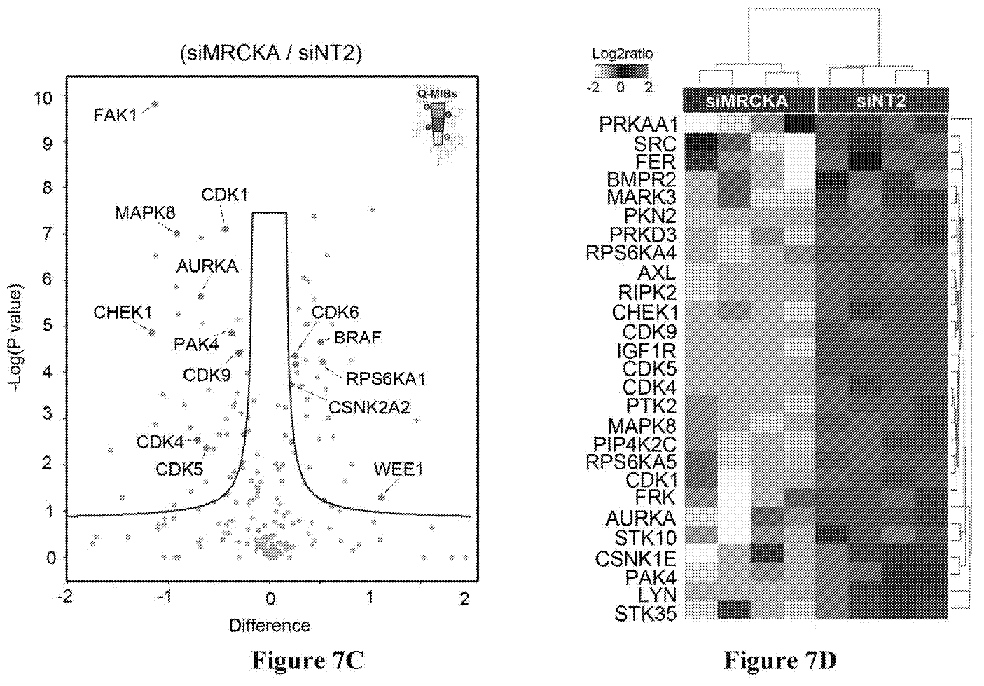

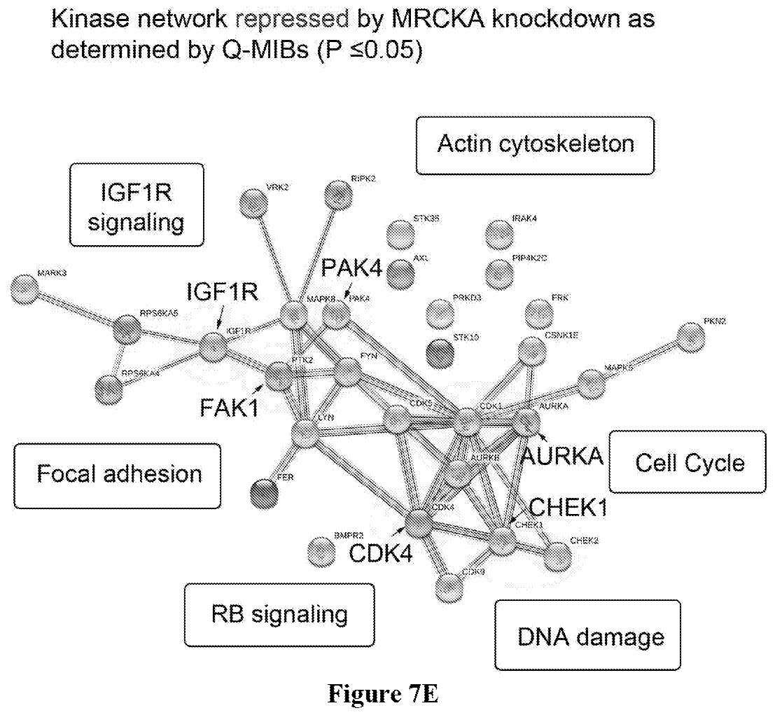

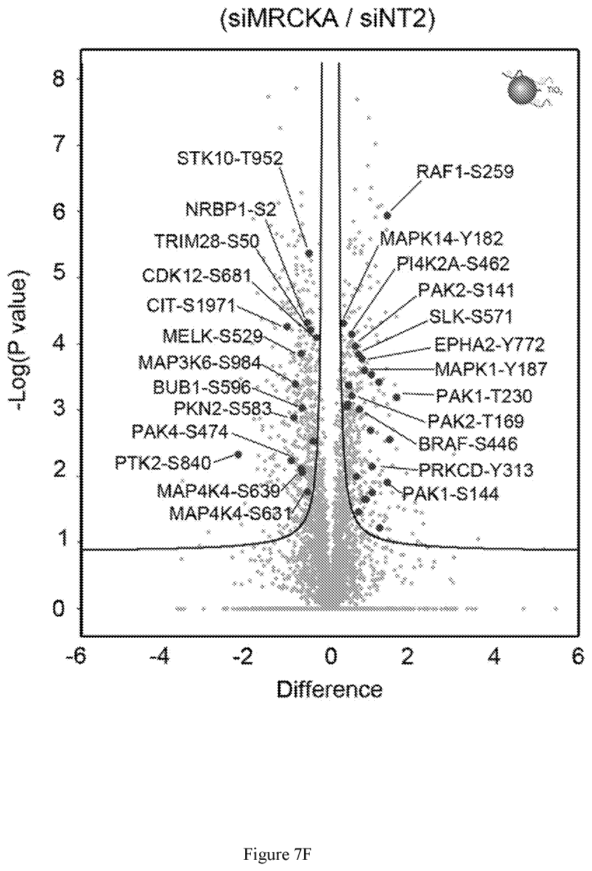

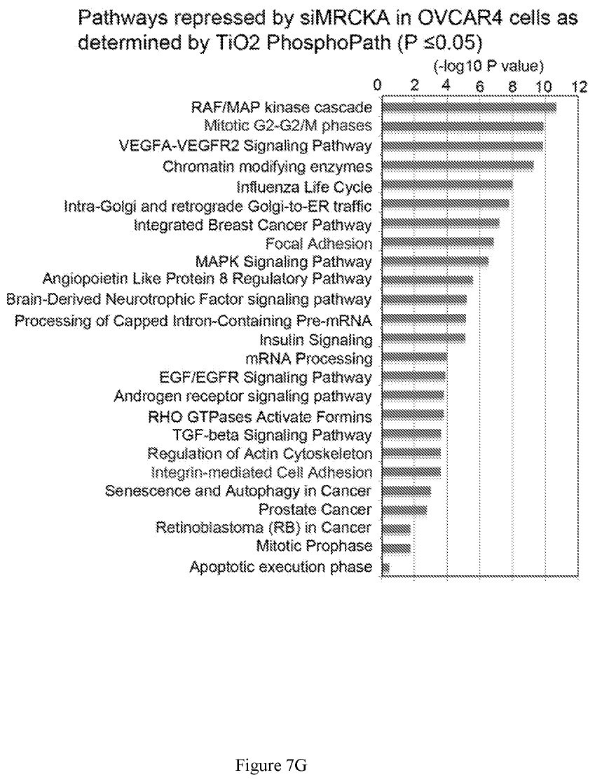

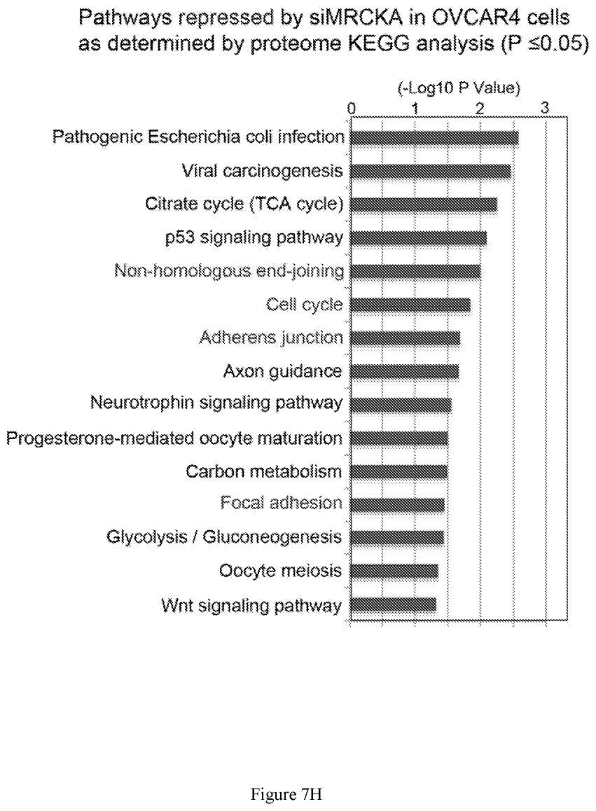

Myotonic dystrophy kinase-related Cdc42-binding kinase alpha (MRCKA) has been shown to play a role in the regulation of cytoskeleton reorganization and cell migration, however, the functional role of MRCKA in HGSOC has not been established (Unbekandt and Olson, J. Mol. Med. (Berl), 2014, 92, 217-225; and Zhao et al., Small GTPases, 2015, 6, 81-8). To explore the oncogenic role of MRCKA in HGSOC, a detailed proteomic analysis of HGSOC cells was carried out following MRCKA knockdown using Kinome Activation Signature and Phosphoproteome Response (KASPR) profiling (see, FIG. 7A). KASPR profiling combines: 1) kinome profiling using Quantitative Multiplexed Inhibitor Beads (Q-MIBs) technology to selectively enrich and measure activity/level of endogenous protein kinases; 2) global phosphoproteomics analysis to define kinase and kinase substrate phosphorylation; and 3) deep single-run proteome analysis to define global proteome levels. KASPR profiling provides a more accurate molecular portrait of cancer signaling pathways directly or indirectly controlled by dark kinase of interest.

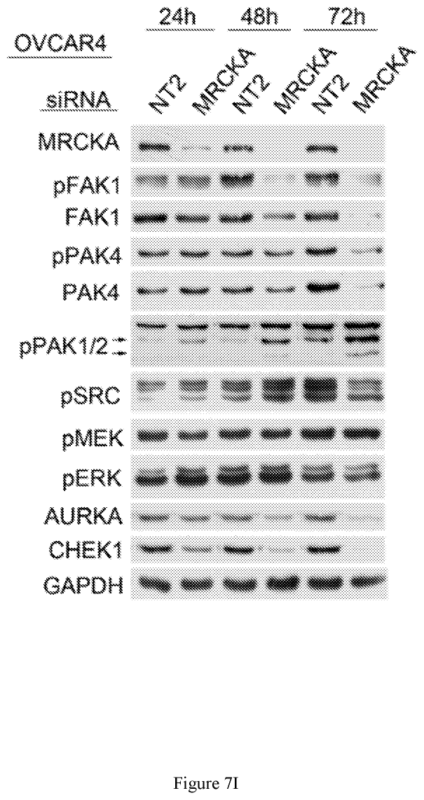

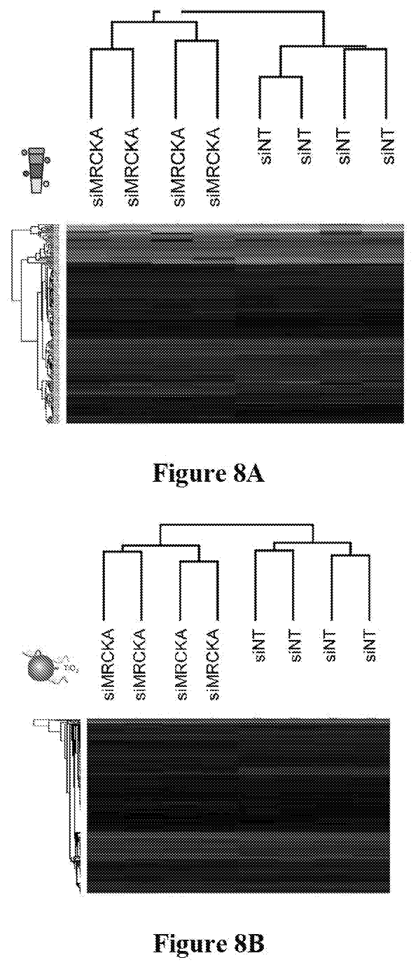

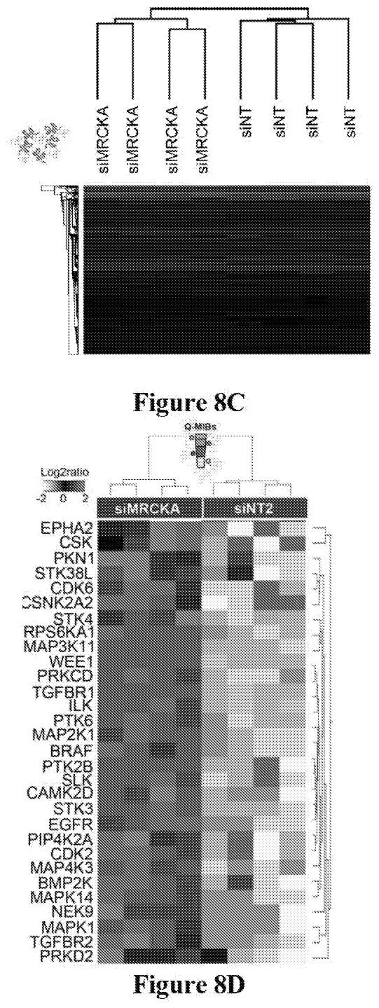

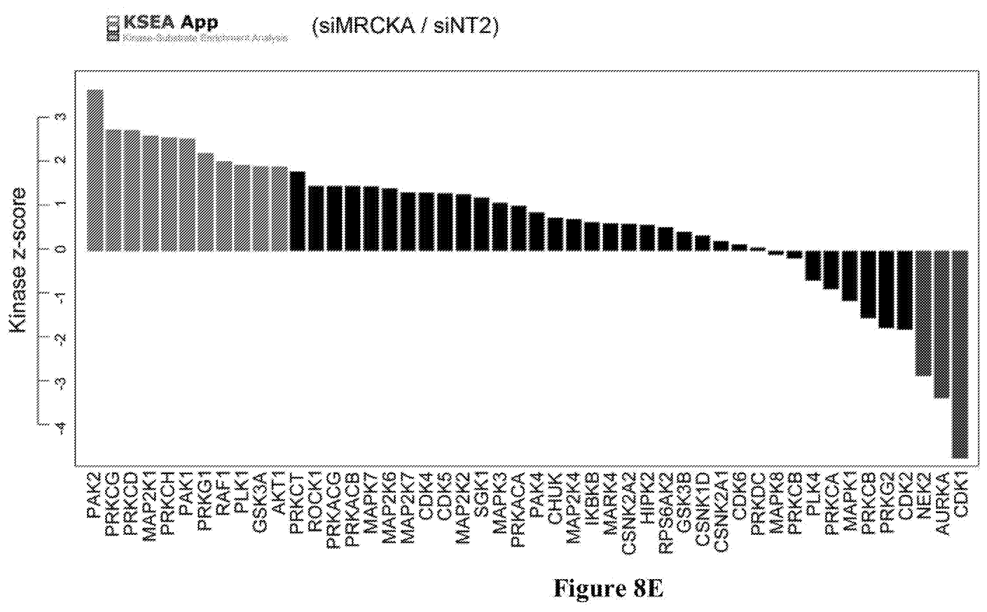

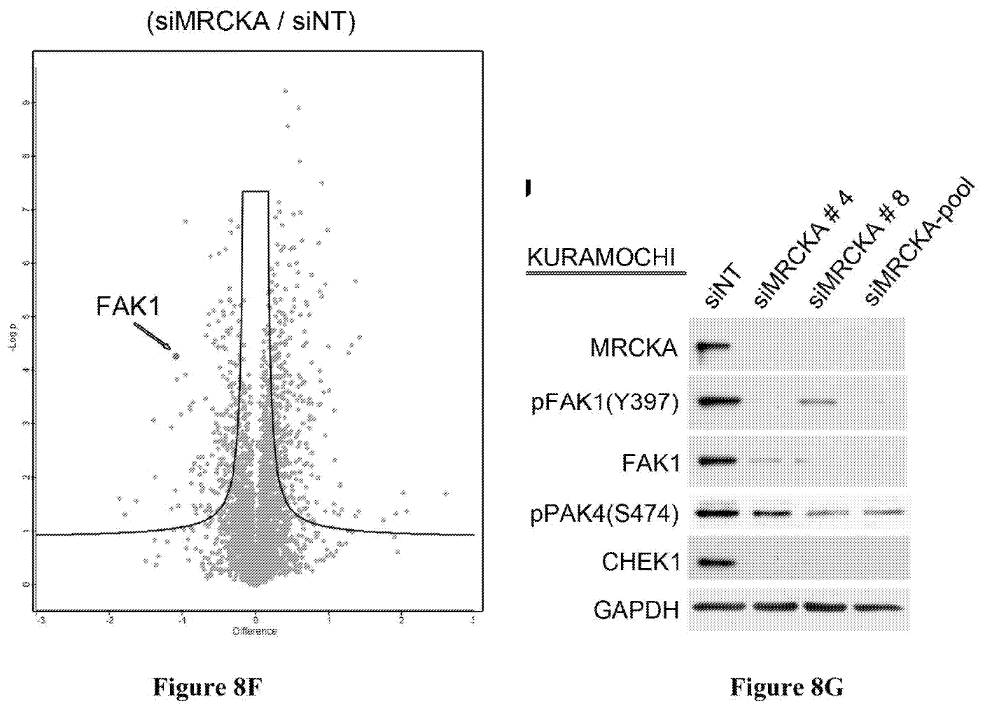

Knockdown of MRCKA reprogrammed the kinome, phosphoproteome, and proteome of OVCAR4 HGSOC cells distinctly from the control non-targeting siRNA, as shown by Pearson Correlation (R) and hierarchical clustering (see, FIGS. 7B, 8A, 8B, and 8C). Knockdown of MRCKA for 48 hours in OVCAR4 cells led to both induced and repressed kinase MIB-binding of a significant portion of the kinome (see, FIG. 7C). Notably, depletion of MRCKA protein in OVCAR4 cells resulted in reduced MIB-binding of established kinase drivers previously shown to promote HGSOC growth and survival including SRC, CHEK1, CDK4, IGF1R, FAK1, CDK1, AURKA, PAK4 and JAK1 (see, FIG. 7D). Increased MIB-binding of kinases including MAPK1, BRAF, MAPK14, EPHA2 and WEE1 was also observed following MRCKA knockdown, demonstrating loss of MRCKA protein results in both induction and repression of kinase signaling in HGSOC cells (see, FIGS. 7C and 8D). STRING analysis of kinases repressed by MRCKA knockdown, revealed a network involved in IGF1R signaling, actin cytoskeleton, focal adhesion, cell cycle, DNA damage and RB signaling (see, FIG. 7E). Consistent with Q-MIBs analysis, phosphoproteomics analysis of MRCKA knockdown cells showed reduced phosphorylation of PTK2 and PAK4, as well as increased phosphorylation of MAPK1, BRAF, PAK1 and PAK2 (see, FIG. 7F). Furthermore, Kinome Substrate Enrichment Analysis (KSEA) of phosphoproteomics datasets predicted inhibition of CDK1, AURKA and NEK2 and activation of PAK2, PAK1, PLK1, MAP2K1 and GSK3A following MRCKA knockdown (see, FIG. 8E). PhosphoPath analysis of phosphorylation events repressed by MRCKA knockdown revealed down regulation of signaling pathways involved in mitotic G2-G2/M phases, focal adhesion, regulation of actin cytoskeleton, integrin-mediated cell adhesion and retinoblastoma (RB) in cancer pathways (see, FIG. 7G). KEGG analysis of global proteome runs identified reduced pathways involved in p53 signaling, non-homologous end joining, cell cycle, metabolism and Wnt signaling. Notably, proteins involved in focal adhesion and adherence junction signaling including FAK1, were reduced in cells depleted of MRCKA further supporting a role for MRCKA in focal adhesion signaling in HGSOC (see, FIGS. 7H and 8F).

A time-dependent reduction in activating phosphorylation of FAK1 (Y397) and PAK4 (S474), as well as reduced protein levels of FAK1, PAK4, AURKA and CHEK1 following MRCKA knockdown were confirmed by western blot (see, FIG. 7I). Reduced FAK1 activating phosphorylation and FAK1 and CHEK1 protein levels were detected using 2 distinct MRCKA siRNAs (see, FIG. 8G). Additionally, activation of PAK1 and PAK2 following MRCKA depletion was observed in OVCAR4 cells by western blot suggesting that MRCKA may be part of a kinase feedback response on CDCl42/Rac signaling. Taken together, the proteomic analysis supports a role for MRCKA in promoting HGSOC through focal adhesion signaling, actin cytoskeleton remodeling, and cell cycle control.

### Example 5: MRCKA Essential for Focal Adhesion Signaling, Actin Remodeling and G1/S Transition in HGSOC Cells

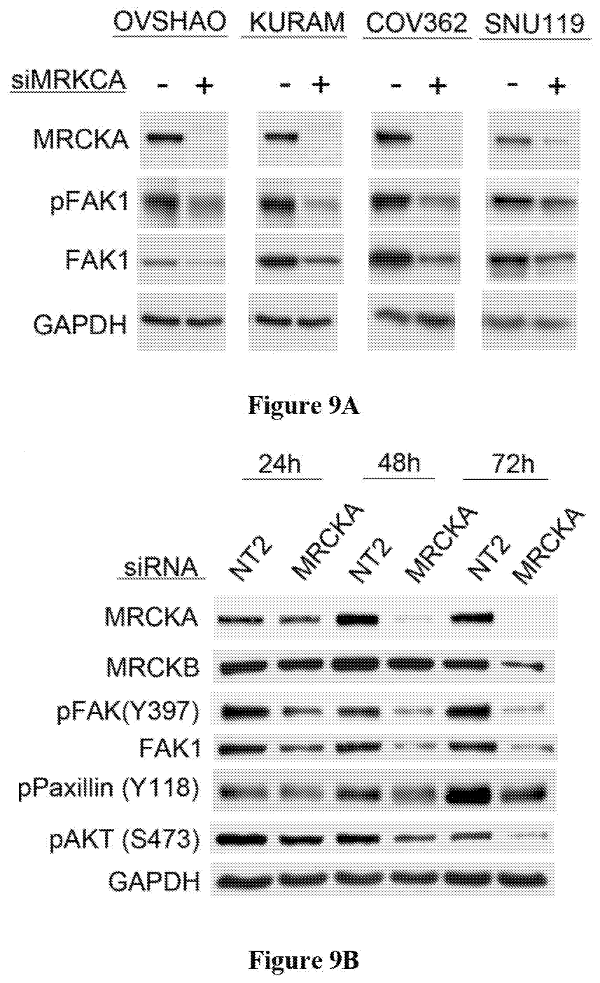

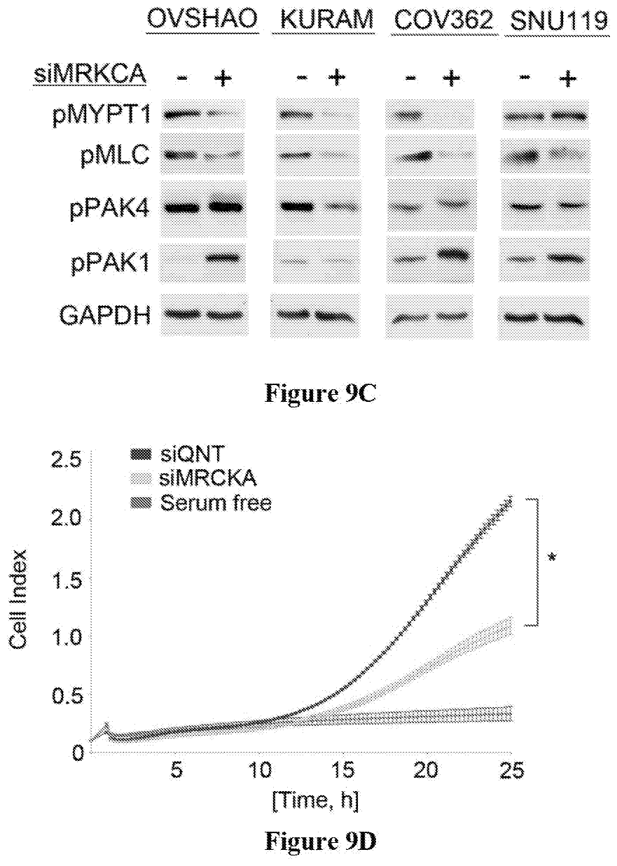

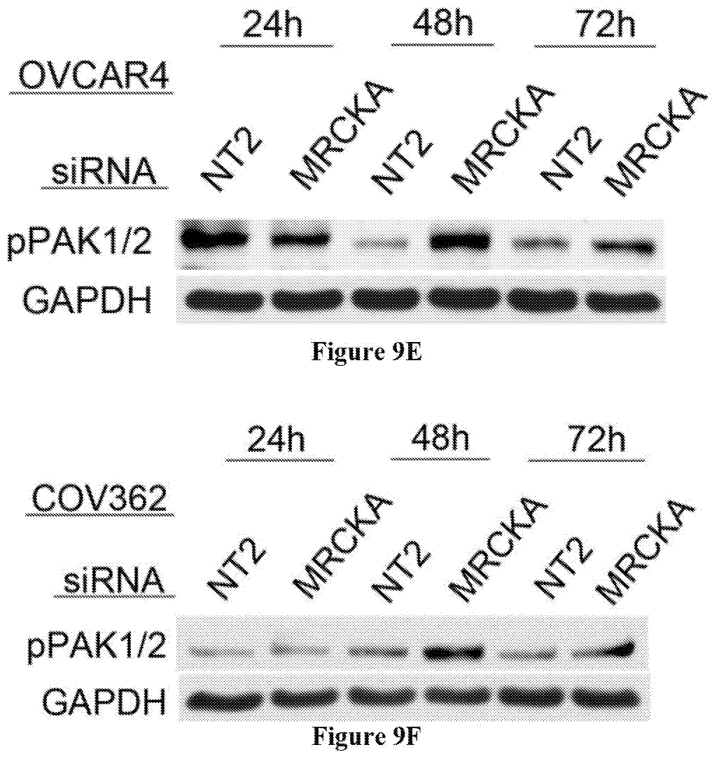

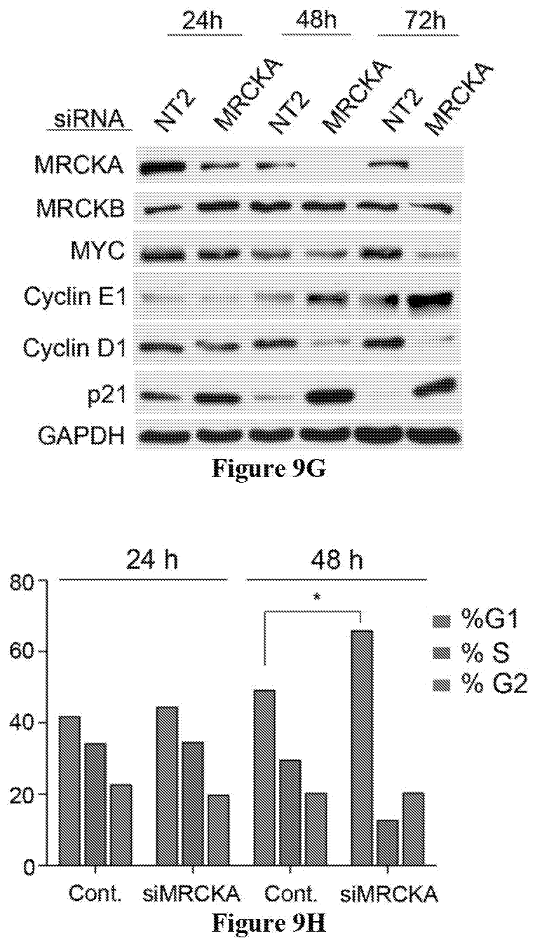

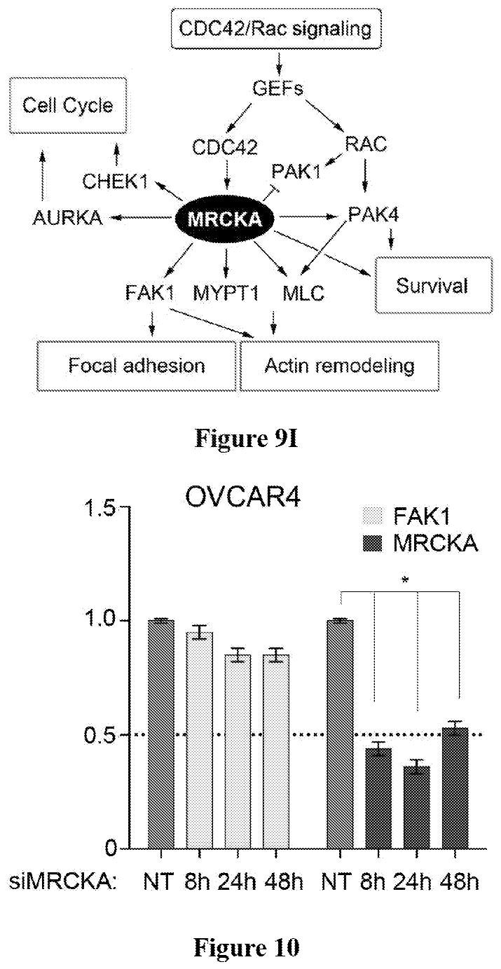

Targeting FAK1 in ovarian cancer has emerged as a promising therapeutic avenue due to its role in promoting ovarian cancer cell growth, survival and invasion (Stone et al., Cancer Biol. Ther., 2014, 15, 919-929; and Ward et al., Clin. Exp. Metastasis, 2013, 30, 579-594). Small molecule inhibition of FAK1 with PND-1186 or FAK1 knockdown reduced cell viability in HGSOC cells, confirming the dependence of HGSOC cells for FAK signaling (see, FIG. 5E). Interestingly, MRCKA knockdown reduced both FAK1 activating phosphorylation and total FAK1 protein levels in OVCAR4 cells, supporting a functional role for MRCKA in the regulation of FAK1 signaling in HGSOC cells. Moreover, reduced FAK1 activating phosphorylation and FAK1 protein levels were consistently observed across KURAMOCHI, COV362, SNU119 and OVSAHO HGSOC cells following MRCKA ablation (see, FIG. 9A). Time-dependent reduction in FAK1 mediated phosphorylation of substrate Paxillin at Y118 and downstream AKT signaling further demonstrated the negative consequence of MRCKA knockdown on focal adhesion signaling (see, FIG. 9B). Minimal reduction in FAK1 RNA levels was observed in OVCAR4 cells post MRCKA knockdown, signifying MRCKA protein depletion likely influences FAK1 at the protein level (see, FIG. 10).

Previous studies have implicated MRCKA as a downstream kinase of CDCl42, regulating actin remodeling, migration and invasion (Kale et al., Cancer Lett., 2015, 361, 185-196; and Prudnikova et al., Clin. Cancer Res., 2015, 21, 24). Consistent with these studies, reduced phosphorylation of MLC and MYPT1 was observed in MRCKA knockdown cells, and depletion of MRCKA inhibited cell motility of COV362 cells further establishing MRCKA as an essential kinase in cancer cell motility and invasion (see, FIGS. 9C and 9D). Reduced PAK4 activating phosphorylation and total protein levels were observed in KURAMOCHI and OVCAR4 cells following MRKCA knockdown, supporting a role for MRCKA downstream of Rac signaling in some HGSOC cell lines (see, FIGS. 9C and 7I). Elevated phosphorylation of PAK1 following MRCKA knockdown was observed in OVCAR4, OVSAHO, COV362 and SNU119 cells, signifying that loss of MRKCA protein from HGSOC cells results in compensatory activation of PAK1 signaling (see, FIGS. 9C, 9D, 9E, and 9F). These findings implicate MRKCA as a potential negative regulator of PAK1 signaling, where MRCKA may function in a negative feedback signaling cascade bridging CDCl42 and Rac signaling.

The proteomics studies demonstrated MRCKA knockdown results in reduced levels of cell cycle regulating kinases CDK4, AURKA and CHEK1, which are currently being targeted in clinical trials for HGSOC. To further characterize the consequence of MRCKA depletion on cell cycle control, cell cycle markers were profiled by western blot performance of FACS analysis. Knockdown of MRCKA reduced MYC, and Cyclin D1 protein levels and increased Cyclin E1 and p21 by 48 hours consistent with cell cycle arrest (see, FIG. 9G). FACS analysis of MRCKA knockdown cells showed an increase in percentage of cells in G1/S phase relative control siRNAs, demonstrating MRKCA knockdown induces a G1/S cell cycle arrest (see, FIG. 9H).

Collectively, the functional interrogation of MRCKA function in HGSOC cells demonstrates MRCKA as an essential kinase promoting FAK1-mediated focal adhesion signaling, MYPT1/MLC-mediated actin remodeling, G1/S cell cycle transition and cell survival. MRCKA may also be involved in the negative feedback regulation of PAK1/2 signaling in HGSOC cells, linking MRCKA to both CDCl42 and Rac cancer signaling (see, FIG. 9I).

### Example 6: Combined Blockade of MRCKA Enhances Current Therapies FDA-Approved or in Clinical Trials for HGSOC

The standard of care for HGSOC is aggressive debulking surgery followed by platinum and taxane-based chemotherapy, and recently PARP inhibitors have been FDA-approved for the treatment of recurrent HGSOC regardless of BRCA1/2 status (Lord et al., Nat. Med., 2013, 19, 1381; and Vaughan et al., Nat. Rev. Cancer, 2011, 11, 719-725). Although initially effective, patients ultimately develop resistance to carboplatin or PARP inhibitors for which there is no subsequent therapies. Furthermore, several targeted therapies are currently being explored in clinical trials for HGSOC including BET bromodomain inhibitors, as well as a variety of kinase inhibitors, including those targeting kinases WEE1 or CDK7.

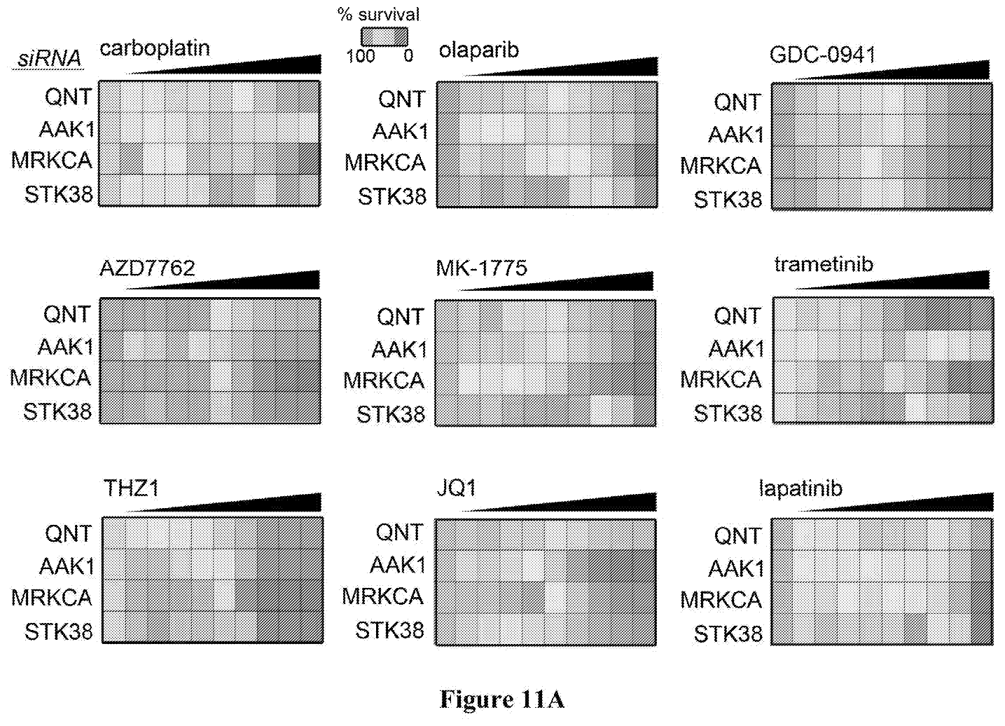

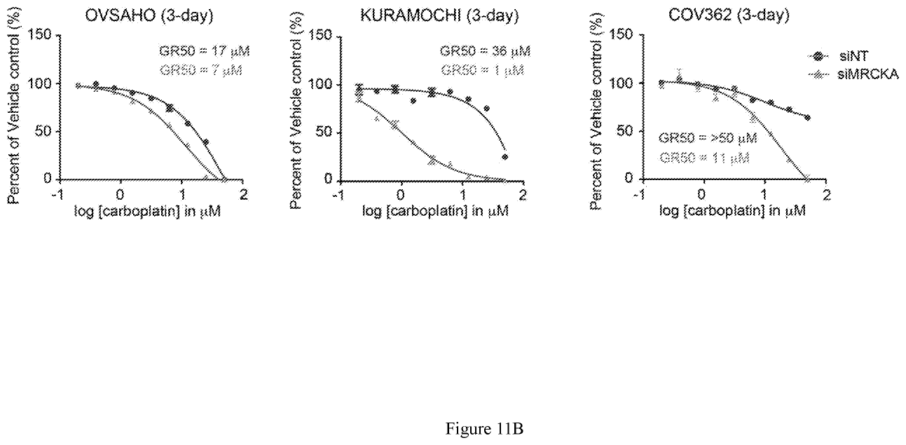

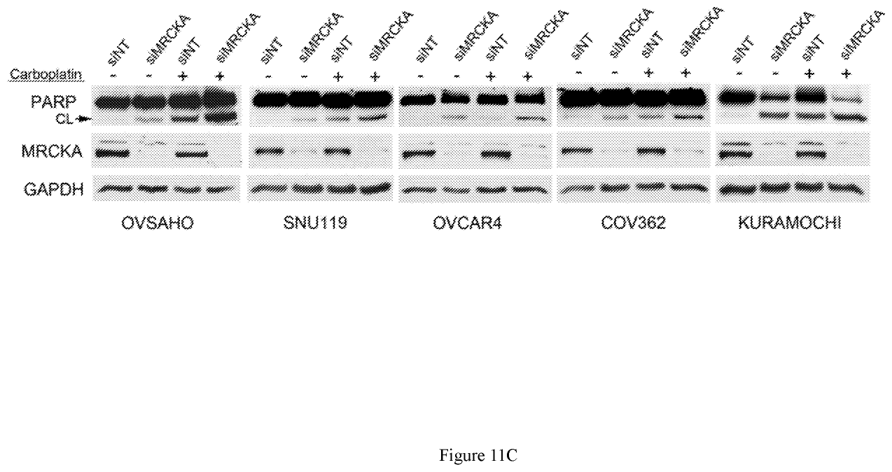

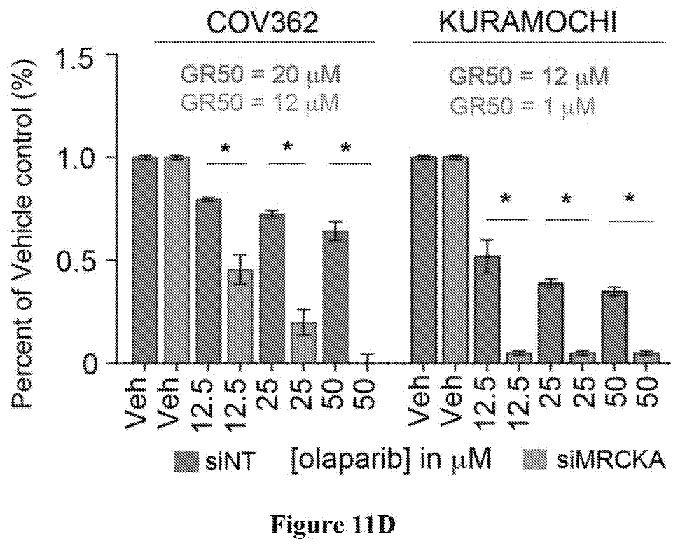

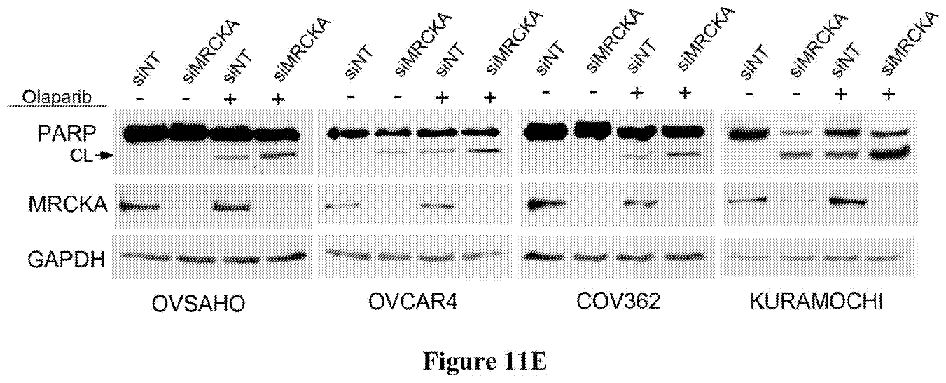

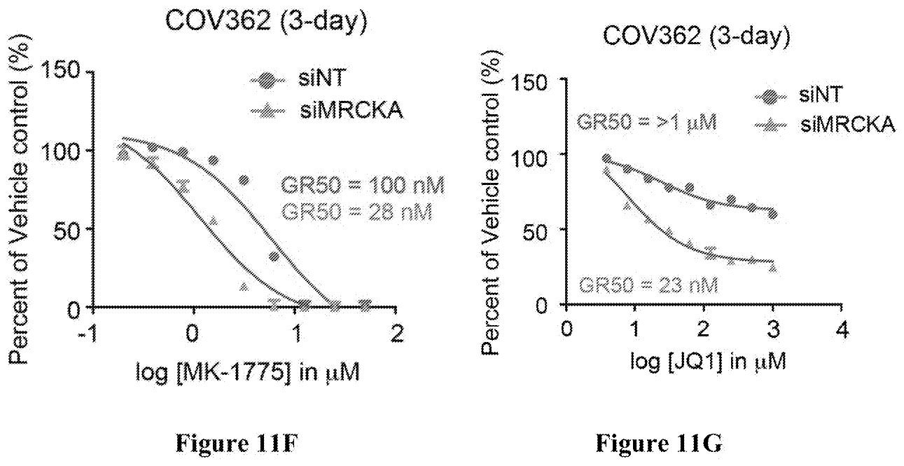

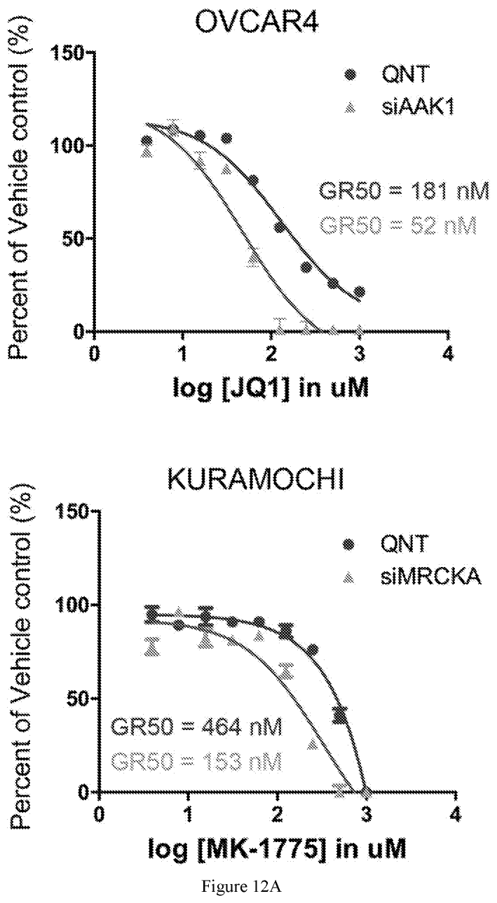

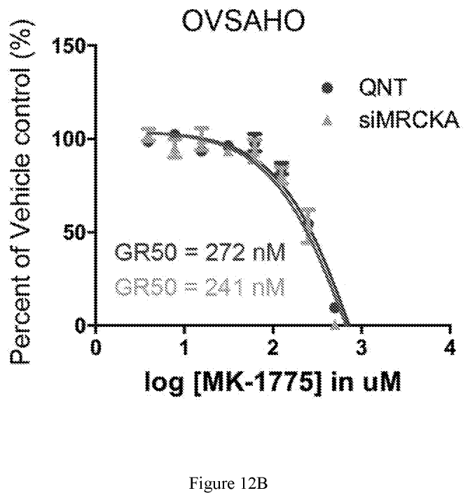

To explore if blocking dark kinases AAK1, STK38 or MRCKA activity in combination with current therapies improves drug efficacy, OVCAR4 cells depleted of AAK1, STK38 or MRCKA were treated with increasing doses of carboplatin, olaparib, JQ1, or a variety of kinase inhibitors in clinical trials for HGSOC. Knockdown of MRCKA enhanced growth inhibition of OVCAR4 cells in response to PI3Ki (GDC-0941), CDK7i (THZ1), BRD4i (JQ1), Wee1i (MK-1775), CHEK1 (AZD7762), PARPi (olaparib) or carboplatin relative to non-targeting control siRNA (see, FIG. 11A). Combined depletion of AAK1 and treatment with BET bromodomain inhibitor JQ1 enhanced growth inhibition, while knockdown of STK38 improved growth inhibition when combined with GDC-0941 or THZ1 (see, FIGS. 11A and 12A). Notably, MRCKA depletion enhanced the effect of carboplatin in additional HGSOC cell lines and induced apoptosis better than individual therapies (see, FIGS. 11B and 11C). Knockdown of MRCKA in combination with carboplatin inhibited cell growth and induced apoptosis in carboplatin-resistant COV362 cells, supporting a role for MRCKA in promoting survival in carboplatin-resistant cells (see, FIG. 11B). Additionally, knockdown of MRCKA improved the growth inhibitory and apoptotic response of olaparib treated HGSOC cells, demonstrating loss of MRCKA function represents a promising combination therapeutic strategy for both carboplatin and PARP inhibitor treatments (see, FIGS. 11D and 11E). Depletion of MRCKA also enhanced growth inhibition when combined with BET protein or WEE1 kinase inhibitors (see, FIGS. 11F, 11G, and 12B). Collectively, these findings demonstrated that interfering MRCKA function universally enhanced the efficacy of both chemo-based and targeted inhibitors in HGSOC, strongly supporting combination therapies that target MRCKA with additional agents for the treatment of HGSOC.

### Example 7: Evaluation of Emerging MRCKA Small Molecule Inhibitors in HGSOC Cells

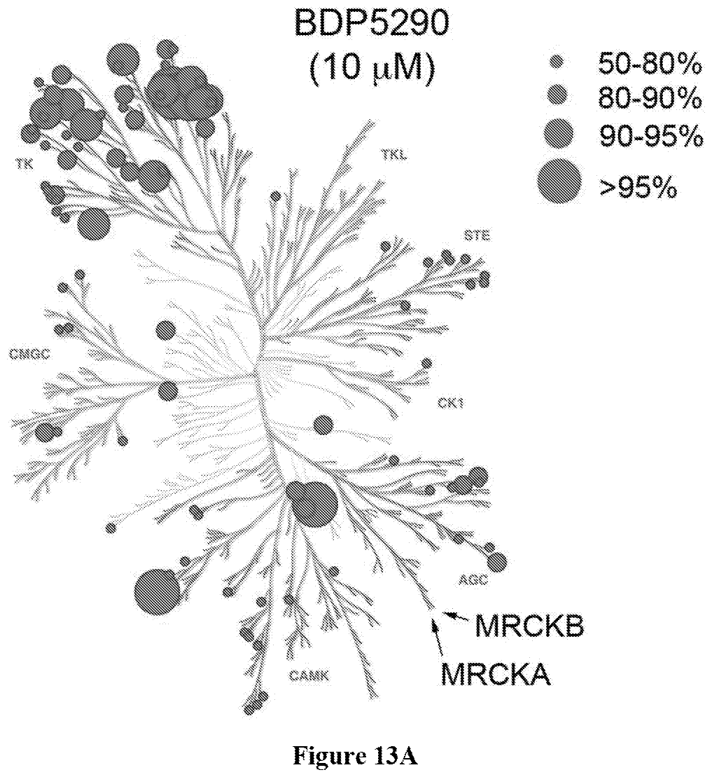

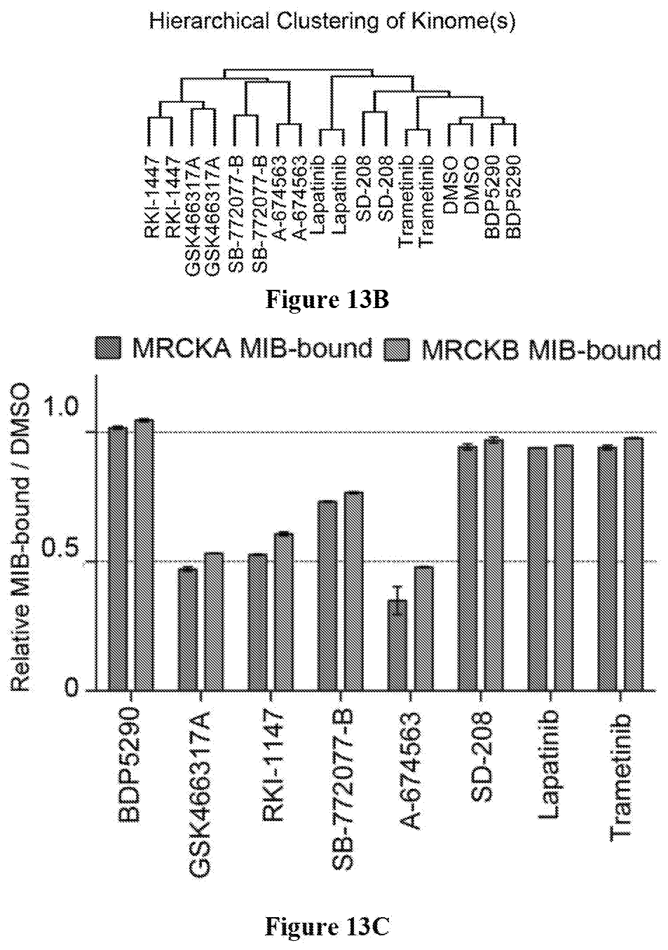

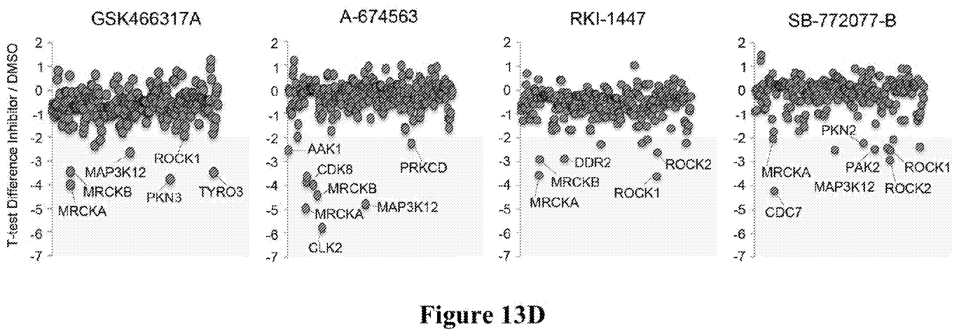

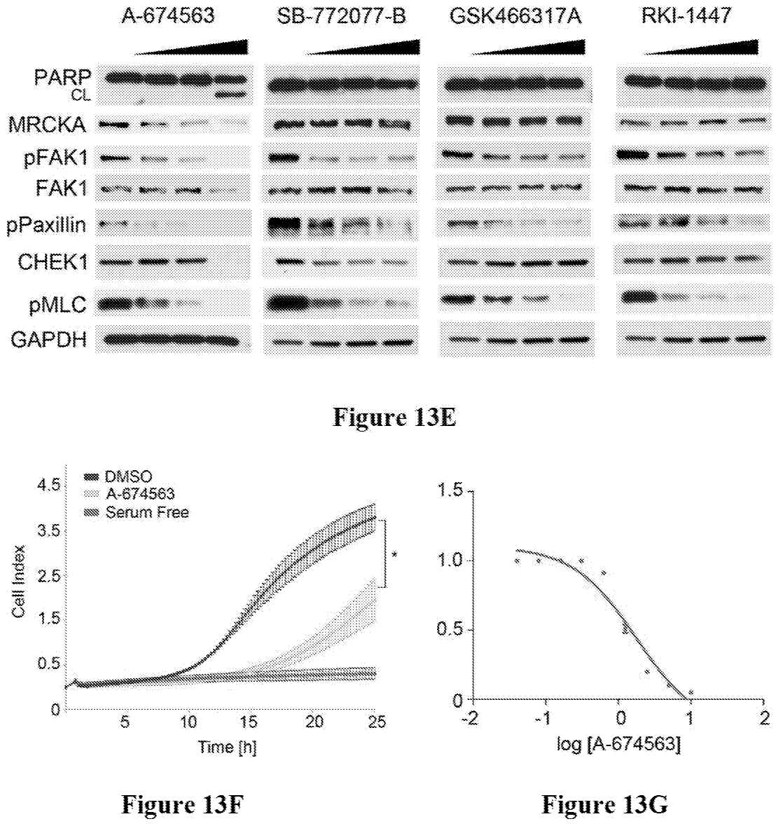

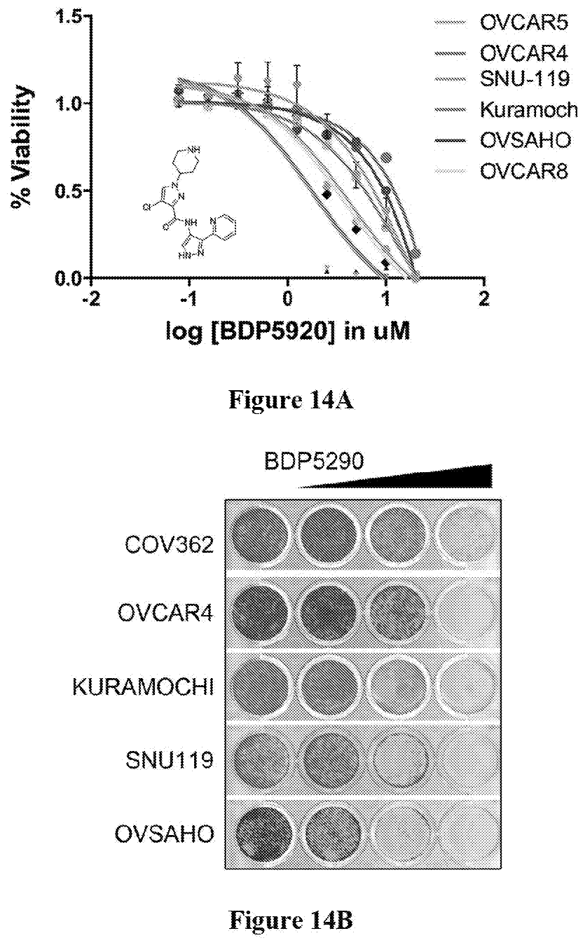

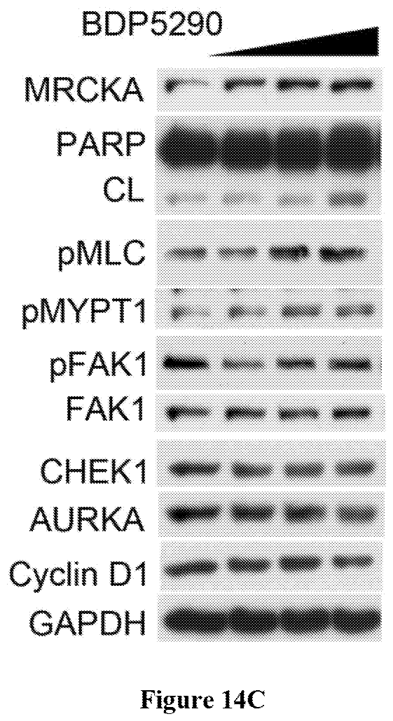

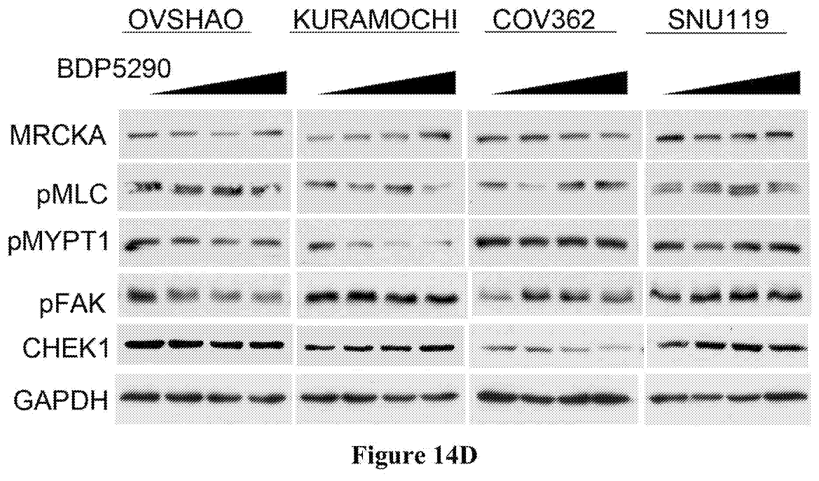

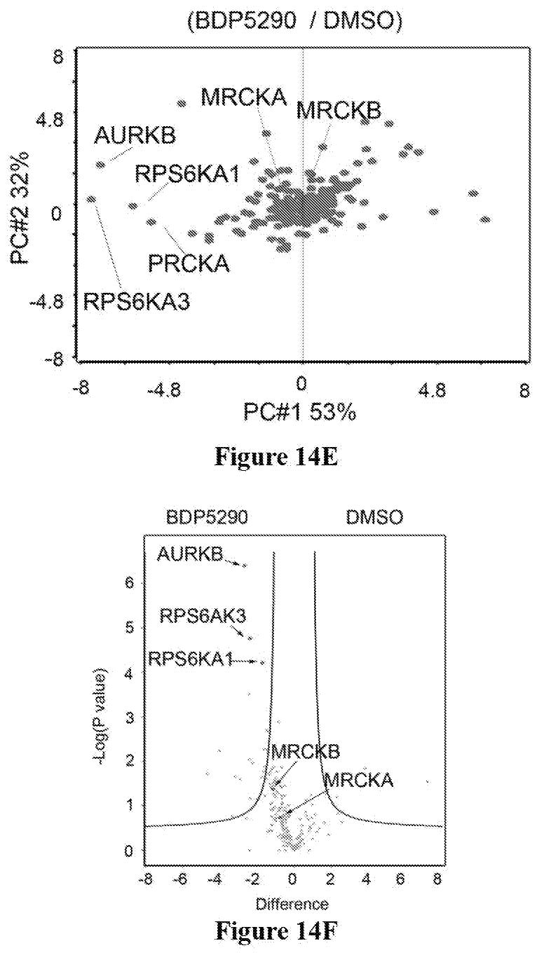

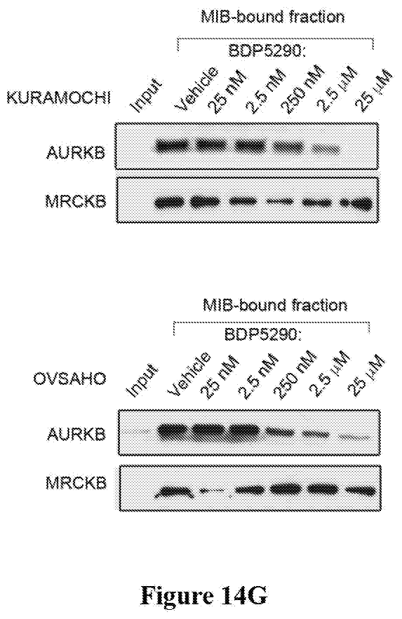

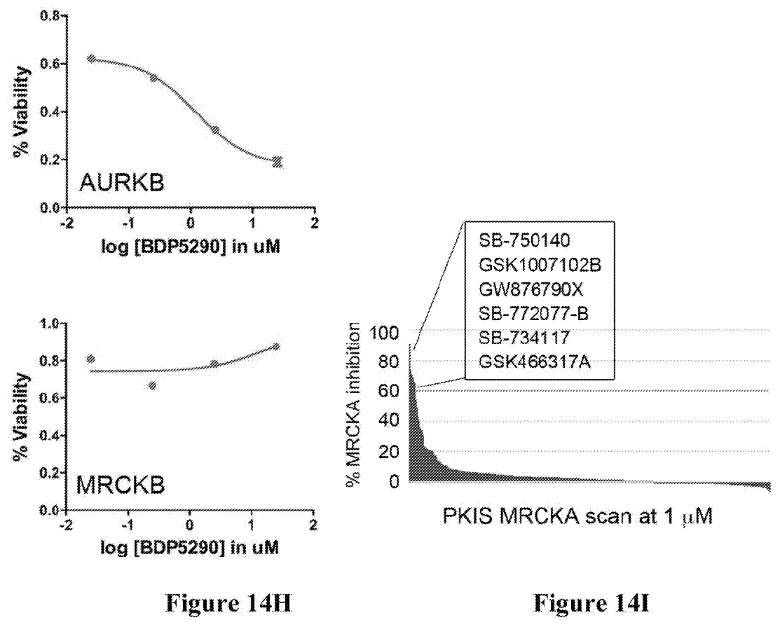

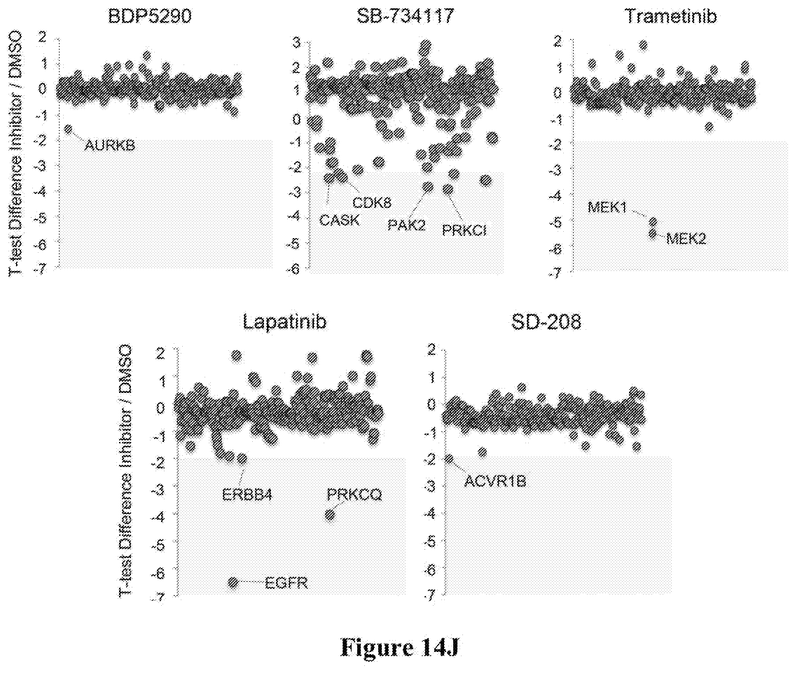

The RNAi-based studies revealed a role for the dark kinase MRCKA in HGSOC cell growth and survival through promoting a variety of oncogenic signaling, highlighting MRCKA as a promising kinase drug target in HGSOC. To determine the consequence of small molecule inhibition of MRCKA, HGSOC cells were treated with BDP5290, a commercially available MRCKA and MRCKB inhibitor with Ki values of 10 nM or 4 nM, respectively (Unbekandt et al., Cell Commun. Signal., 2014, 12, 54). Increasing doses of BDP5290 had minimal effects on the cell viability of HGSOC cells and did not reduce pMLC, pMYPT1, pFAK or total FAK1 or CHEK1 levels, inconsistent with MRCKA knockdown results (see, FIGS. 14A, 14B, 14C, and 14D). In vitro kinase assays (see, FIG. 13A) and proteomic analysis (see, FIGS. 14E, 14F, 14G, and 14H) of drug binders determined BDP5290 showed little activity against primary targets MRCKA or MRCKB in HGSOC cells at doses <5 μM but rather off-targets several kinases such as AURKB, prompting the search for more selective MRCKA inhibitors.

To identify MRCKA inhibitor probes, recent large-scale kinase inhibitor profiling studies were evaluated and several small molecules that inhibited MRCKA were identified, including ROCK1/2 inhibitors (RKI-1447 and SB-772077-B), a GRK2 inhibitor (GSK466317A), and an AKT inhibitor (A-674563) (Davis et al., Nat. Biotechnol., 2011, 29, 1046; Dhaliwal et al., J. Pharmacol. Exp. Ther., 2009, 330, 334; Elkins et al., Nat. Biotechnol., 2015, 34, 95; Homan et al., ACS Chem. Biol., 2015, 10, 310-319; Luo et al., Mol. Cancer Ther., 2005, 4, 977; and Patel et al., Cancer Res., 2012, 72, 5025) (see, FIG. 14I). MIB-based kinome profiling was performed on several of these molecules in HGSOC cells to determine kinome-wide inhibition profiles. Hierarchical clustering of MIB-binding ratios shows cells treated with RKI-1447, A-674563, SB-772077-B or GSK466317A exhibit similar response profiles that are distinct from DMSO, BDP5290, trametinib, lapatinib or SD-208 kinome signatures (see, FIG. 13B). Reduced MIB-binding of MRCKA and MRCKB was observed following a 4 hour treatment with 2 μM of RKI-1447, GSK466317A, A-674563 and to a lesser extent by SB-772077-B (see, FIG. 13C). A-674563 treatment exhibited the greatest reduction in MRCKA MIB-binding amongst inhibitors tested, while BDP5290 and positive controls trametinib (MEK1/2 inhibitor), SD-208 (ACVR1B/TGFRB inhibitor) or lapatinib (EGFR/HER2 inhibitor) had no effect on MRCKA/B MIB-binding. A reduction in MIB-binding of ROCK1 and ROCK2 was observed when cells were treated with RKI-1447, SB-772077-B, or GSK466317A (see, FIGS. 13D and 14J). A-674563 treatment did not influence ROCK1/2 MIB-binding but inhibited several kinases including AAK1, CDK8, PRCKD, MAP3K12 and CLK2 MIB-binding.

Treatment of HGSOC cells with increasing doses of GSK466317A, RKI-1174, or SB-772077-B reduced FAK1, MLC and Paxillin phosphorylation similar to what was observed following MRCKA knockdown (see, FIG. 13E). However, as ROCK1/2 have established roles in MLC and FAK signaling, the contribution of blocking ROCK1/2 function in addition to MRCK kinases with these inhibitors cannot be excluded. Notably, A-674563, which did not target ROCK1/2 in MIB-assays, inhibited FAK1, Paxillin and MLC phosphorylation, uniquely reduced FAK1 and CHEK1 protein levels, as well as induced apoptosis. Moreover, treatment of cells with A-674563 reduced cell migration and inhibited cell viability consistent with genetic ablation of MRCKA (see, FIGS. 13F and 13G). Collectively, GSK466317A, RKI-1174, and SB-772077-B target several kinases in addition to MRCKA, including ROCK1/2, making it difficult to resolve MRCKA-specific functions from that of ROCK kinases. The small molecule A-674563 targets several kinases including MRCKA in HGSOC cells but exhibited the most molecular overlap with MRCKA knockdown amongst the small molecules evaluated. Although A-674563 is not a selective MRCKA inhibitor, it represents a promising inhibitor probe to explore MRKCA function that can be used to facilitate the design of more selective MRCKA inhibitors.

The protein kinome represents one of the most promising and actionable classes of drug targets for the treatment of cancer; yet, the majority of the kinome remains untargeted for drug therapy, with many kinases having no established oncogenic function (Fedorov et al., Nat. Rev. Cancer, 2016, 16, 83-98; and Klaeger et al., Science, 2017, 358, eaan4368). To probe the dark kinome for novel kinase vulnerabilities and anti-cancer targets, a large-scale proteomic analysis of the ovarian cancer kinome in HGSOC patient tumors, PDX tumors and cell lines was performed. Profiling of HGSOC tumor and cell kinome(s) revealed a network of previously established kinase drivers, including CK2, BRAF, CDK7, MAP2K1, ERK1, JAK1, PAK4, PTK2, CHEK1, WEE1 and AKT1 with majority currently in clinical trials for HGSOC. Furthermore, kinome profiling of HGSOC using Q-MIBs revealed several dark kinases that shared MIB-binding ratios across patient and PDX tumors that were distinct from adjacent normal ovary tissue including AAK1, STK38, NUAK1, STK35 and MRCKA. Interrogation of growth and survival functions of these understudied kinases in validated HGSOC cells nominated MRCKA as a promising anti-cancer target in HGSOC. siRNA-mediated knockdown of MRKCA uniformly reduced cell viability across HGSOC cells and induced apoptosis in several, establishing MRCKA as an essential kinase in HGSOC.

The kinome, phosphoproteomics and single-run proteome analysis of MRCKA knockdown in HGSOC cells revealed a role for MRCKA in promoting focal adhesion, actin remodeling, cell cycle progression and cell survival. These findings are consistent with previous reports indicating MRCKA functions in cell migration and invasion in breast and lung cancer cells, acting downstream of CDCl42 regulating actin remodeling through phosphorylation of MYPT1 and MLC (Kale et al., Cancer Lett., 2015, 361, 185-196; Prudnikova et al., Clin. Cancer Res., 2015, 21, 24; Unbekandt and Olson, J. Mol. Med. (Berl), 2014, 92, 217-225; Zhao et al., Small GTPases, 2015, 6, 81-8). Interestingly, genetic ablation of MRCKA also led to activation of PAK1 in HGSOC cells, suggesting a potential feedback mechanism linked to MRCKA protein loss in HGSOC cells. As both MRCKA and PAK1 compete for CDCl42 binding in cells, the reduction in MRCKA levels may allow more binding of PAK1 to CDCl42 resulting in PAK1 activation. As PAK1 is an established oncogenic kinase in ovarian cancer (Prudnikova et al., Oncogene, 2016, 35, 2178-2185), combination therapies blocking PAK1 feedback activation may be required to achieve durable responses when targeting MRCKA in HGSOC.

In addition to a role in actin remodeling, evidence is provided herein that MRCKA protein is essential for FAK1 activity and protein stability, as knockdown of MRCKA reduced both activating FAK1 phosphorylation and total protein levels independent of RNA changes. FAK1 is a highly tractable anti-cancer target in HGSOC (Ward et al., Clin. Exp. Metastasis, 2013, 30, 579-594) and the studies herein suggest that inhibition of MRCKA may provide an additional therapeutic strategy to interfere with FAK1 activity in HGSOC.

Knockdown of MRCKA sensitized HGSOC cells to carboplatin, PARP inhibitors, BET bromodomain inhibitors, as well as several kinase inhibitors currently in clinical trials for HGSOC. The overall effectiveness of MRCKA knockdown in enhancing the efficacy of a variety of clinically relevant drug therapies strongly supports MRKCA as promising kinase inhibitor target for the treatment of HGSOC. In the studies described herein, the commercially available MRCKA inhibitor BDP5290 exhibited minimal potency at doses <5 μM in HGSOC cells. A-674563 has been identified herein as a MRKCA inhibitor, though not selective, A-674563 reproduced the effects observed with MRCKA knockdown in HGSOC cells.

### Example 8: Experimental Procedures

### Cell Lines

Cell lines were verified by IDEXX laboratories. OVCAR4, KURAMOCHI, and OVSAHO cell lines were maintained in RPMI-1640 supplemented with 10% FBS, 100 U/ml Penicillin-Streptomycin and 2 mM GlutaMAX. SNU-119 cells were maintained in RPMI-1640 supplemented with 10% FBS, 100 U/ml Penicillin-Streptomycin, 2 mM GlutaMAX and 25 mM HEPES. COV362 cells were maintained in DMEM supplemented with 10% FBS, 100 U/ml Penicillin-Streptomycin and 2 mM GlutaMAX. Heavy kinome standard cell lines (MOLT4, UACC257, ACHN, Colo205, and PC-3) were grown for seven doublings in arginine- and lysine-depleted media supplemented with heavy isotope labeled [¹³C₆,¹⁵N₄]arginine (Arg¹⁰) (84 mg/L) and [¹³C₆]lysine (Lys⁸) (48 mg/L) (Sigma), and unlabeled leucine (50 mg/L) (ThermoFisher Scientific). All cells were kept at 37° C. in a 5% CO₂ incubator.

### Patient Samples

All patient samples were collected at Fox Chase Cancer Center, with approval from the Institutional Review Board. Consent and authorization was obtained at the time of specimen donation to the FCCC bio repository.

### Frozen Tumor Samples

Ovarian tumors from eleven chemo-naive patients with stage III or IV HGSOC were collected under an approved IRB (16-9031 and 14-809) protocol at Fox Chase Cancer Center during the primary debulking surgery. Tumors were snap frozen and stored at −80° C. until sample processing for MIB-MS analysis. Serous histology was confirmed by FCCC a gynecology pathologist at FCCC. Normal adjacent ovary tissue was collected during primary debulking surgery and snap frozen and stored at −80° C. Tumors isolated from HGSOC patient-derived xenografts (PDX) models were snap frozen and stored at −80° C. until sample processing for MIB-MS analysis.

### Compounds

A-674563, AZD7762, Carboplatin, Cediranib, GDC0941, GSK1120212 (Trametinib), GSK1904529A, Lapatinib, LY3009120, MK-1775, MK-2206, MLN-8237 (Alisertib), Olaparib, Palbociclib, PND-1186, and RKI-1447 were purchased from Selleckchem. JQ1 and THZ1 were purchased from ApexBio. BDP5290, SD-208, Vx11e, and Vx970 (VE-822) were purchased from Glixx Labs, Tocris, Chemietek, and MedKoo respectively. SB-772077-B, GSK466317A, and SB-734117 were obtained through the Structural Genomics Consortium. For the compounds utilized in MIB synthesis, Purvalanol B was purchased from Abcam. PP58 (Klutchko et al., J. Med. Chem., 1998, 41, 3276-3292) and VI16832 (Daub et al., Mol. Cell., 2008, 31, 438-448) were custom synthesized according to previously described methods by The Center for Combinatorial Chemistry and Drug Discovery, Jilin University, P.R. China. CTx-0294885 (Zhang et al., J. Proteome Res., 2013, 12, 3104-3116) was purchased from Medkoo. Conjugation of inhibitors to beads was performed by carbodiimide coupling to ECH Sepharose 4B (CTx-0294885, VI16832 and PP58) or EAH Sepharose 4B (purvalanol B) (GE Healthcare).

### Western Blotting

Samples were harvested in MIB lysis buffer, subjected to SDS-PAGE chromatography and transferred to PVDF membranes before western blotting with primary antibodies. For pMYPT and pMLC blots, cells were harvested in a buffer containing 1% (w/v) SDS, 50 mM Tris pH 7.5 supplemented with protease and phosphatase inhibitors buffer and were passed through QIAshredder columns (Qiagen, 79654), (Unbekandt et al., Cell Commun. Signal., 2014, 12, 54). For the list of primary antibodies used, see Table 4. Secondary HRP-anti-rabbit and HRP-anti-mouse were obtained from ThermoFisher Scientific. SuperSignal West Pico and Femto Chemiluminescent Substrates (Thermo) were used to visualize blots.

TABLE 4
Primary antibodies used in the study

| Antibody | Manufacturer | Cat # |
|---|---|---|
| AAK1 | AbCam | ab134971 |
| AAK1 | SCBT | sc-134242 |
| AKT1 | CST | 2967 |
| AKT2 | CST | 2964 |
| AKT3 | CST | 4059 |
| Aurora A | CST | 14475 |
| BRAF | CST | 9433 |
| CDK7 | CST | 2916 |
| CHEK1 | CST | 2360 |
| Cyclin E1 | SCBT | sc-247 |
| CyclinD1 | CST | 2978 |
| DDR1 | CST | 5583 |
| EIF2AK2 (PKR) | CST | 12297 |
| ERK ½ | CST | 4696 |
| FAK1 | CST | 3285 |
| FGFR2 (Bek) | SCBT | sc-122 |
| GAPDH | CST | 2118 |
| HER2/ErbB2 | CST | 2165 |
| HER4/ErbB4 | CST | 4795 |
| JAK1 | SCBT | sc-277 |
| MEK1 | SCBT | sc-219 |
| MRCKa | SCBT | sc-374568 |
| MRCKa | AbCam | ab96659 |
| MRCKb | SCBT | sc-390127 |
| Myc | CST | 5065 |
| NUAK1 (ARK5) | CST | 4458 |
| p21 | SCBT | sc-6246 |
| p70S6K T389 | CST | 9234 |
| PAK4 | CST | 3242 |
| pAKT S473 | CST | 4060 |
| pAKT T308 | CST | 13038 |
| pAP2M1 T156 | CST | 3843 |
| PARP | CST | 9542 |
| pBRAF S445 | CST | 2696 |
| pDDR1 Y513 | CST | 14531 |
| PDGFRβ | CST | 4564 |
| pERK T202/204 | CST | 4370 |
| pFAK Y397 | CST | 8556 |
| pFGFR Y653/54 | CST | 3471 |
| pHER2/ErbB2 Y1221/23 | CST | 2249 |
| pHER4/ErbB4 Y984 | CST | 3790 |
| pJak1 Y1022/1023 | CST | 3331 |
| pMEK S217/221 | CST | 9154 |
| pMLC | CST | 3674 |
| pMyc S62 | CST | 13748 |
| pMYPT | CST | 5163 |
| pPAK1 S144/PAK2 S141 | CST | 2606 |
| pPAK4 S474/PAK5 S602/PAK6 | CST | 3241 |
| S560 | | |
| pPaxillin Y118 | CST | 2541 |
| PRKCQ (PKC Theta) | CST | 13643 |
| pSrc Family Y416 | CST | 6943 |
| STK38 | AbCam | ab-37974 |
| WEE1 | CST | 4936 |
| Supp | | |
| pALK | AbCam | ab73996 |
| ALK | AbCam | ab140534 |
| AuroraB | AbCam | ab2254 |

### Growth Assays

For short-term growth assays, 3000-5000 cells were plated per well in 96-well plates and allowed to adhere and equilibrate overnight. Drug was added the following morning and after 120 hours of drug treatment, cell viability was assessed using the CellTiter-Glo Luminescent cell viability assay according to manufacturer (Promega). Students t tests were performed for statistical analyses and p values ≤0.05 were considered significant. For colony formation assays, cells were plated in 24-well dishes (3000-5000 cells per well) and incubated overnight before continuous drug treatment for 14 days, with drug and media replaced twice weekly. At the end of treatment, cells were rinsed with PBS and fixed with chilled methanol for 10 minutes at −20° C. Methanol was removed by aspiration, and cells were stained with 0.5% crystal violet in 20% methanol for 20 minutes at room temperature, washed with distilled water, and scanned.

qRT-PCR

GeneJET RNA purification kit (Thermo Scientific) was used to isolate RNA from cells according to manufacturer's instructions. qRT-PCR on diluted cDNA was performed with inventoried TaqManR Gene Expression Assays on the Applied Biosystems 7500 Fast Real-Time PCR System. The TaqMan Gene Expression Assay probes (ThermoFisher Scientific) used to assess changes in gene expression include CDCl42BPA (Assay ID: Hs00177522_m1), PTK2 (Assay ID: Hs00178587_m1), AAK1 (Assay ID: Hs00208618_m1), STK35 (Assay ID: Hs00934629_g1), and STK38 (Assay ID: Hs00179367_m1).

### RNAi Knockdown Studies

siRNA transfections were performed using 25 nM siRNA duplex and the reverse transfection protocol. 3000-5000 cells per well were added to 96 well plates with media containing the siRNA and transfection reagent (Lipofectamine RNAiMax) according to the manufacturer's instructions. In experiments where inhibitors were used, the inhibitor was added at the time of transfection. Cells were allowed to grow for 72-120 hours post-transfection prior to CellTiter Glo (Promega) analysis. Two-to-three independent experiments were performed with each cell line and siRNA. Students t tests were performed for statistical analyses and p values ≤0.05 were considered significant. For western blot studies, the same procedure was performed with volumes and cell numbers proportionally scaled to a 60 mm or 10 cm dish, and cells were collected 24-72 hours post-transfection. siRNA product numbers and manufacturers are listed in Table 5.

siRNAs used in the study

| siRNA | | |
|---|---|---|
| GE Dharmacon | Type | Cat. # |
| AAK1 | siGENOME/SMARTpool | M-005300-01 |
| AKT1 | siGENOME/SMARTpool | M-003000-03 |
| AKT2 | siGENOME/SMARTpool | M-003001-02 |
| AKT3 | siGENOME/SMARTpool | M-003002-02 |
| ATM | siGENOME/SMARTpool | M-003201-04 |
| ATR | siGENOME/SMARTpool | M-003202-05 |
| AURKA | siGENOME/SMARTpool | M-003545-10 |
| BRAF | siGENOME/SMARTpool | M-003460-03 |
| CDC42BPA | siGENOME/SMARTpool | M-003814-04 |
| CDC42BPA #1 | siGENOME/Individual siRNA | D-003814-04 |
| CDC42BPA #2 | siGENOME/Individual siRNA | D-003814-08 |
| CDK7 | siGENOME/SMARTpool | M-003241-02 |
| CHEK1 | siGENOME/SMARTpool | M-003255-04 |
| CHEK2 | siGENOME/SMARTpool | M-003256-06 |
| CLK2 | siGENOME/SMARTpool | M-004801-02 |
| DDR1 | siGENOME/SMARTpool | M-003111-04 |
| EGFR | siGENOME/SMARTpool | M-003114-03 |
| EIF2AK2 | siGENOME/SMARTpool | M-003527-00 |
| ERBB3 | siGENOME/SMARTpool | M-003127-03 |
| ERBB4 | siGENOME/SMARTpool | M-003128-03 |
| FGFR2 | siGENOME/SMARTpool | M-003132-04 |
| IGF1R | siGENOME/SMARTpool | M-003012-05 |
| JAK1 | siGENOME/SMARTpool | M-003145-02 |
| MAP2K1 | siGENOME/SMARTpool | M-003571-01 |
| MAP2K2 | siGENOME/SMARTpool | M-003573-03 |
| MARK2 | siGENOME/SMARTpool | M-004260-02 |
| MTOR | siGENOME/SMARTpool | M-003008-03 |
| NUAK1 | siGENOME/SMARTpool | M-004931-00 |
| PAK4 | siGENOME/SMARTpool | M-003615-03 |
| PIK3CA | siGENOME/SMARTpool | M-003018-03 |
| PIP4K2C | siGENOME/SMARTpool | M-004535-00 |
| PRKCQ | siGENOME/SMARTpool | M-003525-01 |
| PTK2 | siGENOME/SMARTpool | M-003164-02 |
| PTK2B | siGENOME/SMARTpool | M-003165-03 |
| RPS6KB1 | siGENOME/SMARTpool | M-003616-03 |
| STK35 | siGENOME/SMARTpool | M-005384-01 |
| STK38 | siGENOME/SMARTpool | M-004674-01 |
| UBB | siGENOME/SMARTpool | M-013382-01 |
| WEE1 | siGENOME/SMARTpool | M-005050-02 |
| Non-Targeting | siGENOME/Control | D-001206-14 |
| siRNA Pool #2 | | |
| Qiagen | | |
| AllStars Negative | Control | SI03650318 |
| Control siRNA | | |

### Migration Assay

Cell migration assays were performed using the xCELLigence RTCA DP Instrument (ACEA Biosciences) in a CIM-Plate 16 (ACEA Biosciences). Complete medium was added to the lower chambers of the CIM-Plate (or serum free medium for negative control). In the upper chambers, 30 μL serum free medium was added to each well and the CIM-Plate was placed in the instrument to equilibrate for 1 hr at 37° C. Following this equilibration step, a background measurement was taken. The CIM-Plate was removed from the instrument, and COV362 cells were added to the upper chambers at 50,000 cells per well in serum free medium. The CIM-Plate was left at 25° C. for 30 minutes to allow the cells to settle at the bottom of the well. The plate was then returned to the instrument, and cell index reads were taken once every 15 minutes for 24 hours. For the siRNA experiment, the cells were transfected 24 hours prior to the start of the assay. For the A-674563 experiment, cells were pretreated with 1 μM inhibitor or DMSO 24 hours prior, and both upper and lower chambers contained 1 μM inhibitor or DMSO.

### BDP5290 Selectivity Profiling Assay

OVSAHO and KURAMOCHI lysates (5 mg of total protein each) were preincubated with 0 (DMSO control), 2.5 nM, 25 nM, 250 nM, 2.5 μM or 25 μM BDP5290 on an end-over-end shaker for 30 minutes at 4° C. Lysates were then incubated with MIBs for 45 minutes at 4° C. (Medard et al., J. Proteome Res., 2015, 14, 1574-1586) and added to gravity flow columns. The MIB preparation protocol was subsequently followed excluding the reduction and alkylation steps. Following methanol/chloroform precipitation, the precipitated pellet was resuspended in 20 μL complete MIBs lysis buffer and 4 μL 6×LSB was added to each sample. Samples were boiled at 100° C. for 5 minutes and subjected to western blot analysis. For the selectivity assay analyzed via LC-MS/MS, 5 mg OVSAHO lysates were preincubated with the same drug concentrations and incubated with MIBs, and the MIB preparation protocol was followed.

### MIBs Preparation and Chromatography

Experiments using MIB/MS were performed as previously described (Duncan et al., Cell, 2012, 149, 307-321; and Kurimchak et al., Cell Reports, 2016, 16, 1273-1286). Briefly, cells or tumors were lysed on ice in buffer containing 50 mM HEPES (pH 7.5), 0.5% Triton X-100, 150 mM NaCl, 1 mM EDTA, 1 mM EGTA, 10 mM sodium fluoride, 2.5 mM sodium orthovanadate, 1× protease inhibitor cocktail (Roche), and 1% each of phosphatase inhibitor cocktails 2 and 3 (Sigma). Particulate was removed by centrifugation of lysates at 13,000 rpm for 10 minutes at 4° C. and filtration through 0.2 μm syringe filters. Protein concentrations were determined by BCA analysis (Thermo). To compare kinome signatures within and across such large sample sizes, a super-SILAC method was designed (Neubert et al., Nat. Meth., 2010, 7, 361-362) consisting of a cocktail of cancer cell lines that encompasses the activity of the “cancer kinome” to be used as a control for individual samples. Five cancer cell lines were selected from the NCI-60 panel (UACC257, MOLT4, COL0205, ACHN and PC3) that differed in their origin, gene expression patterns and mutation status. The cancer cell lines were labeled using SILAC and mixed equally providing a diverse SILAC ([¹³C₆,¹⁵N₄] arginine (Arg¹⁰) and [¹³C₆] lysine (Lys⁸)) heavy kinome reference standard (HKS) to be used repeatedly in kinome profiling assays. An equal amount of the HKS was added to the non-labeled sample (cell, tumor or normal tissue) providing a quantitative strategy to compare kinome activity/levels across samples, amongst treatments and, importantly, across time. Endogenous kinases were isolated by flowing lysates over kinase inhibitor-conjugated Sepharose beads (purvalanol B, VI16832, PP58 and CTx-0294885 beads were used) in 10 ml gravity-flow columns. After 2×10 ml column washes in high-salt buffer and 1×10 ml wash in low-salt buffer (containing 50 mM HEPES (pH 7.5), 0.5% Triton X-100, 1 mM EDTA, 1 mM EGTA, and 10 mM sodium fluoride, and 1M NaCl or 150 mM NaCl, respectively), retained kinases were eluted from the column by boiling in 2×500 μL of 0.5% SDS, 0.1 M TrisHCl (pH 6.8), and 1% 2-mercaptoethanol. Eluted peptides were reduced by incubation with 5 mM DTT at 60° C. for 25 minutes, alkylated with 20 mM iodoacetamide at room temperature for 30 minutes in the dark and alkylation was quenched with DTT. Samples were concentrated to approximately 100 μL with Millipore 10 kD cutoff spin concentrators. Detergent was removed by chloroform/methanol extraction, and the protein pellet was resuspended in 100 mM ammonium bicarbonate and digested with sequencing-grade modified trypsin (Promega) overnight at 37° C. SILAC-labeled and non-labeled kinase peptides were cleaned with PepClean C18 spin columns (Thermo), and subsequent LC/MS analysis was performed.

### Mass Spectrometry and Spectra Analysis

Proteolytic peptides were resuspended in 0.1% formic acid and separated with a Thermo RSLC Ultimate 3000 on a Thermo Easy-Spray C18 PepMap 75 μm×50 cm C-18 2 μm column with a 240 minute gradient of 4-25% acetonitrile with 0.1% formic acid at 300 nL/min and 50° C. Eluted peptides were analyzed by a Thermo Q Exactive plus mass spectrometer utilizing a top 15 methodology in which the 15 most intense peptide precursor ions were subjected to fragmentation. The AGC for MS1 was set to 3×106 with a max injection time of 120 ms, and the AGC for MS2 ions was set to 1×105 with a max injection time of 150 ms and the dynamic exclusion was set to 90 s. Protein identification was performed by searching MS/MS data against the swiss-prot human protein database downloaded on Jul. 26, 2017 using andromeda 1.5.6.0 built in MaxQuant 1.6.1.0 and sequestHT built in Proteome Discoverer 2.2. The search was set up for full tryptic peptides with a maximum of two missed cleavage sites. Acetylation of protein N-terminus and oxidized methionine were included as variable modifications and carbamidomethylation of cysteine was set as fixed modification. The precursor mass tolerance threshold was set 10 ppm for and maximum fragment mass error was 0.02 Da. SILAC quantification was performed using MaxQuant by choosing multiplicity as 2 in group-specific parameters and Arg10 and Lys8 as heavy labels. Imputation of missing values was performed as previously described (Deeb et al., Mol. Cell. Proteomics, 2012, 11, 77-89), where in the super-SILAC data, a width of 0.3 and the downshift of 0.5 was employed. Label free quantification was carried out using MaxQuant and proteome discoverer 2.2 software. The significance threshold of the ion score was calculated based on a false discovery rate of <1%. Most of the parameters were used as default until unless specified.

### Statistical Analysis and Visualization of MIB/MS Signatures

For Q-MIBs signatures, at least 2 biological MIB/MS experiments run in technical replicates on the Q Exactive was performed. Q Exactive data was analyzed as follows: for a total of p unique kinases, the pooled protein ratio and p-value across the replicates were computed. For each replicate, kinases that exhibit statistically significant changes in expression were identified based on stepup-adjusted p-values at FDR of 0.05 to account for multiple comparisons. Visualization of MIBs-kinome signatures using PCA plots, Box-and-whiskers plots or Log2 ratio heat maps were generated using Partek Genomics Suite and Perseus Software v 1.5.2.6. R-squared regression analysis MIB/MS runs were performed on Perseus Software v 1.5.2.6. Only kinases that were captured in independent experiments were considered for statistical analysis, and resulting statistically significant kinases were combined to generate signatures.

### Phosphoproteomics and Single-Run Proteome Analysis

To define kinase and kinase substrate phosphorylation at baseline or in response to kinase knockdown with performed global phosphoproteomics analysis, as previously described (Thingholm et al., Nat. Prot., 2006, 1, 1929-1935). Briefly, using methanol/chloroform protein precipitation methods, a 4 mg lysate (2 mg sample, 2 mg of HKS) was precipitated and subjected to trypsin digestion. Phosphorylated peptide enrichment was then carried out using titanium dioxide (TiO₂) beads (GL sciences) and subjected LC-MS/MS using the Q Exactive. Phosphoproteomics datasets were analyzed using PhosphoPath (Raaijmakers et al., J. Proteome Res., 2015, 14, 4332-4341) and Kinase Substrate Enrichment Analysis (KSEA) (Wiredja et al., Bioinformatics, 2017, 33, 3489-3491) software that uses pre-existing phosphoproteomics databases to determine candidate active kinases in samples to predict active kinases. Deep single-run proteome analysis was carried out as previously described (Coscia et al., Nat. Commun., 2016, 7, 12645). Briefly, proteins were extracted from 200 μg of lysates (100 μg of sample, 100 μg of HKS) using methanol/chloroform protein precipitation methods and subjected to trypsin digestion. Peptides were then purified using C-18 Sep-Pak (Waters) and subjected to LC-MS/MS using the Q Exactive.

Various modifications of the described subject matter, in addition to those described herein, will be apparent to those skilled in the art from the foregoing description. Such modifications are also intended to fall within the scope of the appended claims. Each reference (including, but not limited to, journal articles, U.S. and non-U.S. patents, patent application publications, international patent application publications, gene bank accession numbers, and the like) cited in the present application is incorporated herein by reference in its entirety.

1. A multi-analyte column for kinome isolation comprising at least four layers of multiplexer inhibitor beads, wherein:
a first layer comprises a first multiplexed-inhibitor bead having at least one immobilized kinase inhibitor;
a second layer comprises a second multiplexed-inhibitor bead having at least one immobilized kinase inhibitor;
a third layer comprises a third multiplexed-inhibitor bead having at least one immobilized kinase inhibitor;
a fourth layer comprises a fourth multiplexed-inhibitor bead having at least one immobilized kinase inhibitor; and
wherein the immobilized kinase inhibitor in each layer is different from the immobilized kinase inhibitor in the other layers.

2. The multi-analyte column according to claim 1, further comprising:
a fifth layer comprising a fifth multiplexed-inhibitor bead having at least one immobilized kinase inhibitor;
a sixth layer comprising a sixth multiplexed-inhibitor bead having at least one immobilized kinase inhibitor; and
a seventh layer comprising a seventh multiplexed-inhibitor bead having at least one immobilized kinase inhibitor;
wherein the immobilized kinase inhibitor in each layer is different from the immobilized kinase inhibitor in the other layers.

3. The multi-analyte column according to claim 1, wherein the immobilized kinase inhibitor is chosen from bisindolymaleimide-X, GW-572016, SB203580, CTx-0249885, 2,4-diaminopyrimidine, pyrazole, PP58, purvalanol B, VI16832, AX14596, SU6668, dasatinib, lapatinib, afatinib, axitinib, bosutinib, ceritinib, cobimetinib, crizotinib, entrectinib, erlotinib, fostamatinib, gefitinib, ibrutinib, imatinib, lenvatinib, mubritinib, nilotinib, pazopanib, ruxolitinib, sorafenib, sunitinib, SU6656, vandetanib, vemurafenib, A47, CHEMBL592030, CHEMBL1993661, CHEMBL1983268, KCB-A41, AZD3463, FRAX486, PF-06463922, GDL-0941, THZ1, JQ1, MK1775, AZD7762, BDP5290, RKI-1447, SP772077, GSK46317A, A647563, BDP5290, RKI-1447, SP772077, GSK46317A, PF-3758309, PND1186, and PP121.

4. (canceled)

5. The multi-analyte column according to claim 1, wherein the immobilized kinase inhibitors are purvalanol B, VI16832, PP58, and CTx-0249885.

6. The multi-analyte column according to claim 5, wherein:
the kinase inhibitor immobilized on the first multiplexed-inhibitor bead of the first layer is purvalanol B;
the kinase inhibitor immobilized on the second multiplexed-inhibitor bead of the second layer is PP58;
the kinase inhibitor immobilized on the third multiplexed-inhibitor bead of the third layer is VI16832; and
the kinase inhibitor immobilized on the fourth multiplexed-inhibitor bead of the fourth layer is CTx-0249885.

7-9. (canceled)

10. The multi-analyte column according to claim 2, wherein the immobilized kinase inhibitors specific for catalytically active kinases are gefitinib, bisindolymaleimide-X, SB203580, dasatinib, purvalanol B, PP58, and VI16832.

11. The multi-analyte column according to claim 10, wherein:
the kinase inhibitor immobilized on the first multiplexed-inhibitor bead of the first layer is gefitinib;
the kinase inhibitor immobilized on the second multiplexed-inhibitor bead of the second layer is bisindolymaleimide-X;
the kinase inhibitor immobilized on the third multiplexed-inhibitor bead of the third layer is SB203580;
the kinase inhibitor immobilized on the fourth multiplexed-inhibitor bead of the fourth layer is dasatinib;
the kinase inhibitor immobilized on the fifth multiplexed-inhibitor bead of the fifth layer is purvalanol B;
the kinase inhibitor immobilized on the sixth multiplexed-inhibitor bead of the sixth layer is PP58; and
the kinase inhibitor immobilized on the seventh multiplexed-inhibitor bead of the seventh layer is VI16832.

12-13. (canceled)

14. The multi-analyte column according to claim 2, wherein:
the kinase inhibitor immobilized on the first multiplexed-inhibitor bead of the first layer is bisindolymaleimide-X;
the kinase inhibitor immobilized on the second multiplexed-inhibitor bead of the second layer is SB203580;
the kinase inhibitor immobilized on the third multiplexed-inhibitor bead of the third layer is lapatinib;
the kinase inhibitor immobilized on the fourth multiplexed-inhibitor bead of the fourth layer is dasatinib;
the kinase inhibitor immobilized on the fifth multiplexed-inhibitor bead of the fifth layer is purvalanol B;
the kinase inhibitor immobilized on the sixth multiplexed-inhibitor bead of the sixth layer is VI16832; and
the kinase inhibitor immobilized on the seventh multiplexed-inhibitor bead of the seventh layer is PP58.

15-18. (canceled)

19. A method of generating a kinome profile of a cancer cell comprising:
enriching protein kinases from a cancer cell-containing sample obtained from a subject;
detecting the enriched kinases; and
transforming the data obtained from the detected and quantified enriched kinases into the kinome profile.

20. The method according to claim 19, wherein enriching the protein kinases comprises:
loading a lysate from the cancer cell-containing sample on a multi-analyte column, wherein the column comprises: a first layer comprising a first multiplexed-inhibitor bead having at least one immobilized kinase inhibitor, a second layer comprising a second multiplexed-inhibitor bead having at least one immobilized kinase inhibitor, a third layer comprising a third multiplexed-inhibitor bead having at least one immobilized kinase inhibitor, and a fourth layer comprising a fourth multiplexed-inhibitor bead having at least one immobilized kinase inhibitor; wherein the immobilized kinase inhibitor in each layer is different from the immobilized kinase inhibitor in the other three layers;
washing the multi-analyte column to remove any unbound proteins; and
eluting kinases bound to the multi-analyte column with a denaturing agent.

21. The method according to claim 20, wherein the four immobilized kinase inhibitors are purvalanol B, VI16832, PP58, and CTx-0249885.

22. The method according to claim 19, wherein enriching the protein kinases comprises:
loading a lysate from the cancer cell-containing sample on a multi-analyte column, wherein the column comprises: a first layer comprising a first multiplexed-inhibitor bead having at least one immobilized kinase inhibitor, a second layer comprising a second multiplexed-inhibitor bead having at least one immobilized kinase inhibitor, a third layer comprising a third multiplexed-inhibitor bead having at least one immobilized kinase inhibitor, a fourth layer comprising a fourth multiplexed-inhibitor bead having at least one immobilized kinase inhibitor; a fifth layer comprising a fifth multiplexed-inhibitor bead having at least one immobilized kinase inhibitor, a sixth layer comprising a sixth multiplexed-inhibitor bead having at least one immobilized kinase inhibitor, and a seventh layer comprising a seventh multiplexed-inhibitor bead having at least one immobilized kinase inhibitor; wherein the immobilized kinase inhibitor in each layer is different from the immobilized kinase inhibitor in the other six layers;
washing the multi-analyte column to remove any unbound proteins; and
eluting kinases bound to the multi-analyte column with a denaturing agent.

23-48. (canceled)

49. A method of treating cancer in a subject comprising:
generating a kinome profile from a cancer cell-containing sample from a subject;
comparing the kinome profile to a standard cancer kinome profile; and
administering to the subject an effective amount of one or more appropriate kinase inhibitors, chemotherapeutic agents, epigenetic therapy agents, additional biologically active compounds or any combinations thereof.

50. The method according to claim 49, wherein obtaining kinome profile comprises:
enriching protein kinases from a cancer cell-containing sample from a subject;
detecting the enriched kinases; and
transforming the data obtained from the detected and quantified enriched kinases into the kinome profile.

51. The method according to claim 50, wherein enriching the protein kinases comprises:
loading a lysate from the cancer cell-containing sample on a multi-analyte column, wherein the column comprises at least four layers of multiplexer inhibitor beads, wherein each layer comprises a multiplexed-inhibitor bead having at least one immobilized kinase inhibitor, and wherein the immobilized kinase inhibitor in each layer is different from the immobilized kinase inhibitor in the other layers;
washing the multi-analyte column to remove any unbound proteins; and
eluting kinases bound to the multi-analyte column with a denaturing agent.

52-59. (canceled)

60. A method of assessing a cancer therapy regimen comprising:
generating a first kinome profile from a first cancer cell-containing sample obtained from a subject;
treating the subject with one or more kinase inhibitors, chemotherapy agents, or epigenetic therapies;
generating a second kinome profile from a second cancer cell-containing sample obtained from the subject;
comparing the first kinome profile and the second kinome profile; and
modifying the treatment regimen based on the changes in the second kinome profile as compared to the first kinome profile.

61. The method according to claim 60, wherein obtaining kinome profile comprises:
enriching protein kinases from a cancer cell-containing sample from a subject;
detecting the enriched kinases; and
transforming the data obtained from the detected and quantified enriched kinases into the kinome profile.

62. The method according to claim 61, wherein enriching the protein kinases comprises:
loading a lysate from the cancer cell-containing sample on a multi-analyte column, wherein the column comprises at least four layers of multiplexer inhibitor beads, wherein each layer comprises a multiplexed-inhibitor bead having at least one immobilized kinase inhibitor, and wherein the immobilized kinase inhibitor in each layer is different from the immobilized kinase inhibitor in the other layers;
washing the multi-analyte column to remove any unbound proteins; and
eluting kinases bound to the multi-analyte column with a denaturing agent.

63-66. (canceled)

67. A method for predicting the development of resistance to a chemotherapy regimen in a subject, comprising:
generating a kinome profile from a cancer cell-containing sample obtained from the subject; and
comparing the kinome profile to a standard cancer kinome profile;
wherein the presence of the measured level or phosphorylation status of at least one kinase that is comparable to the cancer standard level indicates that the subject is at an increased risk for development of resistance to the chemotherapy regimen.

68. The method according to claim 67, wherein obtaining kinome profile comprises:
enriching protein kinases from a cancer cell-containing sample from a subject;
detecting the enriched kinases; and
transforming the data obtained from the detected and quantified enriched kinases into the kinome profile.

69. The method according to claim 67, wherein enriching the protein kinases comprises:
loading a lysate from the cancer cell-containing sample on a multi-analyte column, wherein the column comprises at least four layers of multiplexer inhibitor beads, wherein each layer comprises a multiplexed-inhibitor bead having at least one immobilized kinase inhibitor, and wherein the immobilized kinase inhibitor in each layer is different from the immobilized kinase inhibitor in the other layers;
washing the multi-analyte column to remove any unbound proteins; and
eluting kinases bound to the multi-analyte column with a denaturing agent.

70-73. (canceled)

74. A method of selecting a kinase activity modulator, the method comprising the steps of:
contacting a cell, a tissue, or an organism with a compound;
contacting a protein extract from the cell, the tissue, or the organism with a multi-analyte column comprising at least four layers of multiplexer inhibitor beads, wherein each layer comprises a multiplexed-inhibitor bead having at least one immobilized kinase inhibitor, and wherein the immobilized kinase inhibitor in each layer is different from the immobilized kinase inhibitor in the other layers;
eluting any kinases bound to the solid supports with a denaturing agent;
measuring levels of a plurality of the kinases detected to generate a kinome profile;
comparing the measured kinome profile to a standard kinome profile obtained from cells that were not contacted with a compound; and
using the kinome profile to select the kinase activity modulator.

75-76. (canceled)

77. A method of treating High Grade Serous Ovarian Carcinoma (HGSOC), in a patient comprising administering to a patient a pharmaceutical composition comprising an effective amount of one or more therapeutic agents that inhibit at least one protein kinase, wherein the protein kinase is BRAF, MEK, ERK, FAK1, P70S6, AURKA, WEE1, CHEK1, CDK7, EphA, ATR, PI3KM/MTOR, CDK4, CDK6, IGF1R, EGFR-HER2, PAN-TK, NUAK, EIF2AK2, STK38, MRCKA, PAK4, PRPKCQ, AAK, CDK1, JAK1, ERBB4, DDR1, FGFR2, AKT2, PTK2B, MAPK1, and MAPK14.

78-89. (canceled)

90. The method according to claim 77, further comprising administering a BET inhibitor to the patient, wherein the BET inhibitor JQ1, OTX015, I-BET-762, RVX-208, I-BET-762, I-BET 151 (GSK1210151A), I-BET 762 (GSK525762), I-BET-726 (GSK1324726A), TEN-010, CPI-203, CPI-0610, RVX-208, ABBV-075, BAY1238097, and LY294002, or any combination thereof.

91-118. (canceled)

119. The method of suppressing carboplatin resistance in a patient having High Grade Serous Ovarian Carcinoma (HGSOC) comprising administering to the patient an effective amount of a pharmaceutical composition comprising an MRCKA inhibitor.

120. (canceled)
